SEQUENCE LISTING (61 sequences)

SEQ ID NO: 1 - DNA, 7918 bp, Artificial Sequence
  pBREW0 RePS-Hyg-URA3-Cas9
gatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtgaaggcaagcccagaaaaatatcgcaagcacctttggtcttacagtgccaacttttggcctgccgacgttaagagtacaaagctgatggcaatgtacgacaagataacagagtctcaaaagaagtgaaacaatttttcttcaccacattttccattgttccttccccccataactataaacgtatttatgtatatatatttgcgtgtaagtgtgtgtactatagggcaccgtaaagtaataatgcttaattagttactactatgaccatataagaggtcatactgtatgaagccacaaagcagatagatcaatcatgtttaacgaaaactgttaatcgaagattatttctttttttttttctctttcctttttacaaagaaaattttttttgcgctttttgccatcaccatcgcaagttctgggacaattgttctctttcgctccagttccaaggaaagaggtttctgttttacttaatagaaagtgtcatcttgtattttatatctcttctttcttgtgtaaaattctttagttttgattttgtatttttaggacagtgagctacgaagtaacatttttacttaataaccgtttgaagcatagagcaggccctggtaccaccacctaatatctggctttttattcaataaaaactcaaaaaaaaaaatccaaaaaaaactaaaaaaccaataaaaataaaatggataagaagtactccatcggtttggatattggtactaactccgtcggttgggctgttattactgacgaatacaaagtcccatctaagaagttcaaggtcttgggtaacactgatagacactccattaagaagaacttaattggtgccttgttattcgattctggtgaaactgccgaagccactcgtttgaagagaactgctagaagacgttacactagaagaaagaaccgtatttgttacttgcaagaaattttctctaatgaaatggctaaagtcgacgattccttcttccacagattggaagaatcttttttggtcgaagaagataaaaagcacgaaagacacccaattttcggtaatattgtcgatgaagtcgcttaccacgaaaagtacccaactatctaccacttgagaaaaaaattggtcgactctaccgataaggccgacttaagattgatttacttggctttggcccacatgattaagttcagaggtcactttttgattgaaggtgatttgaacccagataactccgatgttgataaattattcatccaattagtccaaacttataaccaattgttcgaagaaaacccaatcaacgcttctggtgtcgatgctaaggctattttgtccgctagattgtctaagtctcgtagattggaaaacttgattgctcaattgccaggtgaaaagaagaacggtttgttcggtaacttgattgctttgtccttgggtttgactccaaacttcaagtccaacttcgacttggctgaggatgctaagttacaattatctaaagatacctacgacgatgatttggacaacttattggctcaaattggtgatcaatacgccgatttgttcttagccgctaagaacttgtctgacgctattttgttgtctgacattttgagagttaacactgaaatcaccaaagctccattgtccgcttccatgattaaaagatacgacgaacaccaccaagacttaaccttgttgaaggctttggttagacaacaattgccagaaaagtacaaagaaattttcttcgatcaatctaaaaacggttatgccggttacatcgacggtggtgcctctcaagaagaattctataagttcattaagcctatcttggaaaagatggatggtactgaagaattgttagttaagttgaacagagaagacttgttgcgtaagcaaagaacctttgacaacggttctatccctcaccaaatccacttgggtgaattgcacgctatcttgagaagacaagaggacttctacccattcttaaaggataacagagaaaagatcgaaaaaattttgactttcagaattccatattacgtcggtccattggccagaggtaattctagattcgcttggatgactagaaagtctgaagaaactatcactccatggaatttcgaagaagtcgttgataagggtgcttccgctcaatctttcattgaacgtatgactaacttcgacaaaaatttgcctaacgaaaaggttttgccaaagcactccttgttgtacgaatattttactgtttacaacgaattgactaaggttaagtacgttaccgaaggtatgagaaagccagctttcttgtctggtgaacaaaagaaggccattgttgatttgttgtttaagaccaacagaaaggttactgtcaagcaattgaaagaagattacttcaagaagatcgaatgtttcgattctgtcgagatctccggtgttgaggatagatttaacgcttctttaggtacctaccacgatttattgaagatcatcaaggacaaggatttcttggacaacgaagaaaacgaagatatcttggaagacattgtcttgactttaaccttatttgaagatagagaaatgattgaagaaagattgaagacctacgcccacttgttcgatgacaaggtcatgaagcaattgaaaagaagaagatacaccggttggggtagattatccagaaagttaattaacggtatcagagataagcaatctggtaagaccatcttggatttcttgaagtccgatggtttcgctaacagaaacttcatgcaattgattcatgacgactccttgaccttcaaggaagacattcaaaaagctcaagtttccggtcaaggtgactctttgcatgaacacatcgctaacttggccggttccccagctattaagaagggtattttgcaaaccgttaaggtcgtcgacgaattagttaaggtcatgggtagacacaagccagaaaacattgttattgaaatggctagagaaaatcaaaccactcaaaaaggtcaaaaaaactccagagaaagaatgaagagaattgaagaaggtattaaagaattgggttcccaaattttaaaggaacacccagttgaaaatactcaattacaaaacgagaagttgtatttgtactatttacaaaacggtagagatatgtacgtcgaccaagaattagacatcaaccgtttatctgactacgacgtcgatcacatcgtcccacaatctttcttgaaggacgattctatcgacaacaaggttttaactcgttctgacaagaatagaggtaaatccgataacgttccatccgaagaagtcgtcaaaaagatgaaaaactactggagacaattgttgaacgctaagttgatcacccaaagaaaatttgataacttaactaaggctgaacgtggtggtttgtccgaattggacaaggccggtttcattaagagacaattagttgaaacccgtcaaattactaagcacgttgctcaaattttggattcccgtatgaacactaagtacgatgaaaacgacaagttgattagagaggtcaaggttattaccttgaagtccaagttggtttccgacttcagaaaggattttcaattttacaaagttcgtgaaatcaacaactatcaccacgctcacgatgcttacttaaacgccgttgtcggtaccgctttgattaaaaagtatccaaagttggaatccgaattcgtctacggtgactacaaggtctacgacgtcagaaaaatgattgctaagtctgaacaagaaattggtaaggctactgctaagtacttcttctattccaacatcatgaacttttttaaaaccgaaatcaccttggctaatggtgaaatcagaaaaagacctttgatcgaaactaacggtgaaaccggtgaaattgtttgggataagggtagagacttcgctaccgttagaaaggttttgtccatgccacaagtcaacattgtcaagaagaccgaagttcaaaccggtggtttctctaaggaatctatcttgccaaagcgtaattctgacaaattgattgccagaaagaaggattgggatccaaaaaaatacggtggtttcgattctccaactgttgcttactccgtcttggtcgtcgctaaagtcgaaaagggtaagtctaagaagttgaaatccgtcaaagaattgttgggtatcactattatggaaagatcttctttcgaaaagaacccaatcgatttcttggaagccaagggttacaaggaagttaaaaaggacttgatcattaaattgccaaaatactctttgttcgaattggagaatggtagaaaaagaatgttggcttccgctggtgaattgcaaaagggtaacgaattggctttgccatccaagtacgttaactttttgtacttagcctctcactacgaaaagttgaaaggttccccagaagataacgaacaaaaacaattgttcgtcgaacaacataaacattatttggatgagattattgaacaaatttctgagttttctaagagagtcatcttggccgacgctaacttggataaggtcttgtccgcctacaacaagcacagagacaagccaatcagagaacaagccgaaaacatcattcacttgttcactttaactaacttgggtgccccagctgctttcaaatacttcgacactaccattgacagaaagagatacacttctactaaggaagtcttggatgctactttgatccaccaatccatcactggtttgtacgaaactagaattgatttgtctcaattgggtggtgatagcagggctgaccccaagaagaagaggaaggtgtagtatataactgtctagaaataaacacccgtcgagcctgtccgatttcaaagtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgaaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccggg

SEQ ID NO: 2 - DNA, 6783 bp, Unknown
  Removal by Prototrophic Selection (RePS) vector RePS-A sequence
gatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtgaaggcaagcccagaaaaatatcgcaagcacctttggtcttacagtgccaacttttggcctgccgacgttaagagtacaaagctgatggcaatgtacgacaagataacagagtctcaaaagaagtgaaacaatttttcttcaccacattttccattgttccttccccccataactataaacgtatttatgtatatatatttgcgtgtaagtgtgtgtactatagggcaccgtaaagtaataatgcttaattagttactactatgaccatataagaggtcatactgtatgaagccacaaagcagatagatcaatcatgtttaacgaaaactgttaatcgaagattatttctttttttttttctctttcctttttacaaagaaaattttttttgcgctttttgccatcaccatcgcaagttctgggacaattgttctctttcgctccagttccaaggaaagaggtttctgttttacttaatagaaagtgtcatcttgtattttatatctcttctttcttgtgtaaaattctttagttttgattttgtatttttaggacagtgagctacgaagtaacatttttacttaataaccgtttgaagcatagagcaggccctggtaccaccacctaatatctggctttttattcaataaaaactcaaaaaaaaaaatccaaaaaaaactaaaaaaccaataaaaataaaatggataagaagtactccatcggtttggatattggtactaactccgtcggttgggctgttattactgacgaatacaaagtcccatctaagaagttcaaggtcttgggtaacactgatagacactccattaagaagaacttaattggtgccttgttattcgattctggtgaaactgccgaagccactcgtttgaagagaactgctagaagacgttacactagaagaaagaaccgtatttgttacttgcaagaaattttctctaatgaaatggctaaagtcgacgattccttcttccacagattggaagaatcttttttggtcgaagaagataaaaagcacgaaagacacccaattttcggtaatattgtcgatgaagtcgcttaccacgaaaagtacccaactatctaccacttgagaaaaaaattggtcgactctaccgataaggccgacttaagattgatttacttggctttggcccacatgattaagttcagaggtcactttttgattgaaggtgatttgaacccagataactccgatgttgataaattattcatccaattagtccaaacttataaccaattgttcgaagaaaacccaatcaacgcttctggtgtcgatgctaaggctattttgtccgctagattgtctaagtctcgtagattggaaaacttgattgctcaattgccaggtgaaaagaagaacggtttgttcggtaacttgattgctttgtccttgggtttgactccaaacttcaagtccaacttcgacttggctgaggatgctaagttacaattatctaaagatacctacgacgatgatttggacaacttattggctcaaattggtgatcaatacgccgatttgttcttagccgctaagaacttgtctgacgctattttgttgtctgacattttgagagttaacactgaaatcaccaaagctccattgtccgcttccatgattaaaagatacgacgaacaccaccaagacttaaccttgttgaaggctttggttagacaacaattgccagaaaagtacaaagaaattttcttcgatcaatctaaaaacggttatgccggttacatcgacggtggtgcctctcaagaagaattctataagttcattaagcctatcttggaaaagatggatggtactgaagaattgttagttaagttgaacagagaagacttgttgcgtaagcaaagaacctttgacaacggttctatccctcaccaaatccacttgggtgaattgcacgctatcttgagaagacaagaggacttctacccattcttaaaggataacagagaaaagatcgaaaaaattttgactttcagaattccatattacgtcggtccattggccagaggtaattctagattcgcttggatgactagaaagtctgaagaaactatcactccatggaatttcgaagaagtcgttgataagggtgcttccgctcaatctttcattgaacgtatgactaacttcgacaaaaatttgcctaacgaaaaggttttgccaaagcactccttgttgtacgaatattttactgtttacaacgaattgactaaggttaagtacgttaccgaaggtatgagaaagccagctttcttgtctggtgaacaaaagaaggccattgttgatttgttgtttaagaccaacagaaaggttactgtcaagcaattgaaagaagattacttcaagaagatcgaatgtttcgattctgtcgagatctccggtgttgaggatagatttaacgcttctttaggtacctaccacgatttattgaagatcatcaaggacaaggatttcttggacaacgaagaaaacgaagatatcttggaagacattgtcttgactttaaccttatttgaagatagagaaatgattgaagaaagattgaagacctacgcccacttgttcgatgacaaggtcatgaagcaattgaaaagaagaagatacaccggttggggtagattatccagaaagttaattaacggtatcagagataagcaatctggtaagaccatcttggatttcttgaagtccgatggtttcgctaacagaaacttcatgcaattgattcatgacgactccttgaccttcaaggaagacattcaaaaagctcaagtttccggtcaaggtgactctttgcatgaacacatcgctaacttggccggttccccagctattaagaagggtattttgcaaaccgttaaggtcgtcgacgaattagttaaggtcatgggtagacacaagccagaaaacattgttattgaaatggctagagaaaatcaaaccactcaaaaaggtcaaaaaaactccagagaaagaatgaagagaattgaagaaggtattaaagaattgggttcccaaattttaaaggaacacccagttgaaaatactcaattacaaaacgagaagttgtatttgtactatttacaaaacggtagagatatgtacgtcgaccaagaattagacatcaaccgtttatctgactacgacgtcgatcacatcgtcccacaatctttcttgaaggacgattctatcgacaacaaggttttaactcgttctgacaagaatagaggtaaatccgataacgttccatccgaagaagtcgtcaaaaagatgaaaaactactggagacaattgttgaacgctaagttgatcacccaaagaaaatttgataacttaactaaggctgaacgtggtggtttgtccgaattggacaaggccggtttcattaagagacaattagttgaaacccgtcaaattactaagcacgttgctcaaattttggattcccgtatgaacactaagtacgatgaaaacgacaagttgattagagaggtcaaggttattaccttgaagtccaagttggtttccgacttcagaaaggattttcaattttacaaagttcgtgaaatcaacaactatcaccacgctcacgatgcttacttaaacgccgttgtcggtaccgctttgattaaaaagtatccaaagttggaatccgaattcgtctacggtgactacaaggtctacgacgtcagaaaaatgattgctaagtctgaacaagaaattggtaaggctactgctaagtacttcttctattccaacatcatgaacttttttaaaaccgaaatcaccttggctaatggtgaaatcagaaaaagacctttgatcgaaactaacggtgaaaccggtgaaattgtttgggataagggtagagacttcgctaccgttagaaaggttttgtccatgccacaagtcaacattgtcaagaagaccgaagttcaaaccggtggtttctctaaggaatctatcttgccaaagcgtaattctgacaaattgattgccagaaagaaggattgggatccaaaaaaatacggtggtttcgattctccaactgttgcttactccgtcttggtcgtcgctaaagtcgaaaagggtaagtctaagaagttgaaatccgtcaaagaattgttgggtatcactattatggaaagatcttctttcgaaaagaacccaatcgatttcttggaagccaagggttacaaggaagttaaaaaggacttgatcattaaattgccaaaatactctttgttcgaattggagaatggtagaaaaagaatgttggcttccgctggtgaattgcaaaagggtaacgaattggctttgccatccaagtacgttaactttttgtacttagcctctcactacgaaaagttgaaaggttccccagaagataacgaacaaaaacaattgttcgtcgaacaacataaacattatttggatgagattattgaacaaatttctgagttttctaagagagtcatcttggccgacgctaacttggataaggtcttgtccgcctacaacaagcacagagacaagccaatcagagaacaagccgaaaacatcattcacttgttcactttaactaacttgggtgccccagctgctttcaaatacttcgacactaccattgacagaaagagatacacttctactaaggaagtcttggatgctactttgatccaccaatccatcactggtttgtacgaaactagaattgatttgtctcaattgggtggtgatagcagggctgaccccaagaagaagaggaaggtgtagtatataactgtctagaaataaacacccgtcgagcctgtccgatttcaaagtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcag

SEQ ID NO: 3 - DNA, 182 bp, Unknown
  promoter pURA3
gatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatc

SEQ ID NO: 4 - DNA, 568 bp, Unknown
  URA3 flanking sequence 1
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtg

SEQ ID NO: 5 - DNA, 299 bp, Artificial Sequence
  direct repeat loop-out sequence
aagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggt

SEQ ID NO: 6 - DNA, 700 bp, Unknown
  promoter pPAB1
aaggcaagcccagaaaaatatcgcaagcacctttggtcttacagtgccaacttttggcctgccgacgttaagagtacaaagctgatggcaatgtacgacaagataacagagtctcaaaagaagtgaaacaatttttcttcaccacattttccattgttccttccccccataactataaacgtatttatgtatatatatttgcgtgtaagtgtgtgtactatagggcaccgtaaagtaataatgcttaattagttactactatgaccatataagaggtcatactgtatgaagccacaaagcagatagatcaatcatgtttaacgaaaactgttaatcgaagattatttctttttttttttctctttcctttttacaaagaaaattttttttgcgctttttgccatcaccatcgcaagttctgggacaattgttctctttcgctccagttccaaggaaagaggtttctgttttacttaatagaaagtgtcatcttgtattttatatctcttctttcttgtgtaaaattctttagttttgattttgtatttttaggacagtgagctacgaagtaacatttttacttaataaccgtttgaagcatagagcaggccctggtaccaccacctaatatctggctttttattcaataaaaactcaaaaaaaaaaatccaaaaaaaactaaaaaaccaataaaaataaa

SEQ ID NO: 7 - DNA, 4140 bp, Unknown
  Cas9 coding sequence
atggataagaagtactccatcggtttggatattggtactaactccgtcggttgggctgttattactgacgaatacaaagtcccatctaagaagttcaaggtcttgggtaacactgatagacactccattaagaagaacttaattggtgccttgttattcgattctggtgaaactgccgaagccactcgtttgaagagaactgctagaagacgttacactagaagaaagaaccgtatttgttacttgcaagaaattttctctaatgaaatggctaaagtcgacgattccttcttccacagattggaagaatcttttttggtcgaagaagataaaaagcacgaaagacacccaattttcggtaatattgtcgatgaagtcgcttaccacgaaaagtacccaactatctaccacttgagaaaaaaattggtcgactctaccgataaggccgacttaagattgatttacttggctttggcccacatgattaagttcagaggtcactttttgattgaaggtgatttgaacccagataactccgatgttgataaattattcatccaattagtccaaacttataaccaattgttcgaagaaaacccaatcaacgcttctggtgtcgatgctaaggctattttgtccgctagattgtctaagtctcgtagattggaaaacttgattgctcaattgccaggtgaaaagaagaacggtttgttcggtaacttgattgctttgtccttgggtttgactccaaacttcaagtccaacttcgacttggctgaggatgctaagttacaattatctaaagatacctacgacgatgatttggacaacttattggctcaaattggtgatcaatacgccgatttgttcttagccgctaagaacttgtctgacgctattttgttgtctgacattttgagagttaacactgaaatcaccaaagctccattgtccgcttccatgattaaaagatacgacgaacaccaccaagacttaaccttgttgaaggctttggttagacaacaattgccagaaaagtacaaagaaattttcttcgatcaatctaaaaacggttatgccggttacatcgacggtggtgcctctcaagaagaattctataagttcattaagcctatcttggaaaagatggatggtactgaagaattgttagttaagttgaacagagaagacttgttgcgtaagcaaagaacctttgacaacggttctatccctcaccaaatccacttgggtgaattgcacgctatcttgagaagacaagaggacttctacccattcttaaaggataacagagaaaagatcgaaaaaattttgactttcagaattccatattacgtcggtccattggccagaggtaattctagattcgcttggatgactagaaagtctgaagaaactatcactccatggaatttcgaagaagtcgttgataagggtgcttccgctcaatctttcattgaacgtatgactaacttcgacaaaaatttgcctaacgaaaaggttttgccaaagcactccttgttgtacgaatattttactgtttacaacgaattgactaaggttaagtacgttaccgaaggtatgagaaagccagctttcttgtctggtgaacaaaagaaggccattgttgatttgttgtttaagaccaacagaaaggttactgtcaagcaattgaaagaagattacttcaagaagatcgaatgtttcgattctgtcgagatctccggtgttgaggatagatttaacgcttctttaggtacctaccacgatttattgaagatcatcaaggacaaggatttcttggacaacgaagaaaacgaagatatcttggaagacattgtcttgactttaaccttatttgaagatagagaaatgattgaagaaagattgaagacctacgcccacttgttcgatgacaaggtcatgaagcaattgaaaagaagaagatacaccggttggggtagattatccagaaagttaattaacggtatcagagataagcaatctggtaagaccatcttggatttcttgaagtccgatggtttcgctaacagaaacttcatgcaattgattcatgacgactccttgaccttcaaggaagacattcaaaaagctcaagtttccggtcaaggtgactctttgcatgaacacatcgctaacttggccggttccccagctattaagaagggtattttgcaaaccgttaaggtcgtcgacgaattagttaaggtcatgggtagacacaagccagaaaacattgttattgaaatggctagagaaaatcaaaccactcaaaaaggtcaaaaaaactccagagaaagaatgaagagaattgaagaaggtattaaagaattgggttcccaaattttaaaggaacacccagttgaaaatactcaattacaaaacgagaagttgtatttgtactatttacaaaacggtagagatatgtacgtcgaccaagaattagacatcaaccgtttatctgactacgacgtcgatcacatcgtcccacaatctttcttgaaggacgattctatcgacaacaaggttttaactcgttctgacaagaatagaggtaaatccgataacgttccatccgaagaagtcgtcaaaaagatgaaaaactactggagacaattgttgaacgctaagttgatcacccaaagaaaatttgataacttaactaaggctgaacgtggtggtttgtccgaattggacaaggccggtttcattaagagacaattagttgaaacccgtcaaattactaagcacgttgctcaaattttggattcccgtatgaacactaagtacgatgaaaacgacaagttgattagagaggtcaaggttattaccttgaagtccaagttggtttccgacttcagaaaggattttcaattttacaaagttcgtgaaatcaacaactatcaccacgctcacgatgcttacttaaacgccgttgtcggtaccgctttgattaaaaagtatccaaagttggaatccgaattcgtctacggtgactacaaggtctacgacgtcagaaaaatgattgctaagtctgaacaagaaattggtaaggctactgctaagtacttcttctattccaacatcatgaacttttttaaaaccgaaatcaccttggctaatggtgaaatcagaaaaagacctttgatcgaaactaacggtgaaaccggtgaaattgtttgggataagggtagagacttcgctaccgttagaaaggttttgtccatgccacaagtcaacattgtcaagaagaccgaagttcaaaccggtggtttctctaaggaatctatcttgccaaagcgtaattctgacaaattgattgccagaaagaaggattgggatccaaaaaaatacggtggtttcgattctccaactgttgcttactccgtcttggtcgtcgctaaagtcgaaaagggtaagtctaagaagttgaaatccgtcaaagaattgttgggtatcactattatggaaagatcttctttcgaaaagaacccaatcgatttcttggaagccaagggttacaaggaagttaaaaaggacttgatcattaaattgccaaaatactctttgttcgaattggagaatggtagaaaaagaatgttggcttccgctggtgaattgcaaaagggtaacgaattggctttgccatccaagtacgttaactttttgtacttagcctctcactacgaaaagttgaaaggttccccagaagataacgaacaaaaacaattgttcgtcgaacaacataaacattatttggatgagattattgaacaaatttctgagttttctaagagagtcatcttggccgacgctaacttggataaggtcttgtccgcctacaacaagcacagagacaagccaatcagagaacaagccgaaaacatcattcacttgttcactttaactaacttgggtgccccagctgctttcaaatacttcgacactaccattgacagaaagagatacacttctactaaggaagtcttggatgctactttgatccaccaatccatcactggtttgtacgaaactagaattgatttgtctcaattgggtggtgatagcagggctgaccccaagaagaagaggaaggtgtag

SEQ ID NO: 8 - DNA, 33 bp, Artificial Sequence
  engineered tag NLS
agcagggctgaccccaagaagaagaggaaggtg

SEQ ID NO: 9 - DNA, 49 bp, Unknown
  terminator Tsynth11
tatataactgtctagaaataaacacccgtcgagcctgtccgatttcaaa

SEQ ID NO: 10 - DNA, 369 bp, Unknown
  promoter Ag-pTEF
gtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 11 - DNA, 1029 bp, Unknown
  Hph gene
atgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataa

SEQ ID NO: 12 - DNA, 1445 bp, Artificial Sequence
  Removal by Prototrophic Selection (RePS) vector RePS-B sequence
gctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgaaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccggg

SEQ ID NO: 13 - DNA, 264 bp, Unknown
  terminator KanMX_term
tcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatga

SEQ ID NO: 14 - DNA, 536 bp, Unknown
  URA3 flanking sequence 2
aagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 15 - DNA, 299 bp, Artificial Sequence; identical to SEQ ID NO: 5
  direct repeat loop-out

SEQ ID NO: 16 - DNA, 81 bp, Unknown
  terminator tURA3
aaaactgtattataagtaaatgcatgtatactaaactcacaaattagagcttcaatttaattatatcagttattacccggg

SEQ ID NO: 17 - DNA, 8942 bp, Artificial Sequence
  pGUIDE-7.1-with-MCH5
aacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcactgggtggaatcccttctgcagcacctggattaccctgttatccctagtacagcccacgttagtccgtcaaattcaggggagatcaccgttgagtcctcatctccctcaagcaggccggccgtagactgccatcgagtctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcccaaagatggtgagtcacgtgttttagagctagaaatagcaagttaaaataaggctagtccgttatcaacttgaaaaagtggcaccgagtcggtggtgctttttttgttttttatgtctcattaccagggaccggagttctggttaattaacaggctcctggtccagagtaccgatctatttgctgatcggtacggtgggctgatcggcgataacctgagctggacggcgacgtaaacgcgcgcgttaggaacgtacccagtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgaactgaaaattttttgtttgcttctctctctctctctttcattatgtgagatttaaaaaccagaaactacatcatcgatgtgaagagcttcactgagtagggcccgggctgtaaacggtcgatgttccgcggagaggtttgttatccccggcgttaggaactacggtggcagatgtagtgtttccacagggcgatcgcctactggtagcaggtagaactatgcggtgtgaaataccgccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacacggtgagtgagtgtgtgcgtgtggggcgcgccagatgggaacaggatcttgaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgactccagtctttctagaagatggcaaacagctattatgggtattatgggtatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcactttactgatacgtgtccagatcaaccgctttcacgacctctaccagacacatgtgatcacggcgctcgtcgcggtctttgctcagtttggtgtggtaggtaatgtgatgataacgcgggatatgcactgccgcggagcccgccaacggacgattcatttggctgcatttggtaaccagtttttcggtcacaccttcaatatcgtacgcctggttgaactcaacgcggatgccattgttaacggtgtcaggcagaatatacagaatgcttggcgggcattggaatgcaacgttcttacgcagaatgtgaccgtctttcttaaagttctcaccagtcagcgtgacacgattgtagatagaaccgcgttcgtaggtaaccatagcacgcgtcttgtacacgccgtcgccttcgaagctgatggtacgctcttgggtataaccttccggcatggcgctcttaaagaaatccttgatgtggctcgggtacttggcgaaacactgaacaccgtagctcagggtgctcaccagggttgcccacgggaccggcaggtcgcccgtagtgcagatgtatttcgctttaatggtacccgtggtcgcgtcaccggtaccctcgcctttaatgataaatttcataccttcgacgtcgccttccagttcggtgatatacgggatctctttctcaaacagttttgcaccttccgtcaatgccgtcattttgtaattaaaacttagattagattgctatgctttctttctaatgagcaagaagtaaaaaaagttgtaatagaacaagaaaaatgaaactgaaacttgagaaattgatgaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtgtcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgggggatcacttgtgggggattgggtgtgatgtaaggattcgcggtcctcgaaaattaaaagtccaacgcgcctgttgcttcctatgtgatatgtattatatgtaatatgcataaatatatctactgcattgtattttgaacgtacaaagtatgcattgtttatacgctattatcagccaaagttgggtggtcgctttctgttgtatgactattgatgtctaggctgtcaataatttcgttttgagcctccatgtctctgaagaactccctgttggcaaggaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 18 - DNA, 50 bp, Unknown
  cloned region tag_9
ggtgagtgagtgtgtgcgtgtggggcgcgccagatgggaacaggatcttg

SEQ ID NO: 19 - DNA, 804 bp, Unknown
  Kl_URA3 coding sequence
atgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaa

SEQ ID NO: 20 - DNA, 40 bp, Artificial Sequence
  cloned region stuffer 4
acagcccacgttagtccgtcaaattcaggggagatcaccg

SEQ ID NO: 21 - DNA, 50 bp, Artificial Sequence
  cloned region tag 10
actccagtctttctagaagatggcaaacagctattatgggtattatgggt

SEQ ID NO: 22 - DNA, 50 bp, Artificial Sequence
  cloned region tag 3
attaccagggaccggagttctggttaattaacaggctcctggtccagagt

SEQ ID NO: 23 - DNA, 711 bp, Unknown
  DasherGFP CDS gene
ttactgatacgtgtccagatcaaccgctttcacgacctctaccagacacatgtgatcacggcgctcgtcgcggtctttgctcagtttggtgtggtaggtaatgtgatgataacgcgggatatgcactgccgcggagcccgccaacggacgattcatttggctgcatttggtaaccagtttttcggtcacaccttcaatatcgtacgcctggttgaactcaacgcggatgccattgttaacggtgtcaggcagaatatacagaatgcttggcgggcattggaatgcaacgttcttacgcagaatgtgaccgtctttcttaaagttctcaccagtcagcgtgacacgattgtagatagaaccgcgttcgtaggtaaccatagcacgcgtcttgtacacgccgtcgccttcgaagctgatggtacgctcttgggtataaccttccggcatggcgctcttaaagaaatccttgatgtggctcgggtacttggcgaaacactgaacaccgtagctcagggtgctcaccagggttgcccacgggaccggcaggtcgcccgtagtgcagatgtatttcgctttaatggtacccgtggtcgcgtcaccggtaccctcgcctttaatgataaatttcataccttcgacgtcgccttccagttcggtgatatacgggatctctttctcaaacagttttgcaccttccgtcaatgccgtcat

SEQ ID NO: 24 - DNA, 50 bp, Artificial Sequence
  cloned region tag 7
tgtgaagagcttcactgagtagggcccgggctgtaaacggtcgatgttcc

SEQ ID NO: 25 - DNA, 269 bp, Unknown
  pSNR52 sequence
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatc

SEQ ID NO: 26 - DNA, 94 bp, Unknown
  sgRNA structural sequence
gttttagagctagaaatagcaagttaaaataaggctagtccgttatcaacttgaaaaagtggcaccgagtcggtggtgctttttttgtttttta

SEQ ID NO: 27 - DNA, 1343 bp, Unknown
  origin of replication 2u ori sequence
aacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatc

SEQ ID NO: 28 - DNA, 50 bp, Artificial Sequence
  cloned region tag 4
ataacctgagctggacggcgacgtaaacgcgcgcgttaggaacgtaccca

SEQ ID NO: 29 - DNA, 48 bp, Unknown
  protein binding site FRT
gaagttcctatactttctagagaataggaacttcggaataggaacttc

SEQ ID NO: 30 - DNA, 40 bp, Artificial Sequence
  cloned region stuffer 5
accgatctatttgctgatcggtacggtgggctgatcggcg

SEQ ID NO: 31 - DNA, 595 bp, Unknown
  origin of replication MBI ORI sequence
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcc

SEQ ID NO: 32 - DNA, 20 bp, Artificial Sequence
  MCH5 spacer
ccaaagatggtgagtcacgt

SEQ ID NO: 33 - DNA, 197 bp, Unknown
  terminator T_URA3
attatacaggaaacttaatagaacaaatcacatatttaatctaatagccacctgcattggcacggtgcaacactacttcaacttcatcttacaaaaagatcacgtgatctgttgtattgaactgaaaattttttgtttgcttctctctctctctctttcattatgtgagatttaaaaaccagaaactacatcatcga

SEQ ID NO: 34 - DNA, 283 bp, Unknown
  promoter P_URA3
gtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcacc

SEQ ID NO: 35 - DNA, 795 bp, Unknown
  nptII gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 36 - DNA, 50 bp, Artificial Sequence
  cloned region tag 2
ttgagtcctcatctccctcaagcaggccggccgtagactgccatcgagtc

SEQ ID NO: 37 - DNA, 50 bp, Artificial Sequence
  cloned region tag 8
gcagatgtagtgtttccacagggcgatcgcctactggtagcaggtagaac

SEQ ID NO: 38 - DNA, 1000 bp, Unknown
  promoter pADH2
aatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 39 - DNA, 225 bp, Unknown
  terminator ScENO2
atttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcact

SEQ ID NO: 40 - DNA, 40 bp, Artificial Sequence
  stuffer 8
gcggagaggtttgttatccccggcgttaggaactacggtg

SEQ ID NO: 41 - DNA, 50 bp, Artificial Sequence
  cloned region tag 1
actgggtggaatcccttctgcagcacctggattaccctgttatccctagt

SEQ ID NO: 42 - DNA, 700 bp, Unknown
  promoter ScTEF1
tttgtaattaaaacttagattagattgctatgctttctttctaatgagcaagaagtaaaaaaagttgtaatagaacaagaaaaatgaaactgaaacttgagaaattgatgaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtgtcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgggggatcacttgtgggggattgggtgtgatgtaaggattcgcggtcctcgaaaattaaaagtccaacgcgcctgttgcttcctatgtgatatgtattatatgtaatatgcataaatatatctactgcattgtattttgaacgtacaaagtatgcattgtttatacgctattatcagccaaagttgggtggtcgctttctgttgtatgactattgatgtctaggctgtcaataatttcgttttgagcctccatgtctctgaagaactccctgttggcaagg

SEQ ID NO: 43 - DNA, 29 bp, Artificial Sequence
  Tag1-2micron primer
aatgctatcatttcctttgatattggatc

SEQ ID NO: 44 - DNA, 6502 bp, Artificial Sequence
  RePS-Trp1-Kan
gtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactgccaacatttttgtttcttttggacaaatgttgtttgcatttatgatccgttatattttgatctaatgtagagttgcacgtagttcttactggcaaagaaatcgatgcataccaaaaaagaataaaggtgatatttgatctttaccgtttagttccaacgtaaaattgtgcctttggacttaaaatggcgtggcagaactaactctttatttttccaaatcagaaaaattaatatgttttgccgcgttaaaacctacatcaaaaaaggcggatcaagatgtatgaaagaaagtgcgtagaataacgaacattcatagctgttctggaggctttacaaaaggtaatctttgttaggtgcgattttatccgaaaagcaattactctctatgttagtcatacaaactgacttctgtgagagaattattttcatatcaacgtaagatcacatggttcctttatcaagtactactatcattccattatatgacctatttacttcttgaatcttagagctcataattcaagcaagttgcggagctaagaatttcacatgttgttgaacttaacaatcttcattatacccaatcgctgcgtgctggaattatgttaaaagttacatcctttttttcatttttccctacgctcagggcactgtactgcccgtgcctgcgatgagatacatcaatttaaaaaaaaaaccagcatgctataatgctggagcaaaaatttcaatcagaaatagaaaagacctcaacagtaattaacccaaaggggtatcaaataatcgatgtgctttttcactctacgaatgatctgtgagaaactgatttgggccgaatcgcgtaaaaagtttgattcgtggcggctaatgtctgaggggctccaacaggctcgtagagcctcgtttcttgagggcacaaaatgtccaggtaatattcccaagaaagaaccgcagagtgctttgataaatcggttacaggtcttaacgtaggttttgtctcgctaattgctattgagtaagttcgatccgtttggcgtcttttggggtgtaacgccaaacttattacttttcctatttgaggttggtattgattgttgtcaaagaatgaaaatatacacaaacgccacaatatacgtaccaggttcacgaaaactgatcgtatggttcataccctgacttggcaaacctaatgtgaccgtcgctgattagcggatcacgaaaagtgatctcgatacaattagaggatccacgaaaatgatgtgaatgaatacatgaaagattcatgagatctgacaacatggtagacgtgtgtgtctcatggaaattgatgcagttgaagacatgtgcgtcacgaaaaaagaaatcaatcctacacagggcttaagggcaaatgtattcatgtgtgtcacgaaaagtgatgtaactaaatacacgattaccatggaaattaacgtaccttttttgtgcgtgtattgaaatattatgacatattacagaaagggttcgcaagtcctgtttctatgcctttctcttagtaattcacgaaataaacctatggtttacgaaatgatccacgaaaatcatgttattatttacatcaacatatcgcgaaaattcatgtcatgtccacattaacatcattgcagagcaacaattcattttcatagagaaatttgctactatcacccactagtactaccattggtacctactactttgaattgtactaccgctgggcgttattaggtgtgaaaccacgaaaagttcaccataacttcgaataaagtcgcggaaaaaagtaaacagctattgctactcaaatgaggtttgcagaagcttgttgaagcatgatgaagcgttctaaacgcactattcatcattaaatatttaaagctcataaaattgtattcaattcctattctaaatggcttttatttctattacaactattagctctaaatccatatcctcataagcagcaatcaattctatctatactttaaaatgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcacaggaaatggtgttttatctaaatttctcactggacgtagtgaccttgtaactcctgctgtaaaaagtatggcttggatgcttggtctgtggttaggtgacggtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaactttacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcataaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaaaatgtgtatattagtttaaaaagttgtatgtaataaaagtaaaatttaatattttggatgaaaaaaaccatttttagactttttcttaactagaatgctggagtagaaatacgccatctcaagatacaaaaagcgttaccggcactgatttgtttcaaccagtatatagattattattgggtcttgatcaactttcctcagacatatcagtaacagttatcaagctaaatatttacgcgaaagaaaaacaaatattttaattgtgatacttgtgaattttattttattaaggatacaaagttaagagaaaacaaaagtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgattgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 45 - DNA, 475 bp, Unknown
  TRP1 flanking sequence 1
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaact

SEQ ID NO: 46 - DNA, 5440 bp, Artificial Sequence
  Removal by Prototrophic Selection (RePS) vector RePS-A sequence
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactgccaacatttttgtttcttttggacaaatgttgtttgcatttatgatccgttatattttgatctaatgtagagttgcacgtagttcttactggcaaagaaatcgatgcataccaaaaaagaataaaggtgatatttgatctttaccgtttagttccaacgtaaaattgtgcctttggacttaaaatggcgtggcagaactaactctttatttttccaaatcagaaaaattaatatgttttgccgcgttaaaacctacatcaaaaaaggcggatcaagatgtatgaaagaaagtgcgtagaataacgaacattcatagctgttctggaggctttacaaaaggtaatctttgttaggtgcgattttatccgaaaagcaattactctctatgttagtcatacaaactgacttctgtgagagaattattttcatatcaacgtaagatcacatggttcctttatcaagtactactatcattccattatatgacctatttacttcttgaatcttagagctcataattcaagcaagttgcggagctaagaatttcacatgttgttgaacttaacaatcttcattatacccaatcgctgcgtgctggaattatgttaaaagttacatcctttttttcatttttccctacgctcagggcactgtactgcccgtgcctgcgatgagatacatcaatttaaaaaaaaaaccagcatgctataatgctggagcaaaaatttcaatcagaaatagaaaagacctcaacagtaattaacccaaaggggtatcaaataatcgatgtgctttttcactctacgaatgatctgtgagaaactgatttgggccgaatcgcgtaaaaagtttgattcgtggcggctaatgtctgaggggctccaacaggctcgtagagcctcgtttcttgagggcacaaaatgtccaggtaatattcccaagaaagaaccgcagagtgctttgataaatcggttacaggtcttaacgtaggttttgtctcgctaattgctattgagtaagttcgatccgtttggcgtcttttggggtgtaacgccaaacttattacttttcctatttgaggttggtattgattgttgtcaaagaatgaaaatatacacaaacgccacaatatacgtaccaggttcacgaaaactgatcgtatggttcataccctgacttggcaaacctaatgtgaccgtcgctgattagcggatcacgaaaagtgatctcgatacaattagaggatccacgaaaatgatgtgaatgaatacatgaaagattcatgagatctgacaacatggtagacgtgtgtgtctcatggaaattgatgcagttgaagacatgtgcgtcacgaaaaaagaaatcaatcctacacagggcttaagggcaaatgtattcatgtgtgtcacgaaaagtgatgtaactaaatacacgattaccatggaaattaacgtaccttttttgtgcgtgtattgaaatattatgacatattacagaaagggttcgcaagtcctgtttctatgcctttctcttagtaattcacgaaataaacctatggtttacgaaatgatccacgaaaatcatgttattatttacatcaacatatcgcgaaaattcatgtcatgtccacattaacatcattgcagagcaacaattcattttcatagagaaatttgctactatcacccactagtactaccattggtacctactactttgaattgtactaccgctgggcgttattaggtgtgaaaccacgaaaagttcaccataacttcgaataaagtcgcggaaaaaagtaaacagctattgctactcaaatgaggtttgcagaagcttgttgaagcatgatgaagcgttctaaacgcactattcatcattaaatatttaaagctcataaaattgtattcaattcctattctaaatggcttttatttctattacaactattagctctaaatccatatcctcataagcagcaatcaattctatctatactttaaaatgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcacaggaaatggtgttttatctaaatttctcactggacgtagtgaccttgtaactcctgctgtaaaaagtatggcttggatgcttggtctgtggttaggtgacggtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaactttacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcataaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaaaatgtgtatattagtttaaaaagttgtatgtaataaaagtaaaatttaatattttggatgaaaaaaaccatttttagactttttcttaactagaatgctggagtagaaatacgccatctcaagatacaaaaagcgttaccggcactgatttgtttcaaccagtatatagattattattgggtcttgatcaactttcctcagacatatcagtaacagttatcaagctaaatatttacgcgaaagaaaaacaaatattttaattgtgatacttgtgaattttattttattaaggatacaaagttaagagaaaacaaaagtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggc

SEQ ID NO: 47 - DNA, 300 bp, Artificial Sequence
  direct repeat loop out
ttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaact

SEQ ID NO: 48 - DNA, 2002 bp, Unknown
  HO promoter
gccaacatttttgtttcttttggacaaatgttgtttgcatttatgatccgttatattttgatctaatgtagagttgcacgtagttcttactggcaaagaaatcgatgcataccaaaaaagaataaaggtgatatttgatctttaccgtttagttccaacgtaaaattgtgcctttggacttaaaatggcgtggcagaactaactctttatttttccaaatcagaaaaattaatatgttttgccgcgttaaaacctacatcaaaaaaggcggatcaagatgtatgaaagaaagtgcgtagaataacgaacattcatagctgttctggaggctttacaaaaggtaatctttgttaggtgcgattttatccgaaaagcaattactctctatgttagtcatacaaactgacttctgtgagagaattattttcatatcaacgtaagatcacatggttcctttatcaagtactactatcattccattatatgacctatttacttcttgaatcttagagctcataattcaagcaagttgcggagctaagaatttcacatgttgttgaacttaacaatcttcattatacccaatcgctgcgtgctggaattatgttaaaagttacatcctttttttcatttttccctacgctcagggcactgtactgcccgtgcctgcgatgagatacatcaatttaaaaaaaaaaccagcatgctataatgctggagcaaaaatttcaatcagaaatagaaaagacctcaacagtaattaacccaaaggggtatcaaataatcgatgtgctttttcactctacgaatgatctgtgagaaactgatttgggccgaatcgcgtaaaaagtttgattcgtggcggctaatgtctgaggggctccaacaggctcgtagagcctcgtttcttgagggcacaaaatgtccaggtaatattcccaagaaagaaccgcagagtgctttgataaatcggttacaggtcttaacgtaggttttgtctcgctaattgctattgagtaagttcgatccgtttggcgtcttttggggtgtaacgccaaacttattacttttcctatttgaggttggtattgattgttgtcaaagaatgaaaatatacacaaacgccacaatatacgtaccaggttcacgaaaactgatcgtatggttcataccctgacttggcaaacctaatgtgaccgtcgctgattagcggatcacgaaaagtgatctcgatacaattagaggatccacgaaaatgatgtgaatgaatacatgaaagattcatgagatctgacaacatggtagacgtgtgtgtctcatggaaattgatgcagttgaagacatgtgcgtcacgaaaaaagaaatcaatcctacacagggcttaagggcaaatgtattcatgtgtgtcacgaaaagtgatgtaactaaatacacgattaccatggaaattaacgtaccttttttgtgcgtgtattgaaatattatgacatattacagaaagggttcgcaagtcctgtttctatgcctttctcttagtaattcacgaaataaacctatggtttacgaaatgatccacgaaaatcatgttattatttacatcaacatatcgcgaaaattcatgtcatgtccacattaacatcattgcagagcaacaattcattttcatagagaaatttgctactatcacccactagtactaccattggtacctactactttgaattgtactaccgctgggcgttattaggtgtgaaaccacgaaaagttcaccataacttcgaataaagtcgcggaaaaaagtaaacagctattgctactcaaatgaggtttgcagaagcttgttgaagcatgatgaagcgttctaaacgcactattcatcattaaatatttaaagctcataaaattgtattcaattcctattctaaatggcttttatttctattacaactattagctctaaatccatatcctcataagcagcaatcaattctatctatacttt

SEQ ID NO: 49 - DNA, 4082 bp, Artificial Sequence
  HO endonuclease transcription unit
gccaacatttttgtttcttttggacaaatgttgtttgcatttatgatccgttatattttgatctaatgtagagttgcacgtagttcttactggcaaagaaatcgatgcataccaaaaaagaataaaggtgatatttgatctttaccgtttagttccaacgtaaaattgtgcctttggacttaaaatggcgtggcagaactaactctttatttttccaaatcagaaaaattaatatgttttgccgcgttaaaacctacatcaaaaaaggcggatcaagatgtatgaaagaaagtgcgtagaataacgaacattcatagctgttctggaggctttacaaaaggtaatctttgttaggtgcgattttatccgaaaagcaattactctctatgttagtcatacaaactgacttctgtgagagaattattttcatatcaacgtaagatcacatggttcctttatcaagtactactatcattccattatatgacctatttacttcttgaatcttagagctcataattcaagcaagttgcggagctaagaatttcacatgttgttgaacttaacaatcttcattatacccaatcgctgcgtgctggaattatgttaaaagttacatcctttttttcatttttccctacgctcagggcactgtactgcccgtgcctgcgatgagatacatcaatttaaaaaaaaaaccagcatgctataatgctggagcaaaaatttcaatcagaaatagaaaagacctcaacagtaattaacccaaaggggtatcaaataatcgatgtgctttttcactctacgaatgatctgtgagaaactgatttgggccgaatcgcgtaaaaagtttgattcgtggcggctaatgtctgaggggctccaacaggctcgtagagcctcgtttcttgagggcacaaaatgtccaggtaatattcccaagaaagaaccgcagagtgctttgataaatcggttacaggtcttaacgtaggttttgtctcgctaattgctattgagtaagttcgatccgtttggcgtcttttggggtgtaacgccaaacttattacttttcctatttgaggttggtattgattgttgtcaaagaatgaaaatatacacaaacgccacaatatacgtaccaggttcacgaaaactgatcgtatggttcataccctgacttggcaaacctaatgtgaccgtcgctgattagcggatcacgaaaagtgatctcgatacaattagaggatccacgaaaatgatgtgaatgaatacatgaaagattcatgagatctgacaacatggtagacgtgtgtgtctcatggaaattgatgcagttgaagacatgtgcgtcacgaaaaaagaaatcaatcctacacagggcttaagggcaaatgtattcatgtgtgtcacgaaaagtgatgtaactaaatacacgattaccatggaaattaacgtaccttttttgtgcgtgtattgaaatattatgacatattacagaaagggttcgcaagtcctgtttctatgcctttctcttagtaattcacgaaataaacctatggtttacgaaatgatccacgaaaatcatgttattatttacatcaacatatcgcgaaaattcatgtcatgtccacattaacatcattgcagagcaacaattcattttcatagagaaatttgctactatcacccactagtactaccattggtacctactactttgaattgtactaccgctgggcgttattaggtgtgaaaccacgaaaagttcaccataacttcgaataaagtcgcggaaaaaagtaaacagctattgctactcaaatgaggtttgcagaagcttgttgaagcatgatgaagcgttctaaacgcactattcatcattaaatatttaaagctcataaaattgtattcaattcctattctaaatggcttttatttctattacaactattagctctaaatccatatcctcataagcagcaatcaattctatctatactttaaaatgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcacaggaaatggtgttttatctaaatttctcactggacgtagtgaccttgtaactcctgctgtaaaaagtatggcttggatgcttggtctgtggttaggtgacggtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaactttacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcataaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaaaatgtgtatattagtttaaaaagttgtatgtaataaaagtaaaatttaatattttggatgaaaaaaaccatttttagactttttcttaactagaatgctggagtagaaatacgccatctcaagatacaaaaagcgttaccggcactgatttgtttcaaccagtatatagattattattgggtcttgatcaactttcctcagacatatcagtaacagttatcaagctaaatatttacgcgaaagaaaaacaaatattttaattgtgatacttgtgaattttattttattaaggatacaaagttaagagaaaacaaaa

SEQ ID NO: 50 - DNA, 400 bp, Unknown
  regulatory sequence URS1
tgttaaaagttacatcctttttttcatttttccctacgctcagggcactgtactgcccgtgcctgcgatgagatacatcaatttaaaaaaaaaaccagcatgctataatgctggagcaaaaatttcaatcagaaatagaaaagacctcaacagtaattaacccaaaggggtatcaaataatcgatgtgctttttcactctacgaatgatctgtgagaaactgatttgggccgaatcgcgtaaaaagtttgattcgtggcggctaatgtctgaggggctccaacaggctcgtagagcctcgtttcttgagggcacaaaatgtccaggtaatattcccaagaaagaaccgcagagtgctttgataaatcggttacaggtcttaacgtaggttttgtctcgct

SEQ ID NO: 51 - DNA, 1761 bp, Unknown
  HO endonuclease gene
atgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcacaggaaatggtgttttatctaaatttctcactggacgtagtgaccttgtaactcctgctgtaaaaagtatggcttggatgcttggtctgtggttaggtgacggtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaactttacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcataaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaa

SEQ ID NO: 52 - DNA, 50 bp, Artificial Sequence
  HO-del-L sequence
aatgtgtatattagtttaaaaagttgtatgtaataaaagtaaaatttaat

SEQ ID NO: 53 - DNA, 316 bp, Unknown
  HO/YDL228C intergenic terminator
aatgtgtatattagtttaaaaagttgtatgtaataaaagtaaaatttaatattttggatgaaaaaaaccatttttagactttttcttaactagaatgctggagtagaaatacgccatctcaagatacaaaaagcgttaccggcactgatttgtttcaaccagtatatagattattattgggtcttgatcaactttcctcagacatatcagtaacagttatcaagctaaatatttacgcgaaagaaaaacaaatattttaattgtgatacttgtgaattttattttattaaggatacaaagttaagagaaaacaaaa

SEQ ID NO: 54 - DNA, 53 bp, Unknown
  Autonomously Replicating Sequence ARS404
taaaagtaaaatttaatattttggatgaaaaaaaccatttttagactttttct

SEQ ID NO: 55 - DNA, 369 bp, Unknown; identical to SEQ ID NO: 10
  Ag-pTEF promoter

SEQ ID NO: 56 - DNA, 810 bp, Unknown
  KanMX6_G418_resistance gene
atgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 57 - DNA, 1269 bp, Artificial Sequence
  Removal by Prototrophic Selection (RePS) vector RePS-B sequence
aacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgattgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 58 - DNA, 209 bp, Artificial Sequence
  marker overlap sequence
aacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggc

SEQ ID NO: 59 - DNA, 264 bp, Unknown; identical to SEQ ID NO: 13
  KanMX terminator

SEQ ID NO: 60 - DNA, 500 bp, Unknown
  TRP1 flanking sequence 2
ttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 61 - DNA, 300 bp, Artificial Sequence; identical to SEQ ID NO: 47
  direct repeat loop out

(19) United States
(12) Patent Application Publication
(10) Pub. No.: US 2023/0340539 A1
(43) Pub. Date: Oct. 26, 2023

(54) METHODS OF TRANSIENT PROTEIN AND GENE EXPRESSION IN CELLS

(71) Applicant: Zymergen Inc., Emeryville, CA (US)

(72) Inventors: Colin Scott MAXWELL, Oakland, CA (US); Solomon Henry STONEBLOOM, Alameda, CA (US); Shawn SZYJKA, Martinez, CA (US)

(21) Appl. No.: 17/927,336

(22) PCT Filed: May 25, 2021
(86) PCT No.: PCT/US2021/034087

§ 371 Date: Nov. 22, 2022

Related U.S. Application Data
(60) Provisional application No. 63/030,007, filed on May 26, 2020.

(51) Int. Cl. C12N 15/90; C12N 15/11; C12N 9/22; C12N 1/16; C12N 15/79

(52) U.S. Cl. CPC C12N 15/905; C12N 15/11; C12N 9/22; C12N 1/16; C12N 15/79; C12N 2310/20; C12N 2800/80

(57) ABSTRACT

The present disclosure provides methods for producing gene-edited cells free of gene-editing system molecules through the manipulation of prototrophy. Exemplary system molecules include those required for CRISPR editing techniques, such as plasmids and genes encoding Cas nucleases. The methods may employ constructs that temporarily disrupt prototrophy, the removal of which restores prototrophy. Also disclosed are gene-edited cells and populations of gene-edited cells comprising these constructs. The present methods and compositions may be used to achieve desired gene editing of a host cell in the absence of extraneous genetic material remaining from the genetic engineering technique itself.

### CROSS-REFERENCE TO RELATED APPLICATIONS

This application claims the benefit of priority to U.S. Provisional Application No. 63/030,007, filed on May 26, 2020, the content of which is herein incorporated by reference in its entirety.

### INCORPORATION OF THE SEQUENCE LISTING

The contents of the text file submitted electronically herewith are incorporated herein by reference in their entirety: A computer readable format copy of the Sequence Listing (filename: ZYMR_055_01WO_SeqList_ST25.txt, date recorded May 25, 2021, file size ˜93 KB).

### FIELD OF THE DISCLOSURE

The present disclosure provides methods for producing gene-edited cells free of gene-editing system molecules through the manipulation of prototrophy. Exemplary system molecules include those required for CRISPR editing techniques, such as plasmids and genes encoding such molecules. The methods may employ constructs that temporarily disrupt prototrophy, the removal of which restores prototrophy. Also disclosed are gene-edited cells and populations of gene-edited cells comprising these constructs. The present methods and compositions may be used to achieve desired gene editing of a host cell in the absence of extraneous genetic material remaining from the genetic engineering technique itself.

### BACKGROUND

CRISPR gene editing is a commonly used genetic engineering technique by which the genomes of living organisms may be modified. It is based on a simplified version of the bacterial CRISPR-Cas9 antiviral defense system. In many organisms, genome editing using CRISPR nucleases such as Cas9 or Cas12a may involve the introduction of DNA encoding two components: DNA expressing the Cas nuclease, and DNA expressing the guide RNA (gRNA). However, use of CRISPR gene editing suffers from three notable difficulties.

First, in applications requiring a strain without exogenous DNA remaining in the cell (for example, during a fermentation), DNA expressing different guide RNAs must be introduced and sequentially removed from the organism. This often requires multiple rounds of genetic engineering to introduce and then remove the guide RNAs.

Second, plasmids containing selectable/counterselectable metabolic genes are an attractive method to introduce and then remove plasmids expressing gRNAs. However, this requires the use of auxotrophic strains which depend on the presence of the plasmid to provide the required metabolic gene or require specially supplemented growth media. Auxotrophic strains are undesirable for use in fermentation as their metabolism may differ substantially from prototrophic strains. Thus it is desirable to restore the prototrophy of a strain before use in a fermentation, which traditionally requires an additional transformation to re-introduce a construct expressing the wild-type metabolic gene.

Third, expressing the Cas nuclease from DNA integrated into the genome of an organism can have advantages over expression from plasmids due to lower toxicity and less cell-to-cell variability. However, in many cases, the DNA encoding the Cas nuclease must then be removed from the organism before it can be used in downstream processes (e.g. in fermentations), which necessitates further manipulation of the cell genome to achieve the desired result.

Each of these challenges add time, expense, and difficulty to the process of genetic engineering through CRISPR.

Within yeast, alternative genome editing methods make use of mating to combine desired gene edits of interest from different strains. However, these methods are complicated by the desire to obtain haploid yeast cells from a process that requires mating competent yeast that produce diploid cells.

There is an ongoing and unmet need for improved methods to streamline genetic engineering and the removal of extraneous genetic material left over from the engineering process.

### BRIEF SUMMARY

In one aspect, the present disclosure provides a method for producing a population of gene-edited cells free of gene-editing system molecules, comprising: (a) introducing an integrating nucleic acid construct into a population of cells that comprise a target gene of interest and that are prototrophic for a nutrient, wherein the integrating nucleic acid construct integrates into a gene that is required for prototrophy for the nutrient; and wherein the integrating nucleic acid construct comprises: a first nucleotide sequence encoding a gene-editing protein; a second nucleotide sequence encoding a dominant selectable marker; and a pair of repeat nucleotide sequences flanking the first nucleotide sequence and the second nucleotide sequence; (b) selecting for expression of the dominant selectable marker to produce a population of cells that are auxotrophic for the nutrient; (c) introducing a non-integrating nucleic acid construct into the population of cells produced in step (b); wherein the non-integrating nucleic acid construct comprises: a third nucleotide sequence encoding a gene-editing nucleic acid that introduces an edit into the gene of interest; and a fourth nucleotide sequence encoding a protein that complements the auxotrophy for the nutrient, wherein the fourth nucleotide sequence cannot recombine with the cellular genome; (d) simultaneously selecting for expression of the dominant selectable marker and for prototrophy for the nutrient to produce a population of cells that comprise the edited gene of interest; (e) removing the non-integrating nucleic acid nucleic acid construct from the population of cells produced in step (d) by growing the cells on media that selects against expression of the protein that complements the auxotrophy for the nutrient to produce a population of cells that comprise the edited gene of interest and are free of the non-integrating nucleic acid construct; and (f) removing the integrating nucleic acid construct from the population of cells produced in step (e) by growing the cells on media that selects for prototrophy for the nutrient to produce a population of cells that comprise the edited gene of interest and that are free of the integrating nucleic acid construct.

In some embodiments, the cells are fungal cells or bacterial cells.

In some embodiments, the fungal cells are Fusarium spp., Kluyveromyces spp., Penicillium spp., Pichia spp., Saccharomyces spp., Schizosaccharomyces spp. or Yarrowia spp.

In some embodiments, the fungal cells are Kluyveromyces lactis, Kluyveromyces marxianus, Pichia pastoris, Saccharomyces cerevisiae, Schizosaccharomyces pombe or Yarrowia lipolytica.

In some embodiments, the bacterial cells are Agrobacterium spp., Arthrobacterspecies spp., Bacillus spp., Clostridium spp., Corynebacterium spp., Cupriavidus spp., Escherichia spp., Erwinia spp., Geobacillus spp., Lactobacillus spp., Pantoea spp., Propionibacterium spp., Pseudomonas spp., Sphingomonas spp., Streptococcus spp., Streptomyces spp., Xanthomonas spp., or Zymomonas spp.

In some embodiments, the bacterial cells are Bacillus clausii, Bacillus lichenifonnis, Bacillus subtilis, Clostridium acetobutylicum, Corynebacterium glutamicum, Cupriavidus necator, Escherichia coli, Geobacillus thermoglucosidasius, Propionibacterium freudenreichii, Sphingomonas elodea, or Xanthomonas campestris.

In some embodiments, the gene-editing protein is an endonuclease.

In some embodiments, the endonuclease is an RNA-guided endonuclease.

In some embodiments, the RNA-guided endonuclease is a CRISPR Class 2 endonuclease.

In some embodiments, the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.

In some embodiments, the CRISPR Class 2 endonuclease is cas9 or cas12a.

In some embodiments, the gene-editing nucleic acid is a guide RNA (gRNA).

In some embodiments, the guide RNA is a single guide RNA (sgRNA).

In some embodiments, the RNA-guided endonuclease is a CRISPR Class 1 endonuclease.

In some embodiments, the CRISPR Class 1 endonuclease is Cas3 or Cas10.

In some embodiments, the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

In some embodiments, the gene that is required for prototrophy for the nutrient is URA3, LYS2, LYS5, CAN1, amdS, FCY1, FCA1, GAP1, HSV_TK or TRP1.

In some embodiments, the protein that complements the auxotrophy for the nutrient is Kluyveromyces lactis URA3 (K1URA3).

In some embodiments, the media that selects against expression of the protein that complements the auxotrophy for the nutrient comprises 5-FOA, alpha-aminoadipate, canavanine, fluoroacetamide, 5-fluorocytosine, D-histidine, antifolate media, or 5-fluoroanthranilic acid.

In some embodiments, the nutrient is uracil, lysine, arginine, acetamide, cytosine, L-citrulline, FUdR or tryptophan.

In some embodiments, the non-integrating nucleic acid construct is a plasmid.

In one aspect, the present disclosure provides a method for producing a population of gene-edited Saccharomyces cerevisiae cells free of Cas9 and sgRNA, comprising: (a) introducing an integrating nucleic acid construct into a population of S. cerevisiae cells that comprise a target gene of interest and that are prototrophic for uracil, wherein the integrating nucleic acid construct integrates into the URA3 gene; and wherein the integrating nucleic acid construct comprises: a first nucleotide sequence encoding Cas9; a second nucleotide sequence encoding HygR; and a pair of repeat nucleotide sequences flanking the first nucleotide sequence and the second nucleotide sequence; (b) selecting for expression of HygR to produce a population of cells that are auxotrophic for uracil; (c) introducing a non-integrating nucleic acid construct into the population of cells produced in step (b); wherein the non-integrating nucleic acid construct comprises: a third nucleotide sequence encoding an sgRNA that introduces an edit into the gene of interest; and a fourth nucleotide sequence encoding Kluyveromyces lactis URA3 (K1URA3) protein; (d) simultaneously selecting for expression of HygR and for prototrophy for uracil to produce a population of cells that comprise the edited gene of interest; (e) removing the non-integrating nucleic acid nucleic acid construct from the population of cells produced in step (d) by growing the cells on media that selects against expression of K1URA3 protein to produce a population of cells that comprise the edited gene of interest and are free of the non-integrating nucleic acid construct; and (f) removing the integrating nucleic acid construct from the population of cells produced in step (e) by growing the cells on media that selects for prototrophy for uracil to produce a population of cells that comprise the edited gene of interest and that are free of the integrating nucleic acid construct.

In one aspect, the present disclosure provides a population of cells comprising a nucleic acid construct integrated into a gene that is required for prototrophy for a nutrient, wherein the integrated nucleic acid construct comprises: a first nucleotide sequence encoding a gene-editing protein; a second nucleotide sequence encoding a dominant selectable marker; and a pair of repeat nucleotide sequences flanking the first nucleotide sequence and the second nucleotide sequence.

In some embodiments, the non-integrating nucleic acid construct comprises: a third nucleotide sequence encoding a gene-editing nucleic acid that introduces an edit into a gene of interest; and a fourth nucleotide sequence encoding a protein that complements the auxotrophy for the nutrient, wherein the fourth nucleotide sequence cannot recombine with the cellular genome.

In one aspect, the present disclosure provides a population of cells comprising an edited gene of interest and a nucleic acid construct integrated into a gene that is required for prototrophy for a nutrient, wherein the integrated nucleic acid construct comprises: a first nucleotide sequence encoding a gene-editing protein; a second nucleotide sequence encoding a dominant selectable marker; and a pair of repeat nucleotide sequences flanking the first nucleotide sequence and the second nucleotide sequence.

In some embodiments, the cells are fungal cells or bacterial cells.

In some embodiments, the fungal cells are Fusarium spp., Kluyveromyces spp., Penicillium spp., Pichia spp., Saccharomyces spp., Schizosaccharomyces spp. or Yarrowia spp.

In some embodiments, the fungal cells are Kluyveromyces lactis, Kluyveromyces marxianus, Pichia pastoris, Saccharomyces cerevisiae, Schizosaccharomyces pombe or Yarrowia lipolytica.

In some embodiments, the bacterial cells are Agrobacterium spp., Arthrobacterspecies spp., Bacillus spp., Clostridium spp., Corynebacterium spp., Cupriavidus spp., Escherichia spp., Erwinia spp., Geobacillus spp., Lactobacillus spp., Pantoea spp., Propionibacterium spp., Pseudomonas spp., Sphingomonas spp., Streptococcus spp., Streptomyces spp., Xanthomonas spp., or Zymomonas spp.

In some embodiments, the bacterial cells are Bacillus clausii, Bacillus lichenifonnis, Bacillus subtilis, Clostridium acetobutylicum, Corynebacterium glutamicum, Cupriavidus necator, Escherichia coli, Geobacillus thermoglucosidasius, Propionibacterium freudenreichii, Sphingomonas elodea, or Xanthomonas campestris.

In some embodiments, the gene-editing protein is an endonuclease.

In some embodiments, the endonuclease is an RNA-guided endonuclease.

In some embodiments, the RNA-guided endonuclease is a CRISPR Class 2 endonuclease.

In some embodiments, the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.

In some embodiments, the CRISPR Class 2 endonuclease is cas9 or cas12a.

In some embodiments, the gene-editing nucleic acid is a guide RNA (gRNA).

In some embodiments, the guide RNA is a single guide RNA (sgRNA).

In some embodiments, the RNA-guided endonuclease is a CRISPR Class 1 endonuclease.

In some embodiments, the CRISPR Class 1 endonuclease is Cas3 or Cas10.

In some embodiments, the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

In some embodiments, the gene that is required for prototrophy for the nutrient is URA3, LYS2, LYS5, CAN1, amdS, FCY1, FCA1, GAP1, HSV_TK or TRP1.

In some embodiments, the protein that complements the auxotrophy for the nutrient is Kluyveromyces lactis URA3 (K1URA3).

In some embodiments, the nutrient is uracil, lysine, arginine, acetamide, cytosine, L-citrulline, FUdR or tryptophan.

In some embodiments, the non-integrating nucleic acid construct is a plasmid.

In one aspect, the present disclosure provides a method for producing a population of multiply gene-edited cells free of gene-editing system molecules, comprising: (a) introducing a first integrating nucleic acid construct into a first population of cells that comprise a first edited gene of interest and that are prototrophic for a nutrient, wherein the first integrating nucleic acid construct integrates into a gene that is required for prototrophy for the nutrient; and wherein the first integrating nucleic acid construct comprises: a first nucleotide sequence encoding a protein that enables mating; a second nucleotide sequence encoding a first dominant selectable marker; and a pair of repeat nucleotide sequences flanking the first nucleotide sequence and the second nucleotide sequence; (b) introducing a second integrating nucleic acid construct into a second population of cells that comprise a second edited gene of interest and that are prototrophic for a nutrient, wherein the second integrating nucleic acid construct integrates into a gene that is required for prototrophy for the nutrient; and wherein the second integrating nucleic acid construct comprises: a third nucleotide sequence encoding a protein that enables mating; a fourth nucleotide sequence encoding a second dominant selectable marker; and a pair of repeat nucleotide sequences flanking the third nucleotide sequence and the fourth nucleotide sequence; (c) selecting for expression of the first dominant selectable marker within the first population of cells and selecting for expression of the second dominant selectable marker within the second population of cells to produce first and second populations of cells that are auxotrophic for the nutrient and mating-competent; (d) sporulating the first and second population of cells of step (c) to produce first and second populations of meiotic progeny; (e) allowing the first and second populations of meiotic progeny to mate with each other, thereby producing a mated population of cells; (f) simultaneously selecting for expression of the first and second dominant selectable markers within the mated population of cells to produce cells comprising genetic information from both the first and second populations of cells; (g) sporulating the mated population of cells of step (f) to allow recombination of the first and second edited genes of interest into a single genome; and (h) removing the integrating nucleic acid construct from the population of cells produced in step (g) by growing the cells on media that selects for prototrophy for the nutrient to produce a population of cells that comprise the edited genes of interest and that are free of the integrating nucleic acid constructs.

In some embodiments, the cells are fungal cells.

In some embodiments, the fungal cells are Fusarium spp., Kluyveromyces spp., Penicillium spp., Pichia spp., Saccharomyces spp., Schizosaccharomyces spp. or Yarrowia spp.

In some embodiments, the fungal cells are Kluyveromyces lactis, Kluyveromyces marxianus, Pichia pastoris, Saccharomyces cerevisiae, Schizosaccharomyces pombe or Yarrowia lipolytica.

In some embodiments, the protein that enables mating is one that enables mating-type switching.

In some embodiments, the protein is the HO endonuclease.

In some embodiments, the first or second dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

In some embodiments, the gene that is required for prototrophy for the nutrient is URA3, LYS2, LYS5, CAN1, amdS, FCY1, FCA1, GAP1, HSV_TK, or TRP1.

In some embodiments, the nutrient is uracil, lysine, arginine, acetamide, cytosine, L-citrulline, FUdR, or tryptophan.

In one aspect, the present disclosure provides a method for producing a population of multiply gene-edited yeast cells free of HO nuclease and antibiotic resistance markers, comprising: (a) introducing a first integrating nucleic acid construct into a first population of haploid yeast cells that comprise a first edited gene of interest and that are prototrophic for tryptophan, wherein the first integrating nucleic acid construct integrates into the TRP1 gene; and wherein the first integrating nucleic acid construct comprises: a first nucleotide sequence encoding HO nuclease; a second nucleotide sequence encoding a kanamycin or hygromycin antibiotic resistance gene; and a pair of repeat nucleotide sequences flanking the first nucleotide sequence and the second nucleotide sequence; (b) introducing a second integrating nucleic acid construct into a second population of haploid yeast cells that comprise a second edited gene of interest and that are prototrophic for tryptophan, wherein the second integrating nucleic acid construct integrates into the TRP1 gene; and wherein the second integrating nucleic acid construct comprises: a third nucleotide sequence encoding HO nuclease; a fourth nucleotide sequence encoding the other of a kanamycin or hygromycin antibiotic resistance gene not encoded by the second nucleotide sequence; and a pair of repeat nucleotide sequences flanking the third nucleotide sequence and the fourth nucleotide sequence; (c) selecting for expression of the antibiotic resistance gene encoded by the second nucleotide sequence within the first population of yeast cells and selecting for expression of the antibiotic resistance gene encoded by the fourth nucleotide sequence within the second population of yeast cells to produce first and second populations of cells that are auxotrophic for tryptophan and mating-competent; (d) sporulating the first and second population of yeast cells of step (c) to produce first and second populations of meiotic progeny; (e) allowing the first and second populations of auxotrophic, mating-competent yeast cells to mate with each other, thereby producing a mated population of cells; (f) simultaneously selecting for expression of both antibiotic resistance genes within the mated population of yeast cells to produce yeast cells comprising genetic information from both the first and second populations of yeast cells; (g) sporulating the mated population of yeast cells of step (f) to allow recombination of the first and second edited genes of interest into a single genome; and (h) removing the integrating nucleic acid construct from the population of yeast cells produced in step (e) by growing the yeast cells on media that selects for tryptophan prototrophy to produce a population of yeast cells that comprise the edited genes of interest and that are free of the integrating nucleic acid constructs.

In one aspect, the present disclosure provides a population of cells comprising a nucleic acid construct integrated into a gene that is required for prototrophy for a nutrient, wherein the integrated nucleic acid construct comprises: a first nucleotide sequence encoding a protein that enables mating; a second nucleotide sequence encoding a dominant selectable marker; and a pair of repeat nucleotide sequences flanking the first nucleotide sequence and the second nucleotide sequence.

In one aspect, the present disclosure provides a population of cells comprising multiple edited genes of interest and two nucleic acid constructs integrated into a gene that is required for prototrophy for a nutrient, wherein the first integrated nucleic acid construct comprises: a first nucleotide sequence encoding a protein that enables mating; a second nucleotide sequence encoding a dominant selectable marker; and a pair of repeat nucleotide sequences flanking the first nucleotide sequence and the second nucleotide sequence; and wherein the second integrated nucleic acid construct comprises: a third nucleotide sequence encoding a protein that enables mating; a fourth nucleotide sequence encoding a second dominant selectable marker; and a pair of repeat nucleotide sequences flanking the third nucleotide sequence and the fourth nucleotide sequence.

In some embodiments, the cells are fungal cells.

In some embodiments, the fungal cells are Fusarium spp., Kluyveromyces spp., Penicillium spp., Pichia spp., Saccharomyces spp., Schizosaccharomyces spp. or Yarrowia spp.

In some embodiments, the fungal cells are Kluyveromyces lactis, Kluyveromyces marxianus, Pichia pastoris, Saccharomyces cerevisiae, Schizosaccharomyces pombe or Yarrowia lipolytica.

In some embodiments, the protein that enables mating is one that enables mating-type switching.

In some embodiments, the protein is the HO endonuclease.

In some embodiments, the first or second dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

In some embodiments, the gene that is required for prototrophy for the nutrient is URA3, LYS2, LYS5, CAN1, amdS, FCY1, FCA1, GAP1, HSV_TK, or TRP1.

In some embodiments, the nutrient is uracil, lysine, arginine, acetamide, cytosine, L-citrulline, FUdR, or tryptophan.

In one aspect, the present disclosure provides a Removal by Prototrophic Selection (RePS) polynucleotide for genetic engineering via integration into a gene that is required for prototrophy for a nutrient, the polynucleotide comprising (a) a first nucleotide sequence encoding a gene-editing protein or a protein that enables mating; (b) a second nucleotide sequence encoding a dominant selectable marker; and (c) a pair of repeat nucleotide sequences flanking the first nucleotide sequence and the second nucleotide sequence, wherein the repeats of (c) allow for recombination to restore the gene that is required for prototrophy for the nutrient while removing the first and second nucleotide sequences.

In some embodiments, the gene-editing protein is an endonuclease.

In some embodiments, the endonuclease is an RNA-guided endonuclease.

In some embodiments, the RNA-guided endonuclease is a CRISPR Class 2 endonuclease.

In some embodiments, the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.

In some embodiments, the CRISPR Class 2 endonuclease is cas9 or cas12a.

In some embodiments, the RNA-guided endonuclease is a CRISPR Class 1 endonuclease.

In some embodiments, the CRISPR Class 1 endonuclease is Cas3 or Cas10.

In some embodiments, the protein that enables mating is one that enables mating-type switching.

In some embodiments, the protein is the HO endonuclease.

In some embodiments, the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

In some embodiments, the gene that is required for prototrophy for the nutrient is URA3, LYS2, LYS5, CAN1, amdS, FCY1, FCA1, GAP1, HSV_TK, or TRP1.

In some embodiments, the nutrient is uracil, lysine, arginine, acetamide, cytosine, L-citrulline, FUdR, or tryptophan.

### BRIEF DESCRIPTION OF THE SEVERAL VIEWS OF THE DRAWINGS

FIG. 1A-FIG. 1F show an overview of an exemplary method according to the present disclosure. FIG. 1A shows a haploid yeast S. cerevisiae with a gene of interest (GOI) and a functioning URA3 gene, making it a uracil prototroph. FIG. 1B shows that the URA3 gene is disrupted by a Removal by Prototrophic Selection (RePS) vector (1), which comprises nucleotide sequences encoding Cas9 nuclease and hygromycin resistance flanked by URA3 repeat sequences that when recombined restore a wild-type allele of URA3. In FIG. 1C, genome editing is accomplished by introducing a plasmid (2) expressing the desired sgRNA using selection for the K1URA3 gene. FIG. 1D shows that the plasmid is removed by 5-FOA selection. In FIG. 1E, the Cas9 nuclease is removed by selection for uracil. FIG. 1F shows that the final strain is a uracil prototroph with an edited genome, and sensitive to hygromycin. In FIG. 1B-FIG. 1D, in order to maintain the Cas9 nuclease in the genome, cells are grown in media containing hygromycin, which selects against loop-out of Cas9.

FIG. 2 shows the results of spot plating for three yeast cell cultures with integrated Cas9-HygR cassettes—(1), (2), (3)—compared to wild type yeast cells (WT) and URA3 knockout cells (−ura3) on different media types. For the media, “SD”=synthetic dextrose, “−ura”=media lacking uracil, “+Hyg”=media containing hygromycin, and “+5FOA”=media containing 5-FOA.

FIG. 3 shows the results of spot-plating for yeast cells with integrated Cas9-HygR cassettes transformed with different combinations of circularized backbone, linear backbone, sgRNA fragments, and repair fragments. Plates had SD+Hyg-ura media. C=Circular backbone; M=MCH5 sgRNA; NT=Non-targeting sgRNA.

FIG. 4A-FIG. 4C provide an overview of an exemplary method of using Removal by Prototrophic Selection (RePS) vectors for genome engineering using yeast mating. FIG. 4A shows step 1: transforming haploid starting strains with RePS vectors. FIG. 4B shows step 2: sporulating, random mating, and selecting for heterozygotes with double antibiotic resistance. FIG. 4C shows step 3: sporulating, selecting for prototrophs formed during meiosis, and screening for the genotype of interest.

FIG. 5 depicts an exemplary embodiment of an automated system for carrying out the methods of the present disclosure. The present disclosure teaches use of automated robotic systems with various modules capable of cloning, transforming, culturing, screening and/or sequencing host organisms.

FIG. 6 depicts the DNA assembly and transformation steps of one of the embodiments of the present disclosure. The flow chart depicts the steps for building DNA fragments, cloning said DNA fragments into vectors, transforming said vectors into host strains, and looping out selection sequences through counter selection.

### DETAILED DESCRIPTION

The present disclosure provides methods of editing the genome of a host strain without leaving residual gene editing nucleic acid sequences behind. In some embodiments, the methods employ the manipulation of prototrophy and/or auxotrophy within the host strain. In some embodiments, the methods comprise the use of both integrating and non-integrating nucleic acid constructs. In some embodiments, the methods comprise the strategic use of selectable markers, selection, counterselection, and nutrient supplementation. Also provided are compositions useful for carrying out such methods.

### Definitions

As used herein, an “integrating” genetic element refers to a nucleic acid that is incorporated into the genome of a microorganism. A “non-integrating” genetic element is a nucleic acid that is not incorporated into the genome of a microorganism. An integrating element may be incorporated, e.g., into a target gene location, while a non-integrating element may be part of, e.g., a plasmid.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs.

In some embodiments, identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described herein. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega® (Sievers et al., Mol Syst Biol. 2011 Oct. 11; 7:539) may be used. Unless noted otherwise, the term “sequence identity” in the claims refers to sequence identity as calculated by Clustal Omega® using default parameters.

As used herein, a residue (such as a nucleic acid residue or an amino acid residue) in sequence “X” is referred to as corresponding to a position or residue (such as a nucleic acid residue or an amino acid residue) “a” in a different sequence “Y” when the residue in sequence “X” is at the counterpart position of “a” in sequence “Y” when sequences X and Y are aligned using amino acid sequence alignment tools known in the art, such as, for example, Clustal Omega® or BLAST®.

When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). Similarity is a more sensitive measure of relatedness between sequences than identity; it takes into account not only identical (i.e. 100% conserved) residues but also non-identical yet similar (in size, charge, etc.) residues. The exact numerical value for percent similarity can depend on various parameters, such as the substitution matrix employed to calculate it, e.g., BLOSUM45 vs. BLOSUM90.

The term “polypeptide” or “protein” or “peptide” is specifically intended to cover naturally occurring proteins, as well as those which are recombinantly or synthetically produced. It should be noted that the term “polypeptide” or “protein” may include naturally occurring modified forms of the proteins, such as glycosylated forms. The terms “polypeptide” or “protein” or “peptide” as used herein are intended to encompass any amino acid sequence and include modified sequences such as glycoproteins.

As used herein, the terms “cellular organism”, “microorganism”, or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic and non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell

The term “wild-type microorganism” or “wild-type host cell” describes a cell that occurs in nature, i.e. a cell that has not been genetically modified.

The term “genetically engineered” may refer to any manipulation of a host cell's genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells. In other embodiments, a host cell may be a genetically identical cell that lacks a specific gene being tested in the treatment host cell.

### Method of Prototrophic Gene Editing

### Overview of Technology and Benefits

Current methods of CRISPR gene editing require multiple, inefficient rounds of gene editing to introduce and subsequently remove the molecular tools required for editing a target gene of interest. By contrast, the methods of the present disclosure provide novel ways of editing the genome of a host strain without residual extraneous genetic material.

In some embodiments, the present methods accomplish this goal through the strategic manipulation of prototrophy and/or auxotrophy. By integrating a nucleic acid construct into a gene required for prototrophy, the present inventors discovered that gene editing tools could be strategically selected for and then selected against to allow for “loop in” and subsequent “loop out” events without the need for multiple rounds of time-consuming gene editing. In some embodiments, this is accomplished by the use of an integrating nucleic acid construct.

In some embodiments, the integrating nucleic acid construct is complemented by the use of a non-integrating nucleic acid construct that can similarly be selected for and against in subsequent steps of the gene editing process.

Each of these features is described in further detail in the sections herein.

### Prototrophic Gene Selection and Manipulation

The methods of the present disclosure involve the manipulation of host cell prototrophy and/or auxotrophy.

“Prototrophy,” as used herein, refers to the ability of a microorganism to synthesize organic compounds required for its growth. A microorganism may generally be referred to as “prototrophic” if it has the nutritional requirements associated with a wild type strain. Prototrophic cells are self-sufficient producers of required metabolites, e.g., amino acids, lipids, and cofactors. In some contexts herein, prototrophy is specific to a particular nutrient: e.g., a microorganism prototrophic for tryptophan is able to synthesize tryptophan without the need for exogenous supplementation within the growth medium.

By contrast, “auxotrophy,” as used herein, is the inability of an organism to synthesize a particular organic compound required for its growth. Auxotrophs require growth medium supplemented with the metabolite that they cannot synthesize. For example, a methionine auxotrophic cell would require media containing methionine in order to replicate. An organism may be auxotrophic or prototrophic for more than one organic compound. For a given organic compound, replica plating may be employed to distinguish between prototrophic and auxotrophic cells.

The methods of the present disclosure involve strategically manipulating prototrophy and auxotrophy. In some embodiments, a host cell is prototrophic for a particular metabolite and the method of the present disclosure involves transiently disrupting this metabolite-specific prototrophy, resulting in a temporarily auxotrophic host cell. This disruption is accomplished, in some embodiments, by the integration of an integrating nucleic acid construct into a prototrophic gene: i.e., a gene required for prototrophy. After disruption, in some embodiments, prototrophy is restored by host-mediated excision of the integrated nucleic acid construct. In some embodiments, prototrophy is restored by a recombination event that results in loss of the integrated nucleic acid construct or the payload thereof.

In some embodiments, the prototrophic gene is involved in a metabolite biosynthesis pathway. In some embodiments, the metabolite is a primary metabolite. A primary metabolite is any intermediate in, or product of the primary metabolism in cells. The primary metabolism in cells is the sum of metabolic activities that are common to most, if not all, living cells and are necessary for basal growth and maintenance of the cells. Primary metabolism thus includes pathways for generally modifying and synthesizing certain carbohydrates, proteins, fats and nucleic acids, with the compounds involved in the pathways being designated primary metabolites. Primary metabolites are necessary for basal growth and maintenance of the cell and include certain nucleic acids, amino acids, proteins, fats, and carbohydrates. In some embodiments, the metabolite is an amino acid, an alcohol, a nucleotide, an antioxidant, a lipid, a cofactor, a fatty acid, a nutrient, a polyol, a vitamin, an organic acid, or the like. In some embodiments, the metabolite is a secondary metabolite. The term “secondary metabolite” means a compound, derived from primary metabolites, that is produced by an organism, is not a primary metabolite, is not ethanol or a fusel alcohol, and is not required for growth under standard conditions. Secondary metabolites are derived from intermediates of many pathways of primary metabolism. In some embodiments, the production of a secondary metabolite is manipulated in the present methods by exposing the cells to non-standard conditions in which the secondary metabolite is required for growth, such that its manipulation can be used to produce prototrophic/auxotrophic cells.

Different conditions and selection criteria affect the choice of metabolite biosynthesis to manipulate. In some embodiments, the metabolite is one that can be supplemented in a growth medium. In some embodiments, the auxotroph incapable of producing that metabolite grows at the same rate as the prototroph when supplemented with the required nutrient. In some embodiments, the metabolite is commercially available and/or readily supplied externally to the cell. In some embodiments, the required media to supplement the lack of metabolite-prototrophy is known and is implemented within the present methods.

In some embodiments, one or more than one metabolic activity is selected for disruption within the present methods. In some embodiments, the prototrophic gene or metabolite can be of a biosynthetic-type (anabolic), of a utilization-type (catabolic), or may be chosen from both types. For example, in some embodiments, one or more than one activity in a given biosynthetic pathway for the selected metabolite is knocked-out; or more than one activity, each from different biosynthetic pathways, are knocked-out.

Compounds and molecules whose biosynthesis or utilization can be targeted to produce auxotrophic host cells include: lipids, including, for example, fatty acids; mono- and disaccharides and substituted derivatives thereof, including, for example, glucose, fructose, sucrose, glucose-6-phosphate, and glucuronic acid, as well as Entner-Doudoroff and Pentose Phosphate pathway intermediates and products; nucleosides, nucleotides, dinucleotides, including, for example, nitrogenous bases, including, for example, pyridines, purines, pyrimidines, pterins, and hydro-, dehydro-, and/or substituted nitrogenous base derivatives, such as cofactors, for example, biotin, cobamamide, riboflavine, thiamine; organic acids and glycolysis and citric acid cycle intermediates and products, including, for example, hydroxyacids and amino acids.

In some embodiments, the prototrophic gene is involved in the biosynthesis of a metabolite selected from the group consisting of: the lipids; the nucleosides, nucleotides, dinucleotides, nitrogenous bases, and nitrogenous base derivatives; and the organic acids and glycolysis and citric acid cycle intermediates and products. In some embodiments, the prototrophic gene is involved in the biosynthesis of a metabolite selected from the group consisting of: the nucleosides, nucleotides, dinucleotides, nitrogenous bases, and nitrogenous base derivatives; and the organic acids and glycolysis and citric acid cycle intermediates and products. In some embodiments, the prototrophic gene is involved in the biosynthesis of a metabolite selected from the group consisting of: the pyrimidine nucleosides, nucleotides, dinucleotides, nitrogenous bases, and nitrogenous base derivatives; and the amino acids.

In some embodiments, the metabolite is an amino acid and the prototrophic gene is involved in an amino acid biosynthesis pathway. In some embodiments, the amino acid is alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine. In some embodiments, the amino acid is alanine. In some embodiments, the amino acid is arginine. In some embodiments, the amino acid is asparagine. In some embodiments, the amino acid is cysteine. In some embodiments, the amino acid is glutamic acid. In some embodiments, the amino acid is glutamine. In some embodiments, the amino acid is glycine. In some embodiments, the amino acid is histidine. In some embodiments, the amino acid is isoleucine. In some embodiments, the amino acid is leucine. In some embodiments, the amino acid is lysine. In some embodiments, the amino acid is methionine. In some embodiments, the amino acid is phenylalanine. In some embodiments, the amino acid is proline. In some embodiments, the amino acid is serine. In some embodiments, the amino acid is threonine. In some embodiments, the amino acid is tryptophan. In some embodiments, the amino acid is tyrosine. In some embodiments, the amino acid is valine.

In some embodiments, the metabolite is a nucleotide, nucleoside, nucleobase, or analog thereof, and the prototrophic gene is involved in the biosynthesis thereof. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (e.g., guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. In some embodiments, the metabolite is adenine, cytosine, guanine, thymine, or uracil. In some embodiments, the metabolite is adenosine, guanosine, cytidine, thymidine, or uridine. In some embodiments, the metabolite is adenine. In some embodiments, the metabolite is cytosine. In some embodiments, the metabolite is guanine. In some embodiments, the metabolite is thymine. In some embodiments, the metabolite is uracil. In some embodiments, the metabolite is uracil and the prototrophic gene is URA3.

### Integrating Nucleic Acid Construct Design

The present methods involve the use of an integrating nucleic acid construct, e.g., a Removal by Prototrophic Selection (RePS) vector. In some embodiments, the integrating nucleic acid construct is integrated into a prototrophic gene, thereby disrupting host cell prototrophy. In some embodiments, the integrating nucleic acid construct is integrated into the host cell genome via homologous recombination, CRISPR, or another gene editing technique known in the art. In some embodiments, single-crossover homologous recombination is used between a circular plasmid or vector and the host cell genome in order to loop-in the circular plasmid or vector.

In some embodiments, the integrating nucleic acid construct comprises a nucleic acid sequence encoding a gene used to edit the genome of the host cell. In some embodiments, the integrating nucleic acid construct comprises a nucleic acid sequence encoding a selectable or counterselectable marker. In some embodiments, the integrating nucleic acid construct comprises repeat sequences flanking the other components of the construct.

For example, in some embodiments, the integrating nucleic acid construct is a Removal by Prototrophic Selection (RePS) vector. In some embodiments, a RePS vector is used to enable target gene editing and subsequent removal of gene editing tools. RePS vectors are used for genome engineering, resulting in strains comprising the desired gene edits without extraneous genetic alterations from the gene editing process. RePS vectors disrupt the function of a gene required for prototrophy when integrated into the genome. These vectors comprise a payload flanked by repeats that when recombined restore prototrophy for the auxotrophy created by the RePS vector. In the process of restoring the prototrophy, the payload is removed. Since prototrophy can only occur by a gain of function event, the payload can be efficiently and reliably removed by selecting for prototrophs, making RePS vectors useful for high-throughput genome engineering.

Gene-Editing Component

In some embodiments, a component of the integrating nucleic acid construct is a nucleotide sequence encoding a gene-editing protein or gene-editing nucleic acid. In some embodiments, the gene-editing protein or nucleic acid may be a component of a gene editing system. In some embodiments, the gene-editing protein or nucleic acid may be a component of a CRISPR gene editing system, such as any of the components described herein. In some embodiments, the gene-editing protein is a Cas nuclease, such as a Cas9 or Cas12 nuclease.

In some embodiments, the gene-editing protein or gene-editing nucleic acid is one which indirectly leads to genome editing, e.g., through mating. Therefore, in some embodiments, the integrating nucleic acid construct comprises a gene encoding a protein that enables mating between different host strains derived from the same genetic background to combine different genetic edits of interest comprised by different host strains. In some embodiments, the gene enables mating by enabling mating type switching. In some embodiments, the gene encodes the HO endonuclease.

In some embodiments, the gene-editing component is a recombineering system or a component thereof, e.g., for editing prokaryotic genomes. Recombineering was originally based on homologous recombination in Escherichia coli mediated by bacteriophage proteins, either RecE/RecT from Rac prophage or Redαβδ from bacteriophage lambda. Recombineering utilizes linear DNA substrates that are either double-stranded (dsDNA) or single-stranded (ssDNA). In some embodiments, the gene-editing component of the integrating nucleic acid construct comprises one or more of the gam, bet, and exo phage recombination genes of the bacteriophage λ Red system. In some embodiments, the gene-editing component of the integrating nucleic acid construct comprises all three of the gam, bet, and exo phage recombination genes of the bacteriophage λ Red system.

In some embodiments, the gene-editing component is a dominant version of a mutator polymerase that introduces mutations into a genome. In some embodiments, a method employing a dominant mutator polymerase gene would result in mutated host cells, which host cells could then be selected for a desired genotype/phenotype and then, using the tools provided herein, the polymerase would be removed from the genome.

In some embodiments, the gene-editing component is a homing endonuclease, e.g., intron-encoded endonuclease I-SceI. In some embodiments, the I-SceI endonuclease functions within the present methods by making double-strand breaks in the genome of the host cell that are repaired with a donor molecule homologous with the regions flanking the break.

Selectable Markers

In some embodiments, a component of the integrating nucleic acid construct is a nucleotide sequence encoding a selectable marker. In some embodiments, the selectable marker is a dominant selectable marker. In some embodiments, the selectable marker is used to select for host cells comprising the integrating nucleic acid construct.

In some embodiments, the integrating nucleic acid construct comprises a counterselectable marker. In some embodiments, the selectable marker is also a counterselectable marker.

Selectable markers, counterselectable markers, and selection methods are described in detail herein in the section entitled “Selection components and methods” and are suitable for use within the integrating nucleic acid construct in some embodiments.

Repeat Nucleotides and Excision

In some embodiments, a component of the integrating nucleic acid construct is a pair of repeat nucleotide sequences flanking the coding region of the integrating nucleic acid construct. In some embodiments, the repeat nucleotide sequences are 50-1000 nucleotides in length. In some embodiments, the repeat nucleotide sequences are 20-60 nucleotides in length. In some embodiments, the repeat nucleotide sequences are about 10, about 20, about 30, about 40, about 50, about 60, about 70, about 80, about 90, about 100, about 200, about 300, about 400, or about 500 nucleotides in length.

In some embodiments, these repeat nucleotide sequences facilitate excision by mitotic recombination, such that the integrating nucleic acid construct or some component thereof is excised from the host genome. In some embodiments, this occurs after editing of the target gene of interest by selecting for prototrophic host cells. Additional guidance on this process can be found, e.g., in Akada et al., Yeast 2006; 23(5): 399-405, incorporated by reference herein in its entirety, and in the Looping out section as follows.

Looping Out

In some embodiments, the present disclosure teaches methods comprising looping out the integrated nucleic acid construct, or a portion thereof, from the host cell genome. The looping out method can be as described in Nakashima et al. 2014 “Bacterial Cellular Engineering by Genome Editing and Gene Silencing.” Int. J. Mol. Sci. 15(2), 2773-2793, incorporated by reference herein. In some embodiments, the present disclosure teaches looping out the integrated nucleic acid construct, or a portion thereof, from positive transformants. Looping out deletion techniques are known in the art, and are described in Tear et al., “Excision of Unstable Artificial Gene-Specific inverted Repeats Mediates Scar-Free Gene Deletions in Escherichia coli,” Appl Biochem Biotech 2014; 175: 1858-1867, incorporated by reference herein. In some embodiments, the looping out methods used in the methods provided herein are performed using single-crossover homologous recombination or double-crossover homologous recombination. In some embodiments, looping out of selected regions as described herein entails using single-crossover homologous recombination as described herein.

First, integrating nucleic acid constructs are inserted into selected target regions within the genome of the host organism (e.g., via homologous recombination, CRISPR, or other gene editing techniques). In some embodiments, the integrating nucleic acid construct is comprised by a circular plasmid or a vector, and single-crossover homologous recombination is used between the circular plasmid or vector and the host cell genome in order to loop-in the circular plasmid or vector. In some embodiments, the integrating nucleic acid construct comprises a sequence which is a direct repeat of an existing or introduced nearby host sequence, such that the direct repeats flank the region of DNA slated for looping out, i.e., deletion. In some embodiments, once integrated into the genome, cells comprising the integrating nucleic acid construct are subjected to counterselection for deletion of the integrated nucleic acid construct or a portion thereof (e.g., restoration of prototrophy).

### Non-Integrating Nucleic Acid Construct Design

In some embodiments, the disclosed methods make use of non-integrating nucleic acid constructs. In some embodiments, the non-integrating nucleic acid construct comprises a nucleic acid sequence encoding a gene editing protein or gene editing nucleic acid. In some embodiments, the non-integrating nucleic acid construct comprises a selectable marker. In some embodiments, the non-integrating nucleic acid construct complements the auxotrophy induced by the integration of the integrating nucleic acid construct. In some embodiments, the non-integrating nucleic acid construct comprises a nucleotide sequence encoding a gene complementing the function of the prototrophic gene disrupted within the method.

In some embodiments, the non-integrating nucleic acid construct complements the payload comprised by the integrating nucleic acid construct. For example, in some embodiments, the integrating nucleic acid construct comprises a nucleotide sequence encoding an endonuclease, e.g., a Cas nuclease such as Cas9 or Cas12, and the non-integrating nucleic acid construct comprises a nucleotide sequence encoding an sgRNA.

Examples of non-integrating nucleic acid constructs for use within the methods disclosed herein include, without limitation, plasmids, cosmids, mRNA vectors, viruses, and artificial chromosomes, such as bacterial artificial chromosomes (BACs) and P1-derived artificial chromosomes (PACs).

Gene-Editing Component

In some embodiments, a component of the non-integrating nucleic acid construct is a nucleotide sequence encoding a gene-editing protein or gene-editing nucleic acid. In some embodiments, the gene-editing protein or nucleic acid may be a component of a gene editing system. In some embodiments, the gene-editing protein or nucleic acid may be a component of a CRISPR gene editing system, such as any of the components disclosed herein. In some embodiments, the gene-editing nucleic acid is an sgRNA.

In some embodiments, the gene-editing protein or gene-editing nucleic acid is one which indirectly leads to genome editing, e.g., through mating. Therefore, in some embodiments, the non-integrating nucleic acid construct comprises a gene encoding a protein that enables mating between different host strains to combine different genetic edits of interest comprised by different host strains. In some embodiments, the gene enables mating by enabling mating type switching. In some embodiments, the gene encodes the HO endonuclease.

In some embodiments, the gene-editing component is a recombineering system or a component thereof, e.g., for editing prokaryotic genomes. Recombineering utilizes linear DNA substrates that are either double-stranded (dsDNA) or single-stranded (ssDNA). In some embodiments, the gene-editing component of the non-integrating nucleic acid construct comprises the linear DNA substrate for the recombineering system.

In some embodiments, the gene-editing component functions in a method comprising the use of a homing endonuclease, e.g., intron-encoded endonuclease I-SceI. In some embodiments, the gene-editing component of the non-integrating nucleic acid construct is a donor nucleic acid molecule used to repair a double-strand break introduced by the I-SceI endonuclease in the genome of the host cell, wherein the donor nucleic acid molecule is homologous with the regions flanking the break.

Auxotrophy Complementation

In some embodiments, the non-integrating nucleic acid construct comprises a nucleotide sequence encoding a gene that complements the function of the prototrophic gene disrupted by the integration of the integrating nucleic acid construct. In some embodiments, this component of the non-integrating nucleic acid construct cannot recombine with the host cell genome, in order to prevent restoration of prototrophy through an integration event. In some embodiments, this allows for the selection of host cells comprising both the integrated integrating nucleic acid construct and the non-integrating nucleic acid construct. For example, in some embodiments, cells are selected for comprising both constructs through selection for the dominant selectable marker comprised by the integrating nucleic acid construct and through selection for prototrophy complemented by the non-integrating nucleic acid construct.

Selectable Markers

In some embodiments, a component of the non-integrating nucleic acid construct is a nucleotide sequence encoding a selectable marker. In some embodiments, the selectable marker is a dominant selectable marker. In some embodiments, the selectable marker is used to select for host cells comprising the non-integrating nucleic acid construct.

In some embodiments, the non-integrating nucleic acid construct comprises a counterselectable marker. In some embodiments, the selectable marker is also a counterselectable marker.

Selectable markers, counterselectable markers, and selection methods are described in detail herein in the section entitled “Selection components and methods” and are suitable for use within the non-integrating nucleic acid construct in some embodiments.

### Selection Components and Methods

In some embodiments, the integrating nucleic acid constructs, non-integrating nucleic acid constructs, and host cells disclosed herein comprise one or more selectable markers. In some embodiments, the methods disclosed herein comprise selection steps to select for cells that comprise or do not comprise the integrating nucleic acid construct or the non-integrating nucleic acid construct or a component thereof.

Illustrative Selectable Markers

As used herein, the term “selectable marker” refers to a gene which functions as guidance for selecting a host cell comprising an integrating or non-integrating nucleic acid construct as described herein. After transformation within a method disclosed herein, in some embodiments, a given transgenic host cell comprises one or more than one selection marker or selection marker system. For example, one or more biosynthesis selection marker(s) or selection marker system(s) according to the present invention may be used together with each other, and/or may be used in combination with a utilization-type selection marker or selection marker system according to the present disclosure. In some embodiments, in the prototrophy-manipulating embodiments herein, the host cell may also comprise one or more non-auxotrophic selection marker(s) or selection marker system(s).

Selectable markers for use within the present methods and compositions include, but are not limited to: fluorescent markers, luminescent markers, drug selectable markers, prototrophic/auxotrophic markers, and the like.

In some embodiments, the selectable marker is a fluorescent marker or a luminescent marker. Fluorescent markers include, but are not limited to, genes encoding fluorescence proteins such as green fluorescent protein (GFP), cyan fluorescent protein (CFP), yellow fluorescent protein (YFP), red fluorescent protein (dsRFP) and the like. Luminescent markers include, but are not limited to, genes encoding luminescent proteins such as luciferases. In some embodiments, reporter genes, such as the lac Z reporter gene for facilitating blue/white selection of transformed colonies, or fluorescent proteins such as green, red and yellow fluorescent proteins, are used as selectable marker genes to facilitate selection of host cells comprising the integrating nucleic acid construct and/or non-integrating nucleic acid construct. In some embodiments, rather than growing the transformed cells in media containing selective compound, e.g., antibiotic, the cells are grown under conditions sufficient to allow expression of the reporter, and selection can be performed via visual, colorimetric or fluorescent detection of the reporter.

In some embodiments, the selectable marker is a drug selectable marker. A drug selectable marker enables cells to detoxify an exogenous drug that would otherwise kill the cell. Illustrative examples of drug selectable markers include but are not limited to those which confer resistance to antibiotics such as ampicillin, tetracycline, kanamycin, bleomycin, streptomycin, hygromycin, neomycin, Zeocin™, gentamicin, chloramphenicol, and the like. In some embodiments, the drug selectable marker is a toxin-resistant marker gene, such as, for example, imidazolinone-resistant mutants of acetolactate synthase (“ALS;” EC 2.2.1.6) in which mutation(s) are expressed that make the enzyme insensitive to toxin-inhibition exhibited by versions of the enzyme that do not contain such mutation(s). In some embodiments, the drug, toxin, or compound used to exert selective pressure exerts this effect directly. In some embodiments, the drug, toxin, or compound used to exert selective pressure exerts this effect indirectly, for example, as a result of metabolic action of the cell that converts the drug, toxin, or compound into toxic form or as a result of combination of the drug, toxin, or compound with at least one further compound.

Illustrative selectable markers include a bleomycin-resistance gene, a metallothionein gene, a hygromycin B-phosphotransferase gene, the AURI gene, an adenosine deaminase gene, an aminoglycoside phosphotransferase gene, a dihydrofolate reductase gene, a thymidine kinase gene, a xanthine-guanine phosphoribosyltransferase gene, and the like. pBR and pUC-derived plasmids contain as a selectable marker the bacterial drug resistance marker AMP^{τ} or BLA gene (See, Sutcliffe, J. G., et al., Proc. Natl. Acad. Sci. U.S.A. 75:3737 (1978)).

In some embodiments, selectable markers include but are not limited to: NAT1, PAT, AUR1-C, PDR4, SMR1, CAT, mouse dhfr, HPH, DSDA, KAN^{R}, and SHBLE genes. The NAT1 gene of S. noursei encodes nourseothricin N-acetyltransferase and confers resistance to nourseothricin. The PAT gene from S. viridochromogenes Tu94 encodes phosphinothricin N-acetyltransferase and confers resistance to bialophos. The AUR1-C gene from S. cerevisiae confers resistance to Auerobasidin A (AbA), an antifungal antibiotic produced by Aureobasidium pullulans that is toxic to budding yeast S. cerevisiae. The PDR4 gene confers resistance to cerulenin. The SMR1 gene confers resistance to sulfometuron methyl. The CAT coding sequence from Tn9 transposon confers resistance to chloramphenicol. The mouse dhfr gene confers resistance to methotrexate. The HPH gene of Klebsiella pneumonia encodes hygromycin B phosphotransferase and confers resistance to Hygromycin B. The DSDA gene of E. coli encodes D-serine deaminase and allows yeast to grow on plates with D-serine as the sole nitrogen source. The KA/VR gene of the Tn903 transposon encodes aminoglycoside phosphotransferase and confers resistance to G418. The SHBLE gene from Streptoalloteichus hindustanus encodes a Zeocin binding protein and confers resistance to Zeocin (bleomycin).

In some embodiments, the selectable marker is a prototrophic/auxotrophic marker. Prototrophic/auxotrophic markers are as described in the “Prototrophic gene selection and manipulation” section herein, and include the strategic disruption and complementation of prototrophy as a means for selecting host cells comprising the integrating and/or non-integrating nucleic acid constructs.

In some embodiments, the selectable marker is an auxotrophic marker. An auxotrophic marker allows cells to synthesize an essential component (usually an amino acid) while grown in media that lacks that essential component. Selectable auxotrophic gene sequences include, for example, hisD, which allows growth in histidine free media in the presence of histidinol. In some embodiments, the selectable marker rescues a nutritional auxotrophy in the host strain. In such embodiments, the host strain comprises a functional disruption in one or more genes of the amino acid biosynthetic pathways of the host that cause an auxotrophic phenotype, such as, for example, HIS3, LEU2, LYS2, MET15, and TRP1, or a functional disruption in one or more genes of the nucleotide biosynthetic pathways of the host that cause an auxotrophic phenotype, such as, for example, ADE2 and URA3. In particular embodiments, the host cell comprises a functional disruption in the URA3 gene. The functional disruption in the host cell that causes an auxotrophic phenotype can be a point mutation, a partial or complete gene deletion, or an addition or substitution of nucleotides. Functional disruptions within the amino acid or nucleotide biosynthetic pathways cause the host strains to become auxotrophic mutants which, in contrast to the prototrophic wild-type cells, are incapable of optimum growth in media without supplementation with one or more nutrients. The functionally disrupted biosynthesis genes in the host strain can then serve as auxotrophic gene markers which can later be rescued, for example, upon introducing one or more plasmids comprising a functional copy of the disrupted biosynthesis gene.

In yeast, utilization of the URA3, TRP1, and LYS2 genes as selectable markers allows for both positive and negative selections. Positive selection is carried out by auxotrophic complementation of the URA3, TRP1, and LYS2 mutations whereas negative selection is based on the specific inhibitors 5-fluoro-orotic acid (FOA), 5-fluoroanthranilic acid, and a-aminoadipic acid (aAA), respectively, that prevent growth of the prototrophic strains but allow growth of the URA3, TRP1, and LYS2 mutants, respectively. The URA3 gene encodes orotidine-5′phosphate decarboxylase, an enzyme that is required for the biosynthesis of uracil. Ura3−(or ura5−) cells can be selected on media containing FOA, which kills all URA3+ cells but not ura3− cells because FOA appears to be converted to the toxic compound 5-fluorouracil by the action of the decarboxylase. The negative selection on FOA media is highly discriminating, and usually less than 10′ FOA-resistant colonies are Ura+. The FOA selection procedure can be used to produce ura3 markers in haploid strains by mutation, and, more importantly, for selecting those cells that do not have the URA3-containing plasmids. The TRP1 gene encodes a phosphoribosylanthranilate isomerase that catalyzes the third step in tryptophan biosynthesis. Counterselection using 5-fluoroanthranilic acid involves antimetabolism by the strains that lack enzymes required for the conversion of anthranilic acid to tryptophan and thus are resistant to 5-fluroanthranilic acid. The LYS2 gene encodes an aminoadipate reductase, an enzyme that is required for the biosynthesis of lysine. Lys2− and lys5− mutants, but not normal strains, grow on a medium lacking the normal nitrogen source but containing lysine and aAA. These mutations cause the accumulation of a toxic intermediate of lysine biosynthesis that is formed by high levels of aAA, but these mutants still can use aAA as a nitrogen source. Similar with the FOA selection procedure, LYS2− or TRP1− containing plasmids can be conveniently expelled from Lys2 or trp1 hosts, respectively.

In addition to those selectable markers described above, a wide variety of selectable markers are known in the art. See, for example, Kaufinan, Meth. Enzymol., 185:487 (1990); Kaufman, Meth. Enzymol., 185:537 (1990); Srivastava and Schlessinger, Gene, 103:53 (1991); Romanos et al., in DNA Cloning 2: Expression Systems, 2nd Edition, pages 123-167 (IRL Press 1995); Markie, Methods Mol. Biol., 54:359 (1996); Pfeifer et al., Gene, 188:183 (1997); Tucker and Burke, Gene, 199:25 (1997); Hashida-Okado et al., FEBS Letters, 425:117 (1998), the contents of each of which are incorporated by reference herein in their entirety.

In some embodiments, an integrating nucleic acid construct, a non-integrating nucleic acid construct, or a transgenic host cell disclosed herein comprises a selectable marker or a counter-selectable marker, or a selectable and counter-selectable marker, as disclosed in Table 1.

TABLE 1
Exemplary Selectable/Counter-Selectable Markers

| Marker name | Selection | Counterselection |
|---|---|---|
| LYS2 | Lysine dropout | alpha-aminoadipate |
| LYS5 | Lysine dropout | alpha-aminoadipate |
| CAN1 | Arginine dropout | canavanine |
| amdS | Acetamide as nitrogen | fluoroacetamide |
| | source | |
| FCY1 | Cytosine dropout | 5_fluorocytosine |
| FCA1 | Cytosine dropout | 5_fluorocytosine |
| GAP1 | L-citrulline | D-Histidine |
| URA3 | Uracil dropout | 5-FOA |
| HSV_TK | FUdR | antifolate media |
| TRP1 | Tryptophan dropout | 5-fluoroanthranilic |
| | | acid |

Selection Methods

In some embodiments, the present methods include one or more steps used to select or counterselect for expression of a selectable marker.

In some embodiments, the selection may be positive selection; that is, the cells expressing the marker are isolated from a population, e.g. to create an enriched population of cells comprising the selectable marker. In other instances, the selection may be negative selection; that is, the population is isolated away from the cells, e.g. to create an enriched population of cells that do not comprise the selectable marker.

Separation of cells comprising the selectable marker from cells not comprising the selectable marker may be carried out by any convenient separation technique appropriate for the selectable marker used. For example, if a fluorescent marker has been utilized, in some embodiments, cells are separated by fluorescence activated cell sorting, whereas if a cell surface marker has been inserted, in some embodiments, cells are separated from the heterogeneous population by affinity separation techniques, e.g. magnetic separation, affinity chromatography, “panning” with an affinity reagent attached to a solid matrix, or other convenient technique. When prototrophic/auxotrophic markers are used, or when toxin resistance markers are used, in some embodiments, separation is carried out de facto by the survival of the cells under growth conditions in which selective pressure is applied: e.g., the growth medium comprises antibiotics or does not comprise a required metabolite. In some embodiments, when selecting for cells that are auxotrophic for a certain metabolite, sister plates may be used to identify cells that grow in the presence of metabolite supplementation, but do not grow when the metabolite is absent from the medium.

In some embodiments, selection of the desired cells is based on selecting for drug resistance encoded by a selectable marker. Positive selection systems are those that promote the growth of transformed cells. They may be divided into conditional-positive or non-conditional-positive selection systems. A conditional-positive selection system consists of a gene coding for a protein, usually an enzyme, that confers resistance to a specific substrate that is toxic to untransformed cells or that encourages growth and/or differentiation of the transformed cells. In conditional-positive selection systems the substrate may act in one of several ways. It may be an antibiotic, an herbicide, a drug or metabolite analogue, or a carbon supply precursor. In each case, the gene codes for an enzyme with specificity to a substrate to encourage the selective growth and proliferation of the transformed cells. The substrate may be toxic or non-toxic to the untransformed cells. The nptII gene, which confers kanamycin resistance by inhibiting protein synthesis, is a classic example of a system that is toxic to untransformed cells. The manA gene, which codes for phosphomannose isomerase, is an example of a conditional-positive selection system where the selection substrate is not toxic. In this system, the substrate mannose is unable to act as a carbon source for untransformed cells but it will promote the growth of cells transformed with manA. Non-conditional-positive selection systems do not require external substrates yet promote the selective growth and differentiation of transformed cells. An example in plants is the ipt gene that enhances shoot development by modifying the plant hormone levels endogenously.

Negative selection systems result in the death of transformed cells. These are dominant selectable marker systems that may be described as conditional and non-conditional selection systems. When the selection system is not substrate dependent, it is a non-conditional-negative selection system. An example is the expression of a toxic protein, such as a ribonuclease to ablate specific cell types. When the action of the toxic gene requires a substrate to express toxicity, the system is a conditional negative selection system. These include the bacterial codA gene, which codes for cytosine deaminase, the bacterial cytochrome P450 mono-oxygenase gene, the bacterial haloalkane dehalogenase gene, or the Arabidopsis alcohol dehydrogenase gene. Each of these converts non-toxic agents to toxic agents resulting in the death of the transformed cells. The codA gene has also been shown to be an effective dominant negative selection marker for chloroplast transformation. The Agrobacterium aux2 and tms2 genes can also be used in positive selection systems.

Combinations of positive-negative selection systems are useful for the integration methods provided herein, as in some embodiments, positive selection is utilized to enrich for cells that have successfully integrated the integrating nucleic acid construct, and negative selection is used to eliminate the construct from the same population once the desired gene editing has taken place. Similarly, in some embodiments, positive selection is used to select for cells comprising the non-integrating nucleic acid construct and then negative selection is used to select for cells that no longer comprise the non-integrating nucleic acid construct.

A flow cytometric cell sorter can be used to isolate cells positive for expression of fluorescent markers or proteins (e.g., antibodies) coupled to fluorophores and having affinity for the marker protein. In some embodiments, multiple rounds of sorting may be carried out. In one embodiment, the flow cytometric cell sorter is a FACS machine. Other fluorescence plate readers, including those that are compatible with high-throughput screening can also be used. MACS (magnetic cell sorting) can also be used, for example, to select for host cells with proteins coupled to magnetic beads and having affinity for the marker protein. This is especially useful where the selectable marker encodes, for example, a membrane protein, transmembrane protein, membrane anchored protein, cell surface antigen or cell surface receptor (e.g., cytokine receptor, immunoglobulin receptor family member, ligand-gated ion channel, protein kinase receptor, G-protein coupled receptor (GPCR), nuclear hormone receptor and other receptors; CD14 (monocytes), CD56 (natural killer cells), CD335 (NKp46, natural killer cells), CD4 (T helper cells), CD8 (cytotoxic T cells), CD1c (BDCA-1, blood dendritic cell subset), CD303 (BDCA-2), CD304 (BDCA-4, blood dendritic cell subset), NKp80 (natural killer cells, gamma/delta T cells, effector/memory T cells), “6B11” (Va24Nb11; invariant natural killer T cells), CD137 (activated T cells), CD25 (regulatory T cells) or depleted for CD138 (plasma cells), CD4, CD8, CD19, CD25, CD45RA, CD45RO). Thus, in some embodiments, the selectable marker comprises a protein displayed on the host cell surface, which can be readily detected with an antibody, for example, coupled to a fluorophore or to a colorimetric or other visual readout.

### Gene Editing

### DNA Nucleases

In some embodiments, the present disclosure teaches methods of editing a target gene of interest through the use of DNA nucleases. In some embodiments, a nucleotide sequence encoding the DNA nuclease is comprised by the integrating or non-integrating nucleic acid construct. CRISPR complexes, transcription activator-like effector nucleases (TALENs), zinc finger nucleases (ZFNs), and Fold restriction enzymes are some of the sequence-specific nucleases that have been used as gene editing tools and are suitable for use within the present methods and systems. These enzymes are able to target their nuclease activities to desired target loci through interactions with guide regions engineered to recognize sequences of interest. In some embodiments, the present methods employ CRISPR-based gene editing methods through the use of integrating and/or non-integrating nucleic acid constructs comprising nucleotide sequences encoding one or more components of a CRISPR-based system.

The principles of in vivo CRISPR-based editing largely rely on natural cellular DNA repair systems. Double-stranded dsDNA breaks introduced by nucleases are repaired by either non-homologous end-joining (NHEJ) or homology-directed repair (HDR), or single strand annealing, (SSA), or microhomology end joining (MMEJ).

HDR relies on a template DNA containing sequences homologous to the region surrounding the targeted site of DNA cleavage. Cellular repair proteins use the homology between the exogenously supplied or endogenous DNA sequences and the site surrounding a DNA break to repair the dsDNA break, replacing the break with the sequence on the template DNA. Failure to integrate the template DNA however, can result in NHEJ, MMEJ, or SSA. NHEJ, MMEJ and SSA are error-prone processes that are often accompanied by insertion or deletion of nucleotides (indels) at the target site, resulting in genetic knockout (silencing) of the targeted region of the genome due to frameshift mutations or insertions of a premature stop codon.

CRISPR endonucleases are also useful for in vitro DNA manipulations, as discussed in later sections of this disclosure.

CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). See Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321, incorporated by reference herein. Bacteria and archaea possessing one or more CRISPR loci respond to viral or plasmid challenge by integrating short fragments of the foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001:18; 688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are two CRISPR-Cas system classes, classified based on their effector proteins: class 1 systems possess multi-subunit crRNA-effector complexes, whereas in class 2 systems all functions of the effector complex are carried out by a single protein (e.g., Cas9 or Cpf1). In some embodiments, the present disclosure teaches using class 1 CRISPR systems and components thereof, e.g., Cas3 or Cas10 endonucleases.

In some embodiments, the present disclosure teaches using class 2 CRISPR systems. Within class 2, there are at least three types and 17 subtypes. See Makarova, K. S., et al., “Evolutionary classification of CRISPR-Cas systems: a burst of class 2 and derived variants,” Nat. Rev. Microbial. 2019: 1-17, herein incorporated by reference in its entirety. In some embodiments, the present disclosure teaches using class 2 CRISPR-Cas Types II, V, and/or VI single-subunit effector systems within the disclosed methods. In some embodiments, the present disclosure teaches using CRISPR-Cas components of any one of the 17 class 2 subtypes: II-A, II-B, II-C, V-A, V-B, V-C, V-D, V-E, V-F, V-G, V-H, V-I, V-K, VI-A, VI-B, VI-C, and VI-D.

In some embodiments, the methods of the present disclosure teach methods of gene editing using integrating or non-integrating nucleic acid constructs encoding a CRISPR effector protein/endonuclease selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, and c2c10. In some embodiments, the endonuclease for use in the integrating and/or non-integrating nucleic acid constructs of the present disclosure is a Cms1 endonuclease.

CRISPR/Cas9

In some embodiments, the present disclosure teaches methods of gene editing using a Type II CRISPR system with components encoded by genes comprised by the integrating and/or non-integrating nucleic acid constructs disclosed herein. In some embodiments, the Type II CRISPR system uses the Cas9 enzyme. Type II systems rely on a i) single endonuclease protein, ii) a transactiving crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system are replaced by a single-guide RNA (sgRNA). In some embodiments, the sgRNA includes, for example, a nucleotide sequence that comprises an at least 12-20 nucleotide sequence complementary to the target DNA sequence (guide sequence) and a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex employs additional complementary base-pairing with a protospacer adjacent motif (PAM) (e.g., 5′-NGG-3′) located in a 3′ portion of the target DNA, downstream from the target protospacer. See Jinek, M., et. al., Science. 2012:337; 816-821, incorporated by reference herein. In some embodiments, the PAM motif recognized by a Cas9 varies for different Cas9 proteins.

In some embodiments, the Cas9 peptide of the present disclosure includes one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343 (6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein are used with the wild type Cas9 protein having double-stranded nuclease activity, Cas9 mutants that act as single stranded nickases, or other mutants with modified nuclease activity.

CRISPR/Cas12a

In some embodiments, the present disclosure teaches methods of gene editing using a Type V CRISPR system with components encoded by genes comprised by the integrating and/or non-integrating nucleic acid constructs disclosed herein. In some embodiments, the present disclosure teaches methods of using a CRISPR-Cas12 system. In some embodiments, the present disclosure teaches methods of using CRISPR from Prevotella and Francisella 1 (Cpf1, now termed Cas12a).

The Cas12a CRISPR systems of the present disclosure comprise i) a single endonuclease protein, and ii) a crRNA, wherein a portion of the 3′ end of crRNA contains the guide sequence complementary to a target nucleic acid. In this system, the Cas12a nuclease is directly recruited to the target DNA by the crRNA. In some embodiments, guide sequences for Cas12a must be at least 12nt, 13nt, 14nt, 15nt, or 16nt in order to achieve detectable DNA cleavage, and a minimum of 14nt, 15nt, 16nt, 17nt, or 18nt to achieve efficient DNA cleavage.

The Cas12a systems of the present disclosure differ from Cas9 in a variety of ways. First, unlike Cas9, Cas12a does not require a separate tracrRNA for cleavage. In some embodiments, Cas12a crRNAs are as short as about 42-44 bases long—of which 23-25 nt is guide sequence and 19 nt is the constitutive direct repeat sequence. In contrast, in some embodiments, the combined Cas9 tracrRNA and crRNA synthetic sequences are about 100 bases long. In some embodiments, the present disclosure will refer to a crRNA for Cas12a as a “guide RNA.”

Second, Cas12a prefers a “TTTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cas12a are staggered by about 3-5 bases, which create “sticky ends” (Kim et al., 2016. “Genome-wide analysis reveals specificities of Cpf1 endonucleases in human cells” published online Jun. 6, 2016). These sticky ends with −3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA

Fourth, in Cas12a complexes, the “seed” region is located within the first 5 nt of the guide sequence. Cas12a crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771, incorporated by reference herein). Critically, unlike the Cas9 CRISPR target, the cleavage sites and the seed region of Cas12a systems do not overlap. Additional guidance on designing Cas12a crRNA targeting oligos is available in Zetsche B. et al., “Cpf1 Is a Single RNA-Guided Endonuclease of a Class 2 CRISPR-Cas System” Cell 2015; 163: 759-771.

CRISPRi and CRISPRa

In some embodiments, the present methods and systems employ other CRISPR based techniques to further accelerate identification of helpful edits are CRISPR interference (CRISPRi) and CRISPR activation (CRISPRa). Labs have engineered a Cas9 protein variant (called “dead Cas9”, or dCas9), that retains guide RNA and DNA binding but does not cut the genome. In CRISPRi, targeting dCas9 to DNA upstream of the gene causes repression. Similarly, CRISPRa is used to recruit of transcription factors by fusing appropriate protein binding domains to dCas9. Specificity is still conferred by expressing a guide RNA, but no repair DNA is used. In some embodiments, these techniques are used to screen for useful genetic edits, then follow-up strains are built using more robust genome editing approaches.

### Molecular Tools for Gene Editing

As aforementioned, the present disclosure provides methods of gene editing without residual extraneous nucleic acid sequences. In some embodiments, the present methods and systems are supported by a suite of molecular tools, which enable the creation of genetic design libraries and allow for the efficient implementation of multiple genetic alterations into a given host strain. Techniques for programming genetic designs for implementation to host strains are described in pending U.S. patent application Ser. No. 15/140,296, entitled “Microbial Strain Design System and Methods for Improved Large Scale Production of Engineered Nucleotide Sequences,” incorporated by reference in its entirety herein.

In some embodiments, the molecular tool sets utilized in the present methods and systems include: (1) Promoter swaps (PRO Swap), (2) SNP swaps, (3) Start/Stop codon exchanges, (4) STOP swaps, and (5) Sequence optimization. This suite of molecular tools, either in isolation or combination, enables the creation of genetic design host cell libraries.

In some embodiments, various gene editing strategies are employed in the methods and systems of the present disclosure, and some exemplary gene editing tools are briefly discussed herein. Additional details may be found in, e.g., U.S. Pat. No. 9,988,624, the contents of which are incorporated by reference herein in their entirety.

### Cell Culture and Fermentation

In some embodiments, the present disclosure further teaches measuring the phenotypic performance of host cells. In some embodiments, these steps involve the culturing of host cells. In some embodiments, cells of the present disclosure are cultured in conventional nutrient media modified as appropriate for any desired biosynthetic reactions or selections. In some embodiments, the present disclosure teaches culture in inducing media for activating promoters. In some embodiments, the present disclosure teaches media with selection agents, including selection agents of transformants (e.g., antibiotics), or selection of organisms suited to grow under inhibiting conditions (e.g., high ethanol conditions). In some embodiments, the present disclosure teaches growing cell cultures in media optimized for cell growth. In other embodiments, the present disclosure teaches growing cell cultures in media optimized for product yield. In some embodiments, the present disclosure teaches growing cultures in media capable of inducing cell growth and also contains the necessary precursors for final product production (e.g., high levels of sugars for ethanol production).

Culture conditions, such as temperature, pH and the like, are those suitable for use with the host cell selected for expression, and will be apparent to those skilled in the art. As noted, many references are available for the culture and production of many cells, including cells of bacterial, plant, animal (including mammalian) and archaebacterial origin. See e.g., Sambrook, Ausubel (all supra), as well as Berger, Guide to Molecular Cloning Techniques, Methods in Enzymology volume 152 Academic Press, Inc., San Diego, CA; and Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelle et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N. Y.); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue from Sigma-Aldrich, Inc (St Louis, Mo.) (“Sigma-LSRCCC”) and, for example, The Plant Culture Catalogue and supplement also from Sigma-Aldrich, Inc (St Louis, Mo.) (“Sigma-PCCS”), all of which are incorporated herein by reference.

The culture medium to be used must in a suitable manner satisfy the demands of the respective strains. Descriptions of culture media for various microorganisms are present in the “Manual of Methods for General Bacteriology” of the American Society for Bacteriology (Washington D.C., USA, 1981).

The present disclosure furthermore provides a process for fermentative preparation of a product of interest, comprising the steps of: a) culturing a microorganism according to the present disclosure in a suitable medium, resulting in a fermentation broth; and b) concentrating the product of interest in the fermentation broth of a) and/or in the cells of the microorganism.

In some embodiments, the present disclosure teaches that the microorganisms produced are cultured continuously—as described, for example, in WO 05/021772—or discontinuously in a batch process (batch cultivation) or in a fed-batch or repeated fed-batch process for the purpose of producing the desired organic-chemical compound. A summary of a general nature about known cultivation methods is available in the textbook by Chmiel (Bioprozeßtechnik. 1: Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

In some embodiments, the cells of the present disclosure are grown under batch or continuous fermentation conditions.

Classical batch fermentation is a closed system, wherein the compositions of the medium is set at the beginning of the fermentation and is not subject to artificial alternations during the fermentation. A variation of the batch system is a fed-batch fermentation which also finds use in the present disclosure. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. In some embodiments, continuous fermentation generally maintains the cultures at a constant high density where cells are primarily in log phase growth. In some embodiments, continuous fermentation generally maintains the cultures at a stationary or late log/stationary, phase growth. Continuous fermentation systems strive to maintain steady state growth conditions.

Methods for modulating nutrients and growth factors for continuous fermentation processes as well as techniques for maximizing the rate of product formation are well known in the art of industrial microbiology.

For example, a non-limiting list of carbon sources for the cultures of the present disclosure include, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

A non-limiting list of the nitrogen sources for the cultures of the present disclosure include, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. In some embodiments, the nitrogen sources are used individually or as a mixture.

A non-limiting list of the possible phosphorus sources for the cultures of the present disclosure include, phosphoric acid, potassium dihydrogen phosphate or dipotassium hydrogen phosphate or the corresponding sodium-containing salts.

In some embodiments, the culture medium additionally comprises salts, for example in the form of chlorides or sulfates of metals such as, for example, sodium, potassium, magnesium, calcium and iron, such as, for example, magnesium sulfate or iron sulfate, which are necessary for growth.

Finally, in some embodiments, essential growth factors such as amino acids, for example homoserine and vitamins, for example thiamine, biotin or pantothenic acid, are employed in addition to the above mentioned substances.

In some embodiments, the pH of the culture is controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

In some embodiments, the cultures of the present disclosure include an anti-foaming agent such as, for example, fatty acid polyglycol esters. In some embodiments the cultures of the present disclosure are modified to stabilize the plasmids of the cultures by adding suitable selective substances such as, for example, antibiotics.

In some embodiments, the culture is carried out under aerobic conditions. In order to maintain these conditions, oxygen or oxygen-containing gas mixtures such as, for example, air are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation is preferably continued until an amount of the desired product of interest (e.g. an organic-chemical compound) sufficient for being recovered has formed. In some embodiments, this aim is achieved within 10 hours to 160 hours. In continuous processes, longer cultivation times are possible. The activity of the microorganisms results in a concentration (accumulation) of the product of interest in the fermentation medium and/or in the cells of said microorganisms.

In some embodiments, the culture is carried out under anaerobic conditions.

### Product Recovery and Quantification

In some embodiments, the methods of the present disclosure are used to edit host cells for improved production of a product of interest. Methods for screening for the production of products of interest are known to those of skill in the art and are discussed throughout the present specification. In some embodiments, such methods are employed when screening the strains of the disclosure.

In some embodiments, the present disclosure teaches systems and methods for improving or enabling a desired function, such as producing (or increasing the production of) a product of interest. In some embodiments, the present disclosure teaches systems and methods that manufacture host cells with genes that perform the same function as target genes, such as producing (or increasing the production of) a product of interest. In some embodiments, the host cells of the present invention are designed to produce non-secreted intracellular products. For example, the present disclosure teaches methods of improving the robustness, yield, efficiency, or overall desirability of cell cultures producing intracellular enzymes, oils, pharmaceuticals, or other valuable small molecules or peptides. In some embodiments, the recovery or isolation of non-secreted intracellular products is achieved by lysis and recovery techniques that are well known in the art, including those described herein.

For example, in some embodiments, cells of the present disclosure are harvested by centrifugation, filtration, settling, or other method. Harvested cells are then disrupted by any convenient method, including freeze-thaw cycling, sonication, mechanical disruption, or use of cell lysing agents, or other methods, which are well known to those skilled in the art.

In some embodiments, the resulting product of interest, e.g. a polypeptide, is recovered/isolated and optionally purified by any of a number of methods known in the art. For example, in some embodiments, a product polypeptide is isolated from the nutrient medium by conventional procedures including, but not limited to: centrifugation, filtration, extraction, spray-drying, evaporation, chromatography (e.g., ion exchange, affinity, hydrophobic interaction, chromatofocusing, and size exclusion), or precipitation. Finally, in some embodiments, high performance liquid chromatography (HPLC) is employed in the final purification steps. (See for example Purification of intracellular protein as described in Parry et al., 2001, Biochem. 1353:117, and Hong et al., 2007, Appl. Microbiol. Biotechnol. 73:1331, both incorporated herein by reference).

In addition to the references noted supra, a variety of purification methods are well known in the art, including, for example, those set forth in: Sandana (1997) Bioseparation of Proteins, Academic Press, Inc.; Bollag et al. (1996) Protein Methods, 2^{nd} Edition, Wiley-Liss, NY; Walker (1996) The Protein Protocols Handbook Humana Press, NJ; Harris and Angal (1990) Protein Purification Applications: A Practical Approach, IRL Press at Oxford, Oxford, England; Harris and Angal Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference.

In some embodiments, the present disclosure teaches host cells designed to produce secreted products. For example, the present disclosure teaches methods of improving the robustness, yield, efficiency, or overall desirability of cell cultures producing valuable small molecules or peptides.

In some embodiments, immunological methods are used to detect and/or purify secreted or non-secreted products produced by the cells of the present disclosure. In one example approach, antibody raised against a product molecule (e.g., against an insulin polypeptide or an immunogenic fragment thereof) using conventional methods is immobilized on beads, mixed with cell culture media under conditions in which the endoglucanase is bound, and precipitated. In some embodiments, the present disclosure teaches the use of enzyme-linked immunosorbent assays (ELISA).

In other related embodiments, immunochromatography is used, as disclosed in U.S. Pat. Nos. 5,591,645, 4,855,240, 4,435,504, 4,980,298, and Se-Hwan Paek, et al., “Development of rapid One-Step Immunochromatographic assay, Methods”, 22, 53-60, 2000), each of which are incorporated by reference herein. A general immunochromatography detects a specimen by using two antibodies. A first antibody exists in a test solution or at a portion at an end of a test piece in an approximately rectangular shape made from a porous membrane, where the test solution is dropped. This antibody is labeled with latex particles or gold colloidal particles (this antibody will be called as a labeled antibody hereinafter). When the dropped test solution includes a specimen to be detected, the labeled antibody recognizes the specimen so as to be bonded with the specimen. A complex of the specimen and labeled antibody flows by capillarity toward an absorber, which is made from a filter paper and attached to an end opposite to the end having included the labeled antibody. During the flow, the complex of the specimen and labeled antibody is recognized and caught by a second antibody (it will be called as a tapping antibody hereinafter) existing at the middle of the porous membrane and, as a result of this, the complex appears at a detection part on the porous membrane as a visible signal and is detected.

In some embodiments, the screening methods of the present disclosure are based on photometric detection techniques (absorption, fluorescence). For example, in some embodiments, detection is based on the presence of a fluorophore detector such as GFP bound to an antibody. In some embodiments, the photometric detection is based on the accumulation on the desired product from the cell culture. In some embodiments, the product is detectable via UV of the culture or extracts from said culture.

Persons having skill in the art will recognize that the methods of the present disclosure are compatible with host cells producing any desirable biomolecule product of interest. Table 2 below presents a non-limiting list of the product categories, biomolecules, and host cells, included within the scope of the present disclosure. These examples are provided for illustrative purposes, and are not meant to limit the applicability of the presently disclosed technology in any way.

TABLE 2
A non-limiting list of the host cells and products of interest of the present disclosure.

| Product category | Products | Host category | Hosts |
|---|---|---|---|
| Amino acids | Lysine | Bacteria | Corynebacterium glutamicum |
| Amino acids | Methionine | Bacteria | Escherichia coli |
| Amino acids | MSG | Bacteria | Corynebacterium glutamicum |
| Amino acids | Threonine | Bacteria | Escherichia coli |
| Amino acids | Threonine | Bacteria | Corynebacterium glutamicum |
| Amino acids | Tryptophan | Bacteria | Corynebacterium glutamicum |
| Enzymes | Enzymes (11) | Filamentous fungi | Trichoderma reesei |
| Enzymes | Enzymes (11) | Fungi | Myceliopthora thermophila |
| | | | (C1) |
| Enzymes | Enzymes (11) | Filamentous | Aspergillus oryzae |
| | | fungi | |
| Enzymes | Enzymes (11) | Filamentous | Aspergillus niger |
| | | fungi | |
| Enzymes | Enzymes (11) | Bacteria | Bacillus subtilis |
| Enzymes | Enzymes (11) | Bacteria | Bacillus licheniformis |
| Enzymes | Enzymes (11) | Bacteria | Bacillus clausii |
| Flavor & | Agarwood | Yeast | Saccharomyces cerevisiae |
| Fragrance | | | |
| Flavor & | Ambrox | Yeast | Saccharomyces cerevisiae |
| Fragrance | | | |
| Flavor & | Nootkatone | Yeast | Saccharomyces cerevisiae |
| Fragrance | | | |
| Flavor & | Patchouli | Yeast | Saccharomyces cerevisiae |
| Fragrance | oil | | |
| Flavor & | Saffron | Yeast | Saccharomyces cerevisiae |
| Fragrance | | | |
| Flavor & | Sandalwood | Yeast | Saccharomyces cerevisiae |
| Fragrance | oil | | |
| Flavor & | Valencene | Yeast | Saccharomyces cerevisiae |
| Fragrance | | | |
| Flavor & | Vanillin | Yeast | Saccharomyces cerevisiae |
| Fragrance | | | |
| Food | CoQ10/ | Yeast | Schizosaccharomyces pombe |
| | Ubiquinol | | |
| Food | Omega 3 fatty | Microalgae | Schizochytrium |
| | acids | | |
| Food | Omega 6 fatty | Microalgae | Schizochytrium |
| | acids | | |
| Food | Vitamin B12 | Bacteria | Propionibacterium freudenreichii |
| Food | Vitamin B2 | Filamentous | Ashbya gossypii |
| | | fungi | |
| Food | Vitamin B2 | Bacteria | Bacillus subtilis |
| Food | Erythritol | Yeast-like | Torula coralline |
| | | fungi | |
| Food | Erythritol | Yeast-like | Pseudozyma tsukubaensis |
| | | fungi | |
| Food | Erythritol | Yeast-like | Moniliella pollinis |
| | | fungi | |
| Food | Steviol | Yeast | Saccharomyces cerevisiae |
| | glycosides | | |
| Hydrocolloids | Diutan gum | Bacteria | Sphingomonas sp |
| Hydrocolloids | Gellan gum | Bacteria | Sphingomonas elodea |
| Hydrocolloids | Xanthan gum | Bacteria | Xanthomonas campestris |
| Intermediates | 1,3-PDO | Bacteria | Escherichia coli |
| Intermediates | 1,4-BDO | Bacteria | Escherichia coli |
| Intermediates | Butadiene | Bacteria | Cupriavidus necator |
| Intermediates | n-butanol | Bacteria | Clostridium acetobutylicum |
| | | (obligate | |
| | | anaerobe) | |
| Organic acids | Citric acid | Filamentous | Aspergillus niger |
| | | fungi | |
| Organic acids | Citric acid | Yeast | Pichia guilliermondii |
| Organic acids | Gluconic acid | Filamentous | Aspergillus niger |
| | | fungi | |
| Organic acids | Itaconic acid | Filamentous | Aspergillus terreus |
| | | fungi | |
| Organic acids | Lactic acid | Bacteria | Lactobacillus |
| Organic acids | Lactic acid | Bacteria | Geobacillus thermoglucosidasius |
| Organic acids | LCDAs - DDDA | Yeast | Candida |
| Polyketides/Ag | Spinosad | Yeast | Saccharopolyspora spinosa |
| Polyketides/Ag | Spinetoram | Yeast | Saccharopolyspora spinosa |

In some embodiments, the molecule of interest is a protein. In some embodiments, the molecule of interest is a metabolite. In some embodiments, the molecule of interest is an amino acid. In some embodiments, the molecule of interest is a vitamin. In some embodiments, the molecule of interest is a commodity chemical. Numerous chemicals are known to be produced or known to be possible to produce in biological culture, such as ethanol, acetone, citric acid, propanoic acid, fumaric acid, butanol and 2,3-butanediol. See, e.g., Saxena, “Microbes in Production of Commodity Chemicals,” Applied Microbiology 2015: 71-81, incorporated by reference herein in its entirety. In some embodiments, the molecule of interest is a fine chemical. In some embodiments, the molecule of interest is a specialty chemical. In some embodiments, the molecule of interest is a pharmaceutical. In some embodiments, the molecule of interest is a biofuel. In some embodiments, the molecule of interest is a biopolymer.

In some embodiments, molecules of interest include alcohols such as ethanol, propanol, isopropanol, butanol, fatty alcohols, fatty acid esters, wax esters; hydrocarbons and alkanes such as propane, octane, diesel, JP8; polymers such as terephthalate, 1,3-propanediol, 1,4-butanediol, polyols, PHA, PHB, acrylate, adipic acid, ε-caprolactone, isoprene, caprolactam, rubber; commodity chemicals such as lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-ADCA/cephalosporin, erythromycin, polyketides, statins, paclitaxel, docetaxel, terpenes, peptides, steroids, omega fatty acids and other such suitable molecules of interest. In some embodiments, such molecules are useful in the context of fuels, biofuels, industrial and specialty chemicals, additives, as intermediates used to make additional products, such as nutritional supplements, nutraceuticals, polymers, paraffin replacements, personal care products and pharmaceuticals. In some embodiments, molecules are used as feedstock for subsequent reactions for example transesterification, hydrogenation, catalytic cracking via either hydrogenation, pyrolisis, or both or epoxidations reactions to make other products.

### Selection Criteria and Goals (Desired Function)

In some embodiments, the present disclosure teaches methods and systems for transient protein and/or gene expression. In some embodiments, this transient expression is for the purpose of improving or enabling a desired function in a host cell. In some embodiments, this transient expression is for the purpose of gene editing in order to improve or enable a desired function in a host cell. As used herein, the term “desired function” refers to the goal of the strain improvement program. In some embodiments the terms “desired function” and “program goal(s)” are used interchangeably in this document.

The selection criteria applied to the methods of the present disclosure will vary with the specific goals of the strain improvement program (i.e., with the desired function that is being enabled or improved). In some embodiments, the present disclosure is adapted to meet any program goals. For example, in some embodiments, the program goal is to maximize single batch yields of reactions with no immediate time limits. In other embodiments, the program goal is to rebalance biosynthetic yields to produce a specific product, or to produce a particular ratio of products. In other embodiments, the program goal is to modify the chemical structure of a product, such as lengthening the carbon chain of a polymer. In some embodiments, the program goal is to improve performance characteristics such as yield, titer, productivity, by-product elimination, tolerance to process excursions, optimal growth temperature and growth rate. In some embodiments, the program goal is improved host performance as measured by volumetric productivity, specific productivity, yield or titer, of a product of interest produced by a microbe.

In some embodiments, the program goal is to identify variants of a target protein or target gene that are improved in at least one respect. In some embodiments, these variants perform the same function or a similar function with one or more improved attributes. For example, in some embodiments, the variant is more catalytically efficient, more pH- or thermo-stable, insensitive to feedback-inhibition or dependent on a different cofactor to catalyze a desired reaction. In some embodiments, the variant is fused with another protein thus enabling more efficient catalysis. In some embodiments, the program goal is to improve characteristics of the target protein, target gene, or production of the target molecule of interest. In some embodiments, the goal is to improve resilience to stress factors. In some embodiments, the stress factor is selected from pH, temperature, osmotic pressure, substrate concentration, product concentration, and byproduct concentration.

In other embodiments, the program goal is to optimize synthesis efficiency of a commercial strain in terms of final product yield per quantity of inputs (e.g., total amount of ethanol produced per pound of sucrose). In other embodiments, the program goal is to optimize synthesis speed, as measured for example in terms of batch completion rates, or yield rates in continuous culturing systems. In other embodiments, the program goal is to increase strain resistance to a particular phage, or otherwise increase strain vigor/robustness under culture conditions.

In some embodiments, strain improvement projects are subject to more than one goal. In some embodiments, the goal of the strain project hinges on quality, reliability, or overall profitability. In some embodiments, the present disclosure teaches methods of associated selected mutations or groups of mutations with one or more of the strain properties described above.

Persons having ordinary skill in the art will recognize how to tailor strain selection criteria to meet the particular project goal. For example, in some embodiments, selections of a strain's single batch max yield at reaction saturation is appropriate for identifying strains with high single batch yields. In some embodiments, selection based on consistency in yield across a range of temperatures and conditions is appropriate for identifying strains with increased robustness and reliability.

In some embodiments, the selection criteria for the initial high-throughput phase and the tank-based validation will be identical. In other embodiments, tank-based selection operates under additional and/or different selection criteria. For example, in some embodiments, high-throughput strain selection is based on single batch reaction completion yields, while tank-based selection is expanded to include selections based on yields for reaction speed.

### Organisms Amenable to Genetic Design

In some embodiments, the present disclosure teaches systems and methods of transient protein and/or gene expression. The disclosed systems and methods of this application are applicable to any host cell organism that is amenable to genetic transformation.

Thus, as used herein, the terms “host cell,” “microbe,” and “microorganism” should be taken broadly. These include, but are not limited to, cells from the two prokaryotic domains, Bacteria and Archaea, as well as certain eukaryotic fungi and protists. However, in some embodiments, “higher” eukaryotic organisms such as insects, plants, and animals are utilized in the methods taught herein.

Suitable host cells include, but are not limited to: bacterial cells, algal cells, plant cells, fungal cells, insect cells, and mammalian cells. In one illustrative embodiment, suitable host cells include E. coli (e.g., SHuffle™ competent E. coli available from New England BioLabs in Ipswich, Mass.).

Other suitable host organisms of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell. Suitable eukaryotic host cells include, but are not limited to: fungal cells, algal cells, insect cells, animal cells, and plant cells. Suitable fungal host cells include, but are not limited to: Ascomycota, Basidiomycota, Deuteromycota, Zygomycota, Fungi imperfecti. Certain preferred fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8th edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell is a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger Group. In an embodiment, the filamentous fungus is Aspergillus niger.

In another embodiment, specific mutants of the fungal species are used for the methods and systems provided herein. In one embodiment, specific mutants of the fungal species are used which are suitable for the high-throughput and/or automated methods and systems provided herein. Examples of such mutants include strains that protoplast very well; strains that produce mainly or, more preferably, only protoplasts with a single nucleus; strains that regenerate efficiently in microtiter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In some embodiments, a specific mutant strain for use in the methods and systems provided herein is a strain lacking a selectable marker gene such as, for example, uridine-requiring mutant strains. In some embodiments, these mutant strains are either deficient in orotidine 5 phosphate decarboxylase (OMPD) or orotate p-ribosyl transferase (OPRT) encoded by the pyrG or pyrE gene, respectively (T. Goosen et al., Curr Genet. 1987, 11:499 503; J. Begueret et al., Gene. 1984 32:487 92.

In one embodiment, specific mutant strains for use in the methods and systems provided herein are strains that possess a compact cellular morphology characterized by shorter hyphae and a more yeast-like appearance.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica. In some embodiments, the host cell is Saccharomyces cerevisiae. In some embodiments, the host cell is Pichia pastoris.

In certain embodiments, the host cell is an algal cell such as, Chlamydomonas (e.g., C. reinhardtii) and Phormidium (P. sp. ATCC29409).

In other embodiments, the host cell is a prokaryotic cell. Suitable prokaryotic cells include gram positive, gram negative, and gram-variable bacterial cells. In some embodiments, the host cell is a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is Corynebacterium glutamicum.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell is an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

In various embodiments, strains that are used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

### Genomic Automation

Automation of the methods of the present disclosure enables high-throughput phenotypic screening and identification of target products from multiple test strain variants simultaneously.

The aforementioned genomic engineering platform, in some embodiments, involves hundreds and thousands of mutant strains constructed in a high-throughput fashion. In some embodiments, the robotic and computer systems described below are the structural mechanisms by which such a high-throughput process is carried out.

In some embodiments, the present disclosure teaches methods of transient protein and/or gene expression. In some embodiments, the methods and systems of the present disclosure comprise manufacturing steps of host cells comprising genetic alterations. In some embodiments, the methods and systems further comprise methods of measuring phenotypic performance of manufactured cells. As part of this process, the present disclosure teaches methods of assembling DNA, building new strains, screening cultures in plates, and screening cultures in models for tank fermentation. In some embodiments, the present disclosure teaches that one or more of the aforementioned methods and systems of creating and testing new host strains is aided by automated robotics.

### HTP Robotic Systems

In some embodiments, the automated methods of the disclosure comprise a robotic system. In some embodiments, the systems outlined herein are generally directed to the use of 96- or 384-well microtiter plates, but as will be appreciated by those in the art, any number of different plates or configurations may be used. In addition, in some embodiments, any or all of the steps outlined herein are automated; thus, for example, in some embodiments, the systems are completely or partially automated.

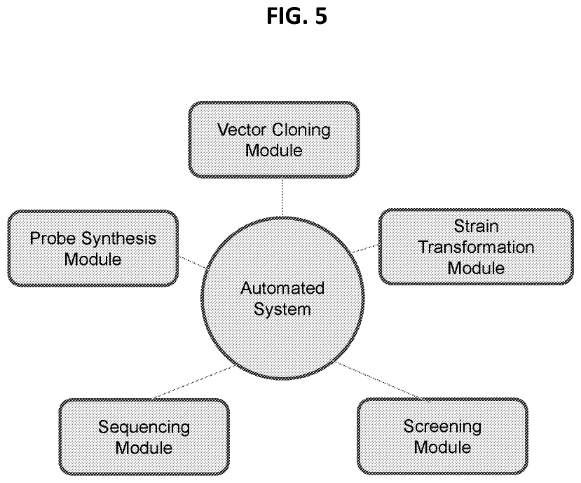

In some embodiments, the automated systems of the present disclosure comprise one or more work modules. For example, in some embodiments, the automated system of the present disclosure comprises a DNA synthesis module, a vector cloning module, a strain transformation module, a screening module, and a sequencing module (see FIG. 5).

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

In some embodiments, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, microarray chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems. In some embodiments, the high-throughput electroporation systems are capable of transforming cells in 96 or 384-well plates. In some embodiments, the high-throughput electroporation systems include VWR® High-throughput Electroporation Systems, BTX™, Bio-Rad® Gene Pulser MXcell™ or other multi-well electroporation systems.

In some embodiments, the integrated thermal cycler and/or thermal regulators are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipettors, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

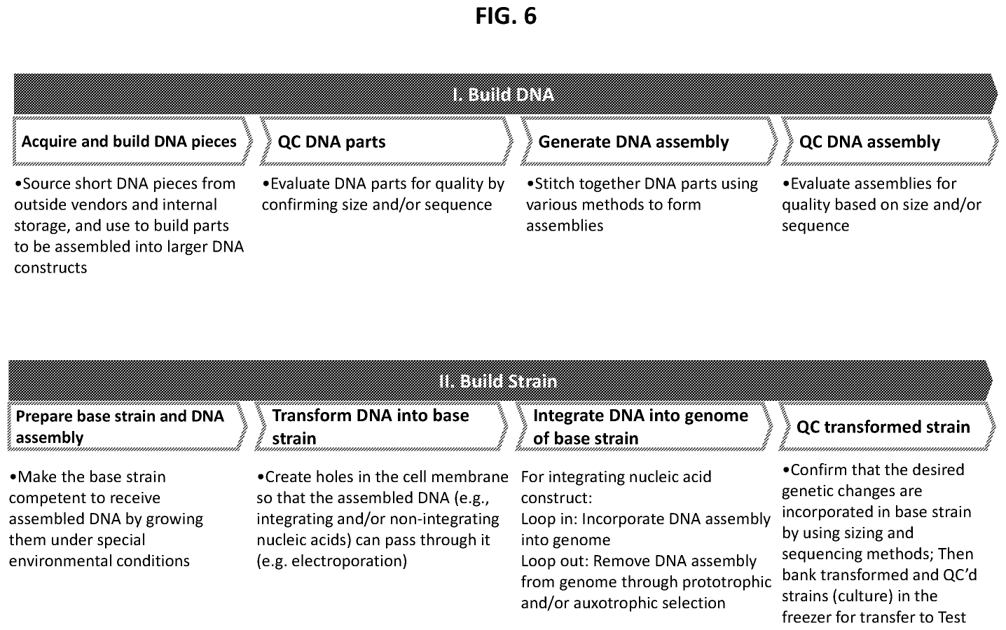

Thus, in some embodiments, the present disclosure teaches a high-throughput strain engineering platform, as depicted in FIG. 6.

Persons having skill in the art will recognize the various robotic platforms capable of carrying out the HTP engineering methods of the present disclosure. Table 3 below provides a non-exclusive list of scientific equipment capable of carrying out each step of the HTP engineering steps of the present disclosure as described in FIG. 6.

TABLE 3
Non-exclusive list of Scientific Equipment Compatible with
the HTP engineering methods of the present disclosure.

| | Equipment | | Compatible Equipment |
|---|---|---|---|
| | Type | Operation(s) performed | Make/Model/Configuration |
| Acquire and | liquid | Hitpicking (combining by | Hamilton Microlab STAR, |
| build DNA | handlers | transferring) primers/ | Labcyte Echo 550, Tecan EVO |
| fragments | | templates for PCR amplification | 200, Beckman Coulter Biomek |
| | | of DNA parts | FX, or equivalents |
| | Thermal | PCR amplification of DNA parts | Inheco Cycler, ABI 2720, ABI |
| | cyclers | | Proflex 384, ABI Veriti, or |
| | | | equivalents |
| QC DNA | Fragment | gel electrophoresis to confirm | Agilent Bioanalyzer, AATI |
| parts | analyzers | PCR products of appropriate size | Fragment Analyzer, or |
| | (capillary | | equivalents |
| | electrophoresis) | | |
| | Sequencer | Verifying sequence of parts/ | Beckman Ceq-8000, Beckman |
| | (Sanger: | templates | GenomeLab ™, or equivalents |
| | Beckman) | | |
| | NGS (next | Verifying sequence of parts/ | Illumina MiSeq series sequences, |
| | generation | templates | illumina Hi-Seq, Ion torrent, pac |
| | sequencing) | | bio or other equivalents |
| | instrument | | |
| | nanodrop/plate | assessing concentration of | Molecular Devices SpectraMax M5, |
| | reader | DNA samples | Tecan M1000, or equivalents. |
| Generate DNA | liquid | Hitpicking (combining by | Hamilton Microlab STAR, Labcyte |
| assembly | handlers | transferring) DNA parts for | Echo 550, Tecan EVO 200, Beckman |
| | | assembly along with cloning | Coulter Biomek FX, or equivalents |
| | | vector, addition of reagents | |
| | | for assembly reaction/process | |
| QC DNA | Colony | for inoculating colonies in | Scirobotics Pickolo, Molecular |
| assembly | pickers | liquid media | Devices QPix 420 |
| | liquid | Hitpicking primers/templates, | Hamilton Microlab STAR, Labcyte |
| | handlers | diluting samples | Echo 550, Tecan EVO 200, Beckman |
| | | | Coulter Biomek FX, or equivalents |
| | Fragment | gel electrophoresis to | Agilent Bioanalyzer, AATI |
| | analyzers | confirm assembled products | Fragment Analyzer |
| | (capillary | of appropriate size | |
| | electrophoresis) | | |
| | Sequencer | Verifying sequence of | ABI3730 Thermo Fisher, Beckman |
| | (sanger: | assembled plasmids | Ceq-8000, Beckman GenomeLab ™, |
| | Beckman) | | or equivalents |
| | NGS (next | Verifying sequence of | Illumina MiSeq series sequences, |
| | generation | assembled plasmids | illumina Hi-Seq, Ion torrent, pac |
| | sequencing) | | bio or other equivalents |
| | instrument | | |
| Prepare base | centrifuge | spinning/pelleting cells | Beckman Avanti floor centrifuge, |
| strain and | | | Hettich Centrifuge |
| DNA assembly | | | |
| Transform DNA | Electroporators | electroporative transformation | BTX Gemini X2, BIO-RAD |
| into base strain | | of cells | MicroPulser Electroporator |
| | Ballistic | ballistic transformation | BIO-RAD PDS1000 |
| | transformation | of cells | |
| | Incubators, | for chemical | Inheco Cycler, ABI 2720, ABI |
| | thermal | transformation/heat shock | Proflex 384, ABI Veriti, or |
| | cyclers | | equivalents |
| | Liquid | for combining DNA, cells, | Hamilton Microlab STAR, Labcyte |
| | handlers | buffer | Echo 550, Tecan EVO 200, Beckman |
| | | | Coulter Biomek FX, or equivalents |
| Integrate DNA | Colony | for inoculating colonies in | Scirobotics Pickolo, Molecular |
| into genome of | pickers | liquid media | Devices QPix 420 |
| base strain | Liquid | For transferring cells onto | Hamilton Microlab STAR, Labcyte |
| | handlers | Agar, transferring from | Echo 550, Tecan EVO 200, Beckman |
| | | culture plates to different | Coulter Biomek FX, or equivalents |
| | | culture plates (inoculation | |
| | | into other selective media) | |
| | Platform shaker- | incubation with shaking of | Kuhner Shaker ISF4-X, Infors-ht |
| | incubators | microtiter plate cultures | Multitron Pro |
| QC transformed | Colony | for inoculating colonies in | Scirobotics Pickolo, Molecular |
| strain | pickers | liquid media | Devices QPix 420 |
| | liquid | Hitpicking primers/templates, | Hamilton Microlab STAR, Labcyte |
| | handlers | diluting samples | Echo 550, Tecan EVO 200, Beckman |
| | | | Coulter Biomek FX, or equivalents |
| | Thermal | cPCR verification of strains | Inheco Cycler, ABI 2720, ABI |
| | cyclers | | Proflex 384, ABI Veriti, or |
| | | | equivalents |
| | Fragment | gel electrophoresis to | Infors-ht Multitron Pro, Kuhner |
| | analyzers | confirm cPCR products of | Shaker ISF4-X |
| | (capillary | appropriate size | |
| | electrophoresis) | | |
| | Sequencer | Sequence verification of | Beckman Ceq-8000, Beckman |
| | (sanger: | introduced modification | GenomeLab ™, or equivalents |
| | Beckman) | | |
| | NGS (next | Sequence verification of | Illumina MiSeq series sequences, |
| | generation | introduced modification | illumina Hi-Seq, Ion torrent, pac |
| | sequencing) | | bio or other equivalents |
| | instrument | | |
| Select and | Liquid | For transferring from | Hamilton Microlab STAR, Labcyte |
| consolidate | handlers | culture plates to different | Echo 550, Tecan EVO 200, Beckman |
| QC'd strains | | culture plates (inoculation | Coulter Biomek FX, or equivalents |
| into test | | into production media) | |
| plate | Colony | for inoculating colonies in | Scirobotics Pickolo, Molecular |
| | pickers | liquid media | Devices QPix 420 |
| | Platform shaker- | incubation with shaking of | Kuhner Shaker ISF4-X, Infors-ht |
| | incubators | microtiter plate cultures | Multitron Pro |
| Culture | Liquid handlers | For transferring from | Hamilton Microlab STAR, Labcyte |
| strains in | | culture plates to different | Echo 550, Tecan EVO 200, Beckman |
| seed plates | | culture plates (inoculation | Coulter Biomek FX, or equivalents |
| | | into production media) | |
| | Platform shaker- | incubation with shaking of | Kuhner Shaker ISF4-X, Infors-ht |
| | incubators | microtiter plate cultures | Multitron Pro |
| | liquid | Dispense liquid culture media | Well mate (Thermo), Benchcel2R |
| | dispensers | into microtiter plates | (velocity 11), plateloc (velocity 11) |
| | microplate | apply barcoders to plates | Microplate labeler (a2 + cab − |
| | labeler | | agilent), benchcell 6R (velocity 11) |
| Generate | Liquid | For transferring from | Hamilton Microlab STAR, Labcyte |
| product from | handlers | culture plates to different | Echo 550, Tecan EVO 200, Beckman |
| strain | | culture plates (inoculation | Coulter Biomek FX, or equivalents |
| | | into production media) | |
| | Platform shaker- | incubation with shaking of | Kuhner Shaker ISF4-X, Infors-ht |
| | incubators | microtiter plate cultures | Multitron Pro |
| | liquid | Dispense liquid culture | well mate (Thermo), Benchcel2R |
| | dispensers | media into multiple microtiter | (velocity 11), plateloc (velocity 11) |
| | | plates and seal plates | |
| | microplate | Apply barcodes to plates | microplate labeler (a2 + cab − |
| | labeler | | agilent), benchcell 6R (velocity 11) |
| Evaluate | Liquid | For processing culture broth | Hamilton Microlab STAR, Labcyte |
| performance | handlers | for downstream analytical | Echo 550, Tecan EVO 200, Beckman |
| | | | Coulter Biomek FX, or equivalents |
| | UHPLC, | quantitative analysis of | Agilent 1290 Series UHPLC and |
| | HPLC | precursor and target | 1200 Series HPLC with UV and RI |
| | | compounds | detectors, or equivalent; also |
| | | | any LC/MS |
| | LC/MS | highly specific analysis of | Agilent 6490 QQQ and 6550 QTOF |
| | | precursor and target compounds | coupled to 1290 Series UHPLC |
| | | as well as side and degradation | |
| | | products | |
| | Spectrophotometer | Quantification of different | Tecan M1000, spectramax M5, |
| | | compounds using | Genesys 10S |
| | | spectrophotometer based assays | |
| Culture | Fermenters: | incubation with shaking | Sartorius, DASGIPs (Eppendorf), |
| strains in | | | BIO-FLOs (Sartorius-stedim). |
| flasks | | | Applikon |
| | Platform | | innova 4900, or any equivalent |
| | shakers | | |
| Generate product | Fermenters: DASGIPs (Eppendorf), BIO-FLOs (Sartorius-stedim) | | |
| from strain | | | |
| Evaluate | Liquid | For transferring from | Hamilton Microlab STAR, Labcyte |
| performance | handlers | culture plates to different | Echo 550, Tecan EVO 200, Beckman |
| | | culture plates (inoculation | Coulter Biomek FX, or equivalents |
| | | into production media) | |
| | UHPLC, | quantitative analysis of | Agilent 1290 Series UHPLC and |
| | HPLC | precursor and target | 1200 Series HPLC with UV and RI |
| | | compounds | detectors, or equivalent; also |
| | | | any LC/MS |
| | LC/MS | highly specific analysis | Agilent 6490 QQQ and 6550 QTOF |
| | | of precursor and target | coupled to 1290 Series UHPLC |
| | | compounds as well as side | |
| | | and degradation products | |
| | Flow | Characterize strain performance | BD Accuri, Millipore Guava |
| | cytometer | (measure viability) | |
| | Spectrophotometer | Characterize strain performance | Tecan M1000, Spectramax M5, or |
| | | (measure biomass) | other equivalents |

### Exemplary Sequences of the Disclosure

The present disclosure provides integrating and non-integrating nucleic acid constructs for use in the disclosed gene-editing methods. Table 4 below provides illustrative sequences of various components for use in the present nucleic acid constructs, and illustrative sequences of both integrating and non-integrating nucleic acid constructs. Any one or more of these sequences are suitable for use in the methods and compositions of the present disclosure.

TABLE 4
Illustrative sequences of the disclosure

| SEQ | | |
|---|---|---|
| ID | | |
| NO | Description | Sequence |
| 1 | pBREW0 | gatttcggtttccttgaaatttttttgattcggtaatctccgaacagaaggaagaacgaaggaaggagcacagacttagatt |
| | RePS- | ggtatatatacgcatatgtagtgttgaagaaacatgaaattgcccagtattcttaacccaactgcacagaacaaaaacctg |
| | Hyg-URA3- | caggaaacgaagataaatcatgtcgaaagctacatataaggaacgtgctgctactcatcctagtcctgttgctgccaagc |
| | Cas9 | tatttaatatcatgcacgaaaagcaaacaaacttgtgtgcttcattggatgttcgtaccaccaaggaattactggagttagtt |
| | | gaagcattaggtcccaaaatttgtttactaaaaacacatgtggatatcttgactgatttttccatggagggcacagttaagc |
| | | cgctaaaggcattatccgccaagtacaattttttactcttcgaagacagaaaatttgctgacattggtaatacagtcaaattg |
| | | cagtactctgcgggtgtatacagaatagcagaatgggcagacattacgaatgcacacggtgtggtgggcccaggtatt |
| | | gttagcggtttgaagcaggcggcagaagaagtaacaaaggaacctagaggccttttgatgttagcagaattgtcatgca |
| | | agggctccctatctactggagaatatactaagggtactgttgacattgcgaagagcgacaaagattttgttatcggctttat |
| | | tgctcaaagagacatgggtgaaggcaagcccagaaaaatatcgcaagcacctttggtcttacagtgccaacttttggcc |
| | | tgccgacgttaagagtacaaagctgatggcaatgtacgacaagataacagagtctcaaaagaagtgaaacaatttttctt |
| | | caccacattttccattgttccttccccccataactataaacgtatttatgtatatatatttgcgtgtaagtgtgtgtactataggg |
| | | caccgtaaagtaataatgcttaattagttactactatgaccatataagaggtcatactgtatgaagccacaaagcagatag |
| | | atcaatcatgtttaacgaaaactgttaatcgaagattatttctttttttttttctctttcctttttacaaagaaaattttttttgcgc |
| | | tttttgccatcaccatcgcaagttctgggacaattgttctctttcgctccagttccaaggaaagaggtttctgttttacttaatagaa |
| | | agtgtcatcttgtattttatatctcttctttcttgtgtaaaattctttagttttgattttgtatttttaggacagtgagctacgaagt |
| | | aacatttttacttaataaccgtttgaagcatagagcaggccctggtaccaccacctaatatctggctttttattcaataaaaactc |
| | | aaaaaaaaaaatccaaaaaaaactaaaaaaccaataaaaataaaatggataagaagtactccatcggtttggatattggt |
| | | actaactccgtcggttgggctgttattactgacgaatacaaagtcccatctaagaagttcaaggtcttgggtaacactgat |
| | | agacactccattaagaagaacttaattggtgccttgttattcgattctggtgaaactgccgaagccactcgtttgaagaga |
| | | actgctagaagacgttacactagaagaaagaaccgtatttgttacttgcaagaaattttctctaatgaaatggctaaagtcg |
| | | acgattccttcttccacagattggaagaatcttttttggtcgaagaagataaaaagcacgaaagacacccaattttcggta |
| | | atattgtcgatgaagtcgcttaccacgaaaagtacccaactatctaccacttgagaaaaaaattggtcgactctaccgata |
| | | aggccgacttaagattgatttacttggctttggcccacatgattaagttcagaggtcactttttgattgaaggtgatttgaac |
| | | ccagataactccgatgttgataaattattcatccaattagtccaaacttataaccaattgttcgaagaaaacccaatcaacg |
| | | cttctggtgtcgatgctaaggctattttgtccgctagattgtctaagtctcgtagattggaaaacttgattgctcaattgccag |
| | | gtgaaaagaagaacggtttgttcggtaacttgattgctttgtccttgggtttgactccaaacttcaagtccaacttcgacttg |
| | | gctgaggatgctaagttacaattatctaaagatacctacgacgatgatttggacaacttattggctcaaattggtgatcaat |
| | | acgccgatttgttcttagccgctaagaacttgtctgacgctattttgttgtctgacattttgagagttaacactgaaatcacca |
| | | aagctccattgtccgcttccatgattaaaagatacgacgaacaccaccaagacttaaccttgttgaaggctttggttagac |
| | | aacaattgccagaaaagtacaaagaaattttcttcgatcaatctaaaaacggttatgccggttacatcgacggtggtgcct |
| | | ctcaagaagaattctataagttcattaagcctatcttggaaaagatggatggtactgaagaattgttagttaagttgaacag |
| | | agaagacttgttgcgtaagcaaagaacctttgacaacggttctatccctcaccaaatccacttgggtgaattgcacgctat |
| | | cttgagaagacaagaggacttctacccattcttaaaggataacagagaaaagatcgaaaaaattttgactttcagaattcc |
| | | atattacgtcggtccattggccagaggtaattctagattcgcttggatgactagaaagtctgaagaaactatcactccatg |
| | | gaatttcgaagaagtcgttgataagggtgcttccgctcaatctttcattgaacgtatgactaacttcgacaaaaatttgccta |
| | | acgaaaaggttttgccaaagcactccttgttgtacgaatattttactgtttacaacgaattgactaaggttaagtacgttacc |
| | | gaaggtatgagaaagccagctttcttgtctggtgaacaaaagaaggccattgttgatttgttgtttaagaccaacagaaag |
| | | gttactgtcaagcaattgaaagaagattacttcaagaagatcgaatgtttcgattctgtcgagatctccggtgttgaggata |
| | | gatttaacgcttctttaggtacctaccacgatttattgaagatcatcaaggacaaggatttcttggacaacgaagaaaacg |
| | | ccacttgttcgatgacaaggtcatgaagcaattgaaaagaagaagatacaccggttggggtagattatccagaaagtta |
| | | attaacggtatcagagataagcaatctggtaagaccatcttggatttcttgaagtccgatggtttcgctaacagaaacttca |
| | | tgcaattgattcatgacgactccttgaccttcaaggaagacattcaaaaagctcaagtttccggtcaaggtgactctttgca |
| | | tgaacacatcgctaacttggccggttccccagctattaagaagggtattttgcaaaccgttaaggtcgtcgacgaattagt |
| | | taaggtcatgggtagacacaagccagaaaacattgttattgaaatggctagagaaaatcaaaccactcaaaaaggtcaa |
| | | aaaaactccagagaaagaatgaagagaattgaagaaggtattaaagaattgggttcccaaattttaaaggaacacccag |
| | | ttgaaaatactcaattacaaaacgagaagttgtatttgtactatttacaaaacggtagagatatgtacgtcgaccaagaatt |
| | | ggttttaactcgttctgacaagaatagaggtaaatccgataacgttccatccgaagaagtcgtcaaaaagatgaaaaact |
| | | actggagacaattgttgaacgctaagttgatcacccaaagaaaatttgataacttaactaaggctgaacgtggtggtttgt |
| | | ccgaattggacaaggccggtttcattaagagacaattagttgaaacccgtcaaattactaagcacgttgctcaaattttgg |
| | | attcccgtatgaacactaagtacgatgaaaacgacaagttgattagagaggtcaaggttattaccttgaagtccaagttgg |
| | | tttccgacttcagaaaggattttcaattttacaaagttcgtgaaatcaacaactatcaccacgctcacgatgcttacttaaac |
| | | gccgttgtcggtaccgctttgattaaaaagtatccaaagttggaatccgaattcgtctacggtgactacaaggtctacgac |
| | | gtcagaaaaatgattgctaagtctgaacaagaaattggtaaggctactgctaagtacttcttctattccaacatcatgaactt |
| | | ttttaaaaccgaaatcaccttggctaatggtgaaatcagaaaaagacctttgatcgaaactaacggtgaaaccggtgaaa |
| | | ttgtttgggataagggtagagacttcgctaccgttagaaaggttttgtccatgccacaagtcaacattgtcaagaagaccg |
| | | aagttcaaaccggtggtttctctaaggaatctatcttgccaaagcgtaattctgacaaattgattgccagaaagaaggatt |
| | | gggatccaaaaaaatacggtggtttcgattctccaactgttgcttactccgtcttggtcgtcgctaaagtcgaaaagggta |
| | | agtctaagaagttgaaatccgtcaaagaattgttgggtatcactattatggaaagatcttctttcgaaaagaacccaatcga |
| | | tttcttggaagccaagggttacaaggaagttaaaaaggacttgatcattaaattgccaaaatactctttgttcgaattggag |
| | | aatggtagaaaaagaatgttggcttccgctggtgaattgcaaaagggtaacgaattggctttgccatccaagtacgttaa |
| | | ctttttgtacttagcctctcactacgaaaagttgaaaggttccccagaagataacgaacaaaaacaattgttcgtcgaaca |
| | | acataaacattatttggatgagattattgaacaaatttctgagttttctaagagagtcatcttggccgacgctaacttggataa |
| | | ggtcttgtccgcctacaacaagcacagagacaagccaatcagagaacaagccgaaaacatcattcacttgttcacttta |
| | | actaacttgggtgccccagctgctttcaaatacttcgacactaccattgacagaaagagatacacttctactaaggaagtc |
| | | ttggatgctactttgatccaccaatccatcactggtttgtacgaaactagaattgatttgtctcaattgggtggtgatagcag |
| | | ggctgaccccaagaagaagaggaaggtgtagtatataactgtctagaaataaacacccgtcgagcctgtccgatttcaa |
| | | agtccccgccgggtcacccggccagcgacatggaggcccagaataccctccttgacagtcttgacgtgcgcagctca |
| | | ggggcatgatgtgactgtcgcccgtacatttagcccatacatccccatgtataatcatttgcatccatacattttgatggcc |
| | | gcacggcgcgaagcaaaaattacggctcctcgctgcagacctgcgagcagggaaacgctcccctcacagacgcgtt |
| | | gaattgtccccacgccgcgcccctgtagagaaatataaaaggttaggatttgccactgaggttcttctttcatatacttcctt |
| | | ttaaaatcttgctaggatacagttctcacatcacatccgaacataaacaaccatgggtaaaaagcctgaactcaccgcga |
| | | cgtctgtcgagaagtttctgatcgaaaagttcgacagcgtctccgacctgatgcagctctcggagggcgaagaatctcg |
| | | tgctttcagcttcgatgtaggagggcgtggatatgtcctgcgggtaaatagctgcgccgatggtttctacaaagatcgtta |
| | | tgtttatcggcactttgcatcggccgcgctcccgattccggaagtgcttgacattggggaattcagcgagagcctgacct |
| | | attgcatctcccgccgtgcacagggtgtcacgttgcaagacctgcctgaaaccgaactgcccgctgttctgcagccggt |
| | | cgcggaggccatggatgcgatcgctgcggccgatcttagccagacgagcgggttcggcccattcggaccgcaagga |
| | | atcggtcaatacactacatggcgtgatttcatatgcgcgattgctgatccccatgtgtatcactggcaaactgtgatggac |
| | | gacaccgtcagtgcgtccgtcgcgcaggctctcgatgagctgatgctttgggccgaggactgccccgaagtccggca |
| | | cctcgtgcacgcggatttcggctccaacaatgtcctgacggacaatggccgcataacagcggtcattgactggagcga |
| | | ggogatgttcggggattcccaatacgaggtcgccaacatcttcttctggaggccgtggttggcttgtatggagcagcag |
| | | acgcgctacttcgagcggaggcatccggagcttgcaggatcgccgcggctccgggcgtatatgctccgcattggtctt |
| | | gaccaactctatcagagcttggttgacggcaatttcgatgatgcagcttgggcgcagggtcgatgcgacgcaatcgtcc |
| | | gatccggagccgggactgtcgggcgtacacaaatcgcccgcagaagcgcggccgtctggaccgatggctgtgtaga |
| | | agtactcgccgatagtggaaaccgacgccccagcactcgtccgagggcaaaggaataatcagtactgacaataaaaa |
| | | gattcttgttttcaagaacttgtcatttgtatagtttttttatattgtagttgttctattttaatcaaatgttagcgtgatttatat |
| | | tttttttcgcctcgacatcatctgcccagatgcgaagttaagtgcgcagaaagtaatatcatgcgtcaatcgtatgtgaatgctgg |
| | | tcgctatactgctgtcgattcgatactaacgccgccatccagtgtcgaaaacgagctcgaattcatcgatgaaagacaga |
| | | aaatttgctgacattggtaatacagtcaaattgcagtactctgcgggtgtatacagaatagcagaatgggcagacattac |
| | | gaatgcacacggtgtggtgggcccaggtattgttagcggtttgaagcaggcggcagaagaagtaacaaaggaaccta |
| | | gaggccttttgatgttagcagaattgtcatgcaagggctccctatctactggagaatatactaagggtactgttgacattgc |
| | | gaagagcgacaaagattttgttatcggctttattgctcaaagagacatgggtggaagagatgaaggttacgattggttgat |
| | | tatgacacccggtgtgggtttagatgacaagggagacgcattgggtcaacagtatagaaccgtggatgatgtggtctct |
| | | acaggatctgacattattattgttggaagaggactatttgcaaagggaagggatgctaaggtagagggtgaacgttaca |
| | | gaaaagcaggctgggaagcatatttgagaagatgcggccagcaaaactaaaaaactgtattataagtaaatgcatgtat |
| | | actaaactcacaaattagagcttcaatttaattatatcagttattacccggg |
| 2 | Removal | gatttcggtttccttgaaatttttttgattcggtaatctccgaacagaaggaagaacgaaggaaggagcacagacttagatt |
| | by | ggtatatatacgcatatgtagtgttgaagaaacatgaaattgcccagtattcttaacccaactgcacagaacaaaaacctg |
| | Proto- | caggaaacgaagataaatcatgtcgaaagctacatataaggaacgtgctgctactcatcctagtcctgttgctgccaagc |
| | trophic | tatttaatatcatgcacgaaaagcaaacaaacttgtgtgcttcattggatgttcgtaccaccaaggaattactggagttagtt |
| | Selection | gaagcattaggtcccaaaatttgtttactaaaaacacatgtggatatcttgactgatttttccatggagggcacagttaagc |
| | (RePS) | cgctaaaggcattatccgccaagtacaattttttactcttcgaagacagaaaatttgctgacattggtaatacagtcaaattg |
| | vector | cagtactctgcgggtgtatacagaatagcagaatgggcagacattacgaatgcacacggtgtggtgggcccaggtatt |
| | RePS-A | gttagcggtttgaagcaggcggcagaagaagtaacaaaggaacctagaggccttttgatgttagcagaattgtcatgca |
| | sequence | agggctccctatctactggagaatatactaagggtactgttgacattgcgaagagcgacaaagattttgttatcggctttat |
| | | tgctcaaagagacatgggtgaaggcaagcccagaaaaatatcgcaagcacctttggtcttacagtgccaacttttggcc |
| | | tgccgacgttaagagtacaaagctgatggcaatgtacgacaagataacagagtctcaaaagaagtgaaacaatttttctt |
| | | caccacattttccattgttccttccccccataactataaacgtatttatgtatatatatttgcgtgtaagtgtgtgtactataggg |
| | | caccgtaaagtaataatgcttaattagttactactatgaccatataagaggtcatactgtatgaagccacaaagcagatag |
| | | atcaatcatgtttaacgaaaactgttaatcgaagattatttctttttttttttctctttcctttttacaaagaaaattttttttgcg |
| | | ctttttgccatcaccatcgcaagttctgggacaattgttctctttcgctccagttccaaggaaagaggtttctgttttacttaatag |
| | | aaagtgtcatcttgtattttatatctcttctttcttgtgtaaaattctttagttttgattttgtatttttaggacagtgagctacga |
| | | agtaacatttttacttaataaccgtttgaagcatagagcaggccctggtaccaccacctaatatctggctttttattcaataaaaac |
| | | tcaaaaaaaaaaatccaaaaaaaactaaaaaaccaataaaaataaaatggataagaagtactccatcggtttggatattggt |
| | | actaactccgtcggttgggctgttattactgacgaatacaaagtcccatctaagaagttcaaggtcttgggtaacactgat |
| | | agacactccattaagaagaacttaattggtgccttgttattcgattctggtgaaactgccgaagccactcgtttgaagaga |
| | | actgctagaagacgttacactagaagaaagaaccgtatttgttacttgcaagaaattttctctaatgaaatggctaaagtcg |
| | | acgattccttcttccacagattggaagaatcttttttggtcgaagaagataaaaagcacgaaagacacccaattttcggta |
| | | atattgtcgatgaagtcgcttaccacgaaaagtacccaactatctaccacttgagaaaaaaattggtcgactctaccgata |
| | | aggccgacttaagattgatttacttggctttggcccacatgattaagttcagaggtcactttttgattgaaggtgatttgaac |
| | | ccagataactccgatgttgataaattattcatccaattagtccaaacttataaccaattgttcgaagaaaacccaatcaacg |
| | | cttctggtgtcgatgctaaggctattttgtccgctagattgtctaagtctcgtagattggaaaacttgattgctcaattgccag |
| | | gtgaaaagaagaacggtttgttcggtaacttgattgctttgtccttgggtttgactccaaacttcaagtccaacttcgacttg |
| | | gctgaggatgctaagttacaattatctaaagatacctacgacgatgatttggacaacttattggctcaaattggtgatcaat |
| | | acgccgatttgttcttagccgctaagaacttgtctgacgctattttgttgtctgacattttgagagttaacactgaaatcacca |
| | | aagctccattgtccgcttccatgattaaaagatacgacgaacaccaccaagacttaaccttgttgaaggctttggttagac |
| | | aacaattgccagaaaagtacaaagaaattttcttcgatcaatctaaaaacggttatgccggttacatcgacggtggtgcct |
| | | ctcaagaagaattctataagttcattaagcctatcttggaaaagatggatggtactgaagaattgttagttaagttgaacag |
| | | agaagacttgttgcgtaagcaaagaacctttgacaacggttctatccctcaccaaatccacttgggtgaattgcacgctat |
| | | cttgagaagacaagaggacttctacccattcttaaaggataacagagaaaagatcgaaaaaattttgactttcagaattcc |
| | | atattacgtcggtccattggccagaggtaattctagattcgcttggatgactagaaagtctgaagaaactatcactccatg |
| | | gaatttcgaagaagtogttgataagggtgcttccgctcaatctttcattgaacgtatgactaacttcgacaaaaatttgccta |
| | | acgaaaaggttttgccaaagcactccttgttgtacgaatattttactgtttacaacgaattgactaaggttaagtacgttacc |
| | | gaaggtatgagaaagccagctttcttgtctggtgaacaaaagaaggccattgttgatttgttgtttaagaccaacagaaag |
| | | gttactgtcaagcaattgaaagaagattacttcaagaagatcgaatgtttcgattctgtcgagatctccggtgttgaggata |
| | | gatttaacgcttctttaggtacctaccacgatttattgaagatcatcaaggacaaggatttcttggacaacgaagaaaacg |
| | | aagatatcttggaagacattgtcttgactttaaccttatttgaagatagagaaatgattgaagaaagattgaagacctacgc |
| | | ccacttgttcgatgacaaggtcatgaagcaattgaaaagaagaagatacaccggttggggtagattatccagaaagtta |
| | | attaacggtatcagagataagcaatctggtaagaccatcttggatttcttgaagtccgatggtttcgctaacagaaacttca |
| | | tgcaattgattcatgacgactccttgaccttcaaggaagacattcaaaaagctcaagtttccggtcaaggtgactctttgca |
| | | tgaacacatcgctaacttggccggttccccagctattaagaagggtattttgcaaaccgttaaggtcgtcgacgaattagt |
| | | taaggtcatgggtagacacaagccagaaaacattgttattgaaatggctagagaaaatcaaaccactcaaaaaggtcaa |
| | | aaaaactccagagaaagaatgaagagaattgaagaaggtattaaagaattgggttcccaaattttaaaggaacacccag |
| | | ttgaaaatactcaattacaaaacgagaagttgtatttgtactatttacaaaacggtagagatatgtacgtcgaccaagaatt |
| | | actggagacaattgttgaacgctaagttgatcacccaaagaaaatttgataacttaactaaggctgaacgtggtggtttgt |
| | | ccgaattggacaaggccggtttcattaagagacaattagttgaaacccgtcaaattactaagcacgttgctcaaattttgg |
| | | attcccgtatgaacactaagtacgatgaaaacgacaagttgattagagaggtcaaggttattaccttgaagtccaagttgg |
| | | tttccgacttcagaaaggattttcaattttacaaagttcgtgaaatcaacaactatcaccacgctcacgatgcttacttaaac |
| | | gccgttgtcggtaccgctttgattaaaaagtatccaaagttggaatccgaattcgtctacggtgactacaaggtctacgac |
| | | gtcagaaaaatgattgctaagtctgaacaagaaattggtaaggctactgctaagtacttcttctattccaacatcatgaactt |
| | | ttttaaaaccgaaatcaccttggctaatggtgaaatcagaaaaagacctttgatcgaaactaacggtgaaaccggtgaaa |
| | | ttgtttgggataagggtagagacttcgctaccgttagaaaggttttgtccatgccacaagtcaacattgtcaagaagaccg |
| | | aagttcaaaccggtggtttctctaaggaatctatcttgccaaagcgtaattctgacaaattgattgccagaaagaaggatt |
| | | gggatccaaaaaaatacggtggtttcgattctccaactgttgcttactccgtcttggtcgtcgctaaagtcgaaaagggta |
| | | agtctaagaagttgaaatccgtcaaagaattgttgggtatcactattatggaaagatcttctttcgaaaagaacccaatcga |
| | | tttcttggaagccaagggttacaaggaagttaaaaaggacttgatcattaaattgccaaaatactctttgttcgaattggag |
| | | aatggtagaaaaagaatgttggcttccgctggtgaattgcaaaagggtaacgaattggctttgccatccaagtacgttaa |
| | | ctttttgtacttagcctctcactacgaaaagttgaaaggttccccagaagataacgaacaaaaacaattgttcgtcgaaca |
| | | acataaacattatttggatgagattattgaacaaatttctgagttttctaagagagtcatcttggccgacgctaacttggataa |
| | | ggtcttgtccgcctacaacaagcacagagacaagccaatcagagaacaagccgaaaacatcattcacttgttcacttta |
| | | actaacttgggtgccccagctgctttcaaatacttcgacactaccattgacagaaagagatacacttctactaaggaagtc |
| | | ttggatgctactttgatccaccaatccatcactggtttgtacgaaactagaattgatttgtctcaattgggtggtgatagcag |
| | | ggctgaccccaagaagaagaggaaggtgtagtatataactgtctagaaataaacacccgtcgagcctgtccgatttcaa |
| | | agtccccgccgggtcacccggccagcgacatggaggcccagaataccctccttgacagtcttgacgtgcgcagctca |
| | | ggggcatgatgtgactgtcgcccgtacatttagcccatacatccccatgtataatcatttgcatccatacattttgatggcc |
| | | gcacggcgcgaagcaaaaattacggctcctcgctgcagacctgcgagcagggaaacgctcccctcacagacgcgtt |
| | | gaattgtccccacgccgcgcccctgtagagaaatataaaaggttaggatttgccactgaggttcttctttcatatacttcctt |
| | | ttaaaatcttgctaggatacagttctcacatcacatccgaacataaacaaccatgggtaaaaagcctgaactcaccgcga |
| | | cgtctgtcgagaagtttctgatcgaaaagttcgacagcgtctccgacctgatgcagctctcggagggcgaagaatctcg |
| | | tgctttcagcttcgatgtaggagggcgtggatatgtcctgcgggtaaatagctgcgccgatggtttctacaaagatcgtta |
| | | tgtttatcggcactttgcatcggccgcgctcccgattccggaagtgcttgacattggggaattcagcgagagcctgacct |
| | | attgcatctcccgccgtgcacagggtgtcacgttgcaagacctgcctgaaaccgaactgcccgctgttctgcagccggt |
| | | cgcggaggccatggatgcgatcgctgcggccgatcttagccagacgagcgggttcggcccattcggaccgcaagga |
| | | atcggtcaatacactacatggcgtgatttcatatgcgcgattgctgatccccatgtgtatcactggcaaactgtgatggac |
| | | gacaccgtcagtgcgtccgtcgcgcaggctctcgatgagctgatgctttgggccgaggactgccccgaagtccggca |
| | | cctcgtgcacgcggatttcggctccaacaatgtcctgacggacaatggccgcataacagcggtcattgactggagcga |
| | | ggogatgttcggggattcccaatacgaggtcgccaacatcttcttctggaggccgtggttggcttgtatggagcagcag |
| | | acgcgctacttcgagcggaggcatccggagcttgcag |
| 3 | promoter | gatttcggtttccttgaaatttttttgattcggtaatctccgaacagaaggaagaacgaaggaaggagcacagacttagatt |
| | pURA3 | ggtatatatacgcatatgtagtgttgaagaaacatgaaattgcccagtattcttaacccaactgcacagaacaaaaacctg |
| | | caggaaacgaagataaatc |
| 4 | URA3 | atgtcgaaagctacatataaggaacgtgctgctactcatcctagtcctgttgctgccaagctatttaatatcatgcacgaaa |
| | flanking | agcaaacaaacttgtgtgcttcattggatgttcgtaccaccaaggaattactggagttagttgaagcattaggtcccaaaa |
| | sequence | tttgtttactaaaaacacatgtggatatcttgactgatttttccatggagggcacagttaagccgctaaaggcattatccgcc |
| | 1 | aagtacaattttttactcttcgaagacagaaaatttgctgacattggtaatacagtcaaattgcagtactctgcgggtgtata |
| | | cagaatagcagaatgggcagacattacgaatgcacacggtgtggtgggcccaggtattgttagcggtttgaagcaggc |
| | | ggcagaagaagtaacaaaggaacctagaggccttttgatgttagcagaattgtcatgcaagggctccctatctactgga |
| | | gaatatactaagggtactgttgacattgcgaagagcgacaaagattttgttatcggctttattgctcaaagagacatgggt |
| | | g |
| 5 | direct | aagacagaaaatttgctgacattggtaatacagtcaaattgcagtactctgcgggtgtatacagaatagcagaatgggca |
| | repeat | gacattacgaatgcacacggtgtggtgggcccaggtattgttagcggtttgaagcaggcggcagaagaagtaacaaa |
| | loop- | ggaacctagaggccttttgatgttagcagaattgtcatgcaagggctccctatctactggagaatatactaagggtactgt |
| | out | tgacattgcgaagagcgacaaagattttgttatcggctttattgctcaaagagacatgggt |
| 6 | promoter | aaggcaagcccagaaaaatatcgcaagcacctttggtcttacagtgccaacttttggcctgccgacgttaagagtacaa |
| | pPAB1 | agctgatggcaatgtacgacaagataacagagtctcaaaagaagtgaaacaatttttcttcaccacattttccattgttcctt |
| | | ccccccataactataaacgtatttatgtatatatatttgcgtgtaagtgtgtgtactatagggcaccgtaaagtaataatgctt |
| | | aattagttactactatgaccatataagaggtcatactgtatgaagccacaaagcagatagatcaatcatgtttaacgaaaa |
| | | ctgttaatcgaagattatttctttttttttttctctttcctttttacaaagaaaattttttttgcgctttttgccatcaccatcg |
| | | caagttctgggacaattgttctctttcgctccagttccaaggaaagaggtttctgttttacttaatagaaagtgtcatcttgtat |
| | | tttatatctcttctttcttgtgtaaaattctttagttttgattttgtatttttaggacagtgagctacgaagtaacatttttactta |
| | | ataaccgtttgaagcatagagcaggccctggtaccaccacctaatatctggctttttattcaataaaaactcaaaaaaaaaaatcca |
| | | aaaaaaactaaaaaaccaataaaaataaa |
| 7 | Cas9 | atggataagaagtactccatcggtttggatattggtactaactccgtcggttgggctgttattactgacgaatacaaagtcc |
| | coding | catctaagaagttcaaggtcttgggtaacactgatagacactccattaagaagaacttaattggtgccttgttattcgattct |
| | sequence | ggtgaaactgccgaagccactcgtttgaagagaactgctagaagacgttacactagaagaaagaaccgtatttgttactt |
| | | gcaagaaattttctctaatgaaatggctaaagtcgacgattccttcttccacagattggaagaatcttttttggtcgaagaag |
| | | ataaaaagcacgaaagacacccaattttcggtaatattgtcgatgaagtcgcttaccacgaaaagtacccaactatctac |
| | | cacttgagaaaaaaattggtcgactctaccgataaggccgacttaagattgatttacttggctttggcccacatgattaagt |
| | | tcagaggtcactttttgattgaaggtgatttgaacccagataactccgatgttgataaattattcatccaattagtccaaactt |
| | | ataaccaattgttcgaagaaaacccaatcaacgcttctggtgtcgatgctaaggctattttgtccgctagattgtctaagtct |
| | | cgtagattggaaaacttgattgctcaattgccaggtgaaaagaagaacggtttgttcggtaacttgattgctttgtccttgg |
| | | gtttgactccaaacttcaagtccaacttcgacttggctgaggatgctaagttacaattatctaaagatacctacgacgatga |
| | | tttggacaacttattggctcaaattggtgatcaatacgccgatttgttcttagccgctaagaacttgtctgacgctattttgttg |
| | | tctgacattttgagagttaacactgaaatcaccaaagctccattgtccgcttccatgattaaaagatacgacgaacaccac |
| | | caagacttaaccttgttgaaggctttggttagacaacaattgccagaaaagtacaaagaaattttcttcgatcaatctaaaa |
| | | acggttatgccggttacatcgacggtggtgcctctcaagaagaattctataagttcattaagcctatcttggaaaagatgg |
| | | atggtactgaagaattgttagttaagttgaacagagaagacttgttgcgtaagcaaagaacctttgacaacggttctatcc |
| | | ctcaccaaatccacttgggtgaattgcacgctatcttgagaagacaagaggacttctacccattcttaaaggataacaga |
| | | gaaaagatcgaaaaaattttgactttcagaattccatattacgtcggtccattggccagaggtaattctagattcgcttggat |
| | | gactagaaagtctgaagaaactatcactccatggaatttcgaagaagtcgttgataagggtgcttccgctcaatctttcatt |
| | | gaacgtatgactaacttcgacaaaaatttgcctaacgaaaaggttttgccaaagcactccttgttgtacgaatattttactgt |
| | | ttacaacgaattgactaaggttaagtacgttaccgaaggtatgagaaagccagctttcttgtctggtgaacaaaagaagg |
| | | ccattgttgatttgttgtttaagaccaacagaaaggttactgtcaagcaattgaaagaagattacttcaagaagatcgaatg |
| | | tttcgattctgtcgagatctccggtgttgaggatagatttaacgcttctttaggtacctaccacgatttattgaagatcatcaa |
| | | ggacaaggatttcttggacaacgaagaaaacgaagatatcttggaagacattgtcttgactttaaccttatttgaagatag |
| | | agaaatgattgaagaaagattgaagacctacgcccacttgttcgatgacaaggtcatgaagcaattgaaaagaagaag |
| | | atacaccggttggggtagattatccagaaagttaattaacggtatcagagataagcaatctggtaagaccatcttggattt |
| | | cttgaagtccgatggtttcgctaacagaaacttcatgcaattgattcatgacgactccttgaccttcaaggaagacattcaa |
| | | aaagctcaagtttccggtcaaggtgactctttgcatgaacacatcgctaacttggccggttccccagctattaagaagggt |
| | | attttgcaaaccgttaaggtcgtcgacgaattagttaaggtcatgggtagacacaagccagaaaacattgttattgaaatg |
| | | agaattgggttcccaaattttaaaggaacacccagttgaaaatactcaattacaaaacgagaagttgtatttgtactatttac |
| | | aaaacggtagagatatgtacgtcgaccaagaattagacatcaaccgtttatctgactacgacgtcgatcacatcgtccca |
| | | caatctttcttgaaggacgattctatcgacaacaaggttttaactcgttctgacaagaatagaggtaaatccgataacgttc |
| | | catccgaagaagtcgtcaaaaagatgaaaaactactggagacaattgttgaacgctaagttgatcacccaaagaaaattt |
| | | gataacttaactaaggctgaacgtggtggtttgtccgaattggacaaggccggtttcattaagagacaattagttgaaacc |
| | | cgtcaaattactaagcacgttgctcaaattttggattcccgtatgaacactaagtacgatgaaaacgacaagttgattaga |
| | | gaggtcaaggttattaccttgaagtccaagttggtttccgacttcagaaaggattttcaattttacaaagttcgtgaaatcaa |
| | | caactatcaccacgctcacgatgcttacttaaacgccgttgtcggtaccgctttgattaaaaagtatccaaagttggaatcc |
| | | gaattcgtctacggtgactacaaggtctacgacgtcagaaaaatgattgctaagtctgaacaagaaattggtaaggctac |
| | | tgctaagtacttcttctattccaacatcatgaacttttttaaaaccgaaatcaccttggctaatggtgaaatcagaaaaagac |
| | | ctttgatcgaaactaacggtgaaaccggtgaaattgtttgggataagggtagagacttcgctaccgttagaaaggttttgt |
| | | ccatgccacaagtcaacattgtcaagaagaccgaagttcaaaccggtggtttctctaaggaatctatcttgccaaagcgt |
| | | aattctgacaaattgattgccagaaagaaggattgggatccaaaaaaatacggtggtttcgattctccaactgttgcttact |
| | | ccgtcttggtcgtcgctaaagtcgaaaagggtaagtctaagaagttgaaatccgtcaaagaattgttgggtatcactattat |
| | | ggaaagatcttctttcgaaaagaacccaatcgatttcttggaagccaagggttacaaggaagttaaaaaggacttgatca |
| | | ttaaattgccaaaatactctttgttcgaattggagaatggtagaaaaagaatgttggcttccgctggtgaattgcaaaagg |
| | | gtaacgaattggctttgccatccaagtacgttaactttttgtacttagcctctcactacgaaaagttgaaaggttccccagaa |
| | | gataacgaacaaaaacaattgttcgtcgaacaacataaacattatttggatgagattattgaacaaatttctgagttttctaa |
| | | gagagtcatcttggccgacgctaacttggataaggtcttgtccgcctacaacaagcacagagacaagccaatcagaga |
| | | acaagccgaaaacatcattcacttgttcactttaactaacttgggtgccccagctgctttcaaatacttcgacactaccattg |
| | | acagaaagagatacacttctactaaggaagtcttggatgctactttgatccaccaatccatcactggtttgtacgaaacta |
| | | gaattgatttgtctcaattgggtggtgatagcagggctgaccccaagaagaagaggaaggtgtag |
| 8 | engineered | agcagggctgaccccaagaagaagaggaaggtg |
| | tag NLS | |
| 9 | terminator | tatataactgtctagaaataaacacccgtcgagcctgtccgatttcaaa |
| | Tsynth11 | |
| 10 | promoter | gtccccgccgggtcacccggccagcgacatggaggcccagaataccctccttgacagtcttgacgtgcgcagctcag |
| | Ag-pTEF | gggcatgatgtgactgtcgcccgtacatttagcccatacatccccatgtataatcatttgcatccatacattttgatggccg |
| | | cacggogcgaagcaaaaattacggctcctcgctgcagacctgcgagcagggaaacgctcccctcacagacgcgttg |
| | | aattgtccccacgccgcgcccctgtagagaaatataaaaggttaggatttgccactgaggttcttctttcatatacttcctttt |
| | | aaaatcttgctaggatacagttctcacatcacatccgaacataaacaacc |
| 11 | Hph gene | atgggtaaaaagcctgaactcaccgcgacgtctgtcgagaagtttctgatcgaaaagttcgacagcgtctccgacctga |
| | | tgcagctctcggagggcgaagaatctcgtgctttcagcttcgatgtaggagggcgtggatatgtcctgcgggtaaatag |
| | | ctgcgccgatggtttctacaaagatcgttatgtttatcggcactttgcatcggccgcgctcccgattccggaagtgcttga |
| | | cattggggaattcagcgagagcctgacctattgcatctcccgccgtgcacagggtgtcacgttgcaagacctgcctgaa |
| | | accgaactgcccgctgttctgcagccggtcgcggaggccatggatgcgatcgctgcggccgatcttagccagacgag |
| | | cgggttcggcccattcggaccgcaaggaatcggtcaatacactacatggcgtgatttcatatgcgcgattgctgatcccc |
| | | atgtgtatcactggcaaactgtgatggacgacaccgtcagtgcgtccgtcgcgcaggctctcgatgagctgatgctttg |
| | | ggccgaggactgccccgaagtccggcacctcgtgcacgcggatttcggctccaacaatgtcctgacggacaatggcc |
| | | gcataacagcggtcattgactggagcgaggcgatgttcggggattcccaatacgaggtcgccaacatcttcttctggag |
| | | gccgtggttggcttgtatggagcagcagacgcgctacttcgagcggaggcatccggagcttgcaggatcgccgcggc |
| | | tccgggcgtatatgctccgcattggtcttgaccaactctatcagagcttggttgacggcaatttcgatgatgcagcttggg |
| | | cgcagggtcgatgcgacgcaatcgtccgatccggagccgggactgtcgggcgtacacaaatcgcccgcagaagcg |
| | | cggccgtctggaccgatggctgtgtagaagtactcgccgatagtggaaaccgacgccccagcactcgtccgagggc |
| | | aaaggaataa |
| 12 | Removal | gctgatccccatgtgtatcactggcaaactgtgatggacgacaccgtcagtgcgtccgtcgcgcaggctctcgatgagc |
| | by | tgatgctttgggccgaggactgccccgaagtccggcacctcgtgcacgcggatttcggctccaacaatgtcctgacgg |
| | Proto- | acaatggccgcataacagcggtcattgactggagcgaggcgatgttcggggattcccaatacgaggtcgccaacatct |
| | trophic | tcttctggaggccgtggttggcttgtatggagcagcagacgcgctacttcgagcggaggcatccggagcttgcaggat |
| | Selection | cgccgcggctccgggcgtatatgctccgcattggtcttgaccaactctatcagagcttggttgacggcaatttcgatgat |
| | (RePS) | gcagcttgggcgcagggtcgatgcgacgcaatcgtccgatccggagccgggactgtcgggcgtacacaaatcgccc |
| | vector | gcagaagcgcggccgtctggaccgatggctgtgtagaagtactcgccgatagtggaaaccgacgccccagcactcg |
| | RePS-B | tccgagggcaaaggaataatcagtactgacaataaaaagattcttgttttcaagaacttgtcatttgtatagtttttttatattgt |
| | sequence | agttgttctattttaatcaaatgttagcgtgatttatattttttttcgcctcgacatcatctgcccagatgcgaagttaagtgcgc |
| | | agaaagtaatatcatgcgtcaatcgtatgtgaatgctggtcgctatactgctgtcgattcgatactaacgccgccatccag |
| | | tgtcgaaaacgagctcgaattcatcgatgaaagacagaaaatttgctgacattggtaatacagtcaaattgcagtactctg |
| | | cgggtgtatacagaatagcagaatgggcagacattacgaatgcacacggtgtggtgggcccaggtattgttagcggttt |
| | | gaagcaggcggcagaagaagtaacaaaggaacctagaggccttttgatgttagcagaattgtcatgcaagggctccct |
| | | atctactggagaatatactaagggtactgttgacattgcgaagagcgacaaagattttgttatcggctttattgctcaaaga |
| | | gacatgggtggaagagatgaaggttacgattggttgattatgacacccggtgtgggtttagatgacaagggagacgcat |
| | | tgggtcaacagtatagaaccgtggatgatgtggtctctacaggatctgacattattattgttggaagaggactatttgcaaa |
| | | gggaagggatgctaaggtagagggtgaacgttacagaaaagcaggctgggaagcatatttgagaagatgcggccag |
| | | caaaactaaaaaactgtattataagtaaatgcatgtatactaaactcacaaattagagcttcaatttaattatatcagttattac |
| | | ccggg |
| 13 | terminator | tcagtactgacaataaaaagattcttgttttcaagaacttgtcatttgtatagtttttttatattgtagttgttctattttaatcaa |
| | KanMX_term | atgttagcgtgatttatattttttttcgcctcgacatcatctgcccagatgcgaagttaagtgcgcagaaagtaatatcatgcgt |
| | | caatcgtatgtgaatgctggtcgctatactgctgtcgattcgatactaacgccgccatccagtgtcgaaaacgagctcga |
| | | attcatcgatga |
| 14 | URA3 | aagacagaaaatttgctgacattggtaatacagtcaaattgcagtactctgcgggtgtatacagaatagcagaatgggca |
| | flanking | gacattacgaatgcacacggtgtggtgggcccaggtattgttagcggtttgaagcaggcggcagaagaagtaacaaa |
| | sequence | ggaacctagaggccttttgatgttagcagaattgtcatgcaagggctccctatctactggagaatatactaagggtactgt |
| | 2 | tgacattgcgaagagcgacaaagattttgttatcggctttattgctcaaagagacatgggtggaagagatgaaggttacg |
| | | attggttgattatgacacccggtgtgggtttagatgacaagggagacgcattgggtcaacagtatagaaccgtggatgat |
| | | gtggtctctacaggatctgacattattattgttggaagaggactatttgcaaagggaagggatgctaaggtagagggtga |
| | | acgttacagaaaagcaggctgggaagcatatttgagaagatgcggccagcaaaactaa |
| 15 | direct | aagacagaaaatttgctgacattggtaatacagtcaaattgcagtactctgcgggtgtatacagaatagcagaatgggca |
| | repeat | gacattacgaatgcacacggtgtggtgggcccaggtattgttagcggtttgaagcaggcggcagaagaagtaacaaa |
| | loop-out | ggaacctagaggccttttgatgttagcagaattgtcatgcaagggctccctatctactggagaatatactaagggtactgt |
| | | tgacattgcgaagagcgacaaagattttgttatcggctttattgctcaaagagacatgggt |
| 16 | terminator | aaaactgtattataagtaaatgcatgtatactaaactcacaaattagagcttcaatttaattatatcagttattacccggg |
| | tURA3 | |
| 17 | pGUIDE- | aacgaagcatctgtgcttcattttgtagaacaaaaatgcaacgcgagagcgctaatttttcaaacaaagaatctgagctgc |
| | 7.1- | atttttacagaacagaaatgcaacgcgaaagcgctattttaccaacgaagaatctgtgcttcatttttgtaaaacaaaaatg |
| | with-MCH5 | caacgcgagagcgctaatttttcaaacaaagaatctgagctgcatttttacagaacagaaatgcaacgcgagagcgcta |
| | | ttttaccaacaaagaatctatacttcttttttgttctacaaaaatgcatcccgagagcgctatttttctaacaaagcatcttagat |
| | | tactttttttctcctttgtgcgctctataatgcagtctcttgataactttttgcactgtaggtccgttaaggttagaagaaggcta |
| | | ctttggtgtctattttctcttccataaaaaaagcctgactccacttcccgcgtttactgattactagcgaagctgcgggtgcat |
| | | tttttcaagataaaggcatccccgattatattctataccgatgtggattgcgcatactttgtgaacagaaagtgatagcgttg |
| | | atgattcttcattggtcagaaaattatgaacggtttcttctattttgtctctatatactacgtataggaaatgtttacattttcgtat |
| | | tgttttcgattcactctatgaatagttcttactacaatttttttgtctaaagagtaatactagagataaacataaaaaatgtagag |
| | | gtcgagtttagatgcaagttcaaggagcgaaaggtggatgggtaggttatatagggatatagcacagagatatatagca |
| | | aagagatacttttgagcaatgtttgtggaagcggtattcgcaatattttagtagctcgttacagtccggtgcgtttttggttttt |
| | | tgaaagtgcgtcttcagagcgcttttggttttcaaaagcgctctgaagttcctatactttctagagaataggaacttcggaat |
| | | aggaacttcaaagcgtttccgaaaacgagcgcttccgaaaatgcaacgcgagctgcgcacatacagctcactgttcac |
| | | gtcgcacctatatctgcgtgttgcctgtatatatatatacatgagaagaacggcatagtgcgtgtttatgcttaaatgcgtac |
| | | ttatatgcgtctatttatgtaggatgaaaggtagtctagtacctcctgtgatattatcccattccatgcggggtatcgtatgctt |
| | | ccttcagcactaccctttagctgttctatatgctgccactcctcaattggattagtctcatccttcaatgctatcatttcctttgat |
| | | attggatcactgggtggaatcccttctgcagcacctggattaccctgttatccctagtacagcccacgttagtccgtcaaat |
| | | tcaggggagatcaccgttgagtcctcatctccctcaagcaggccggccgtagactgccatcgagtctctttgaaaagat |
| | | aatgtatgattatgctttcactcatatttatacagaaacttgatgttttctttcgagtatatacaaggtgattacatgtacgtttga |
| | | agtacaactctagattttgtagtgccctcttgggctagcggtaaaggtgcgcattttttcacaccctacaatgttctgttcaa |
| | | aagattttggtcaaacgctgtagaagtgaaagttggtgcgcatgtttcggcgttcgaaacttctccgcagtgaaagataaa |
| | | tgatcccaaagatggtgagtcacgtgttttagagctagaaatagcaagttaaaataaggctagtccgttatcaacttgaaa |
| | | aagtggcaccgagtcggtggtgctttttttgttttttatgtctcattaccagggaccggagttctggttaattaacaggctcct |
| | | ggtccagagtaccgatctatttgctgatcggtacggtgggctgatcggcgataacctgagctggacggcgacgtaaac |
| | | gogcgcgttaggaacgtacccagtgattctgggtagaagatcggtctgcattggatggtggtaacgcatttttttacacac |
| | | attacttgcctcgagcatcaaatggtggttattcgtggatctatatcacgtgatttgcttaagaattgtcgttcatggtgacac |
| | | ttttagctttgacatgattaagctcatctcaattgatgttatctaaagtcatttcaactatctaagatgtggttgtgattgggcca |
| | | ttttgtgaaagccagtacgccagcgtcaatacactcccgtcaattagttgcaccatgtccacaaaatcatataccagtaga |
| | | gctgagactcatgcaagtccggttgcatcgaaacttttacgtttaatggatgaaaagaagaccaatttgtgtgcttctcttg |
| | | acgttcgttcgactgatgagctattgaaacttgttgaaacgttgggtccatacatttgccttttgaaaacacacgttgatatct |
| | | tggatgatttcagttatgagggtactgtcgttccattgaaagcattggcagagaaatacaagttcttgatatttgaggacag |
| | | aaaattcgccgatatcggtaacacagtcaaattacaatatacatcgggcgtttaccgtatcgcagaatggtctgatatcac |
| | | caacgcccacggggttactggtgctggtattgttgctggcttgaaacaaggtgcgcaagaggtcaccaaagaaccaag |
| | | gggattattgatgcttgctgaattgtcttccaagggttctctagcacacggtgaatatactaagggtaccgttgatattgcaa |
| | | agagtgataaagatttcgttattgggttcattgctcagaacgatatgggaggaagagaagaagggtttgattggctaatca |
| | | tgaccccaggtgtaggtttagacgacaaaggcgatgcattgggtcagcagtacagaaccgtcgacgaagttgtaagtg |
| | | gtggatcagatatcatcattgttggcagaggacttttcgccaagggtagagatcctaaggttgaaggtgaaagatacaga |
| | | aatgctggatgggaagcgtaccaaaagagaatcagcgctccccattaattatacaggaaacttaatagaacaaatcaca |
| | | tatttaatctaatagccacctgcattggcacggtgcaacactacttcaacttcatcttacaaaaagatcacgtgatctgttgt |
| | | attgaactgaaaattttttgtttgcttctctctctctctctttcattatgtgagatttaaaaaccagaaactacatcatcgatgtga |
| | | agagcttcactgagtagggcccgggctgtaaacggtcgatgttccgcggagaggtttgttatccccggcgttaggaact |
| | | acggtggcagatgtagtgtttccacagggcgatcgcctactggtagcaggtagaactatgcggtgtgaaataccgccat |
| | | gaccaaaatcccttaacgtgagttttcgttccactgagcgtcagaccccgtagaaaagatcaaaggatcttcttgagatcc |
| | | tttttttctgcgcgtaatctgctgcttgcaaacaaaaaaaccaccgctaccagcggtggtttgtttgccggatcaagagcta |
| | | ccaactctttttccgaaggtaactggcttcagcagagcgcagataccaaatactgttcttctagtgtagccgtagttaggc |
| | | caccacttcaagaactctgtagcaccgcctacatacctcgctctgctaatcctgttaccagtggctgctgccagtggcgat |
| | | aagtcgtgtcttaccgggttggactcaagacgatagttaccggataaggcgcagcggtcgggctgaacggggggttc |
| | | gtgcacacagcccagcttggagcgaacgacctacaccgaactgagatacctacagcgtgagctatgagaaagcgcc |
| | | acgcttcccgaagggagaaaggcggacaggtatccggtaagcggcagggtcggaacaggagagcgcacgaggg |
| | | agcttccagggggaaacgcctggtatctttatagtcctgtcgggtttcgccacctctgacttgagcgtcgatttttgtgatg |
| | | ctcgtcaggggggcggagcctatggaaaaacgccagcaacgcggcctttttacggttcctggccttttgctggccttttg |
| | | ctcacatgttctttcctgcgttatcccctgattctgtggataaccgtattaccgcctttgagtgagctgataccgctcgccgc |
| | | cgcgcgttggccgattcattaatgcagctggcacgacaggtttcccgactggaaagcgggcagtgagcgcaacgcaa |
| | | ttaatgtgagttagctcactcattaggcaccccaggctttacacggtgagtgagtgtgtgcgtgtggggcgcgccagatg |
| | | ggaacaggatcttgaacttaatcgccttgcagcacatccccctttcgccagctggcgtaatagcgaagaggcccgcac |
| | | cgatcgcccttcccaacagttgcgcagcctgaatggcgaatggcgataagctagcttcacgctgccgcaagcactcag |
| | | ggogcaagggctgctaaaggaagcggaacacgtagaaagccagtccgcagaaacggtgctgaccccggatgaatg |
| | | tcagctactgggctatctggacaagggaaaacgcaagcgcaaagagaaagcaggtagcttgcagtgggcttacatgg |
| | | cgatagctagactgggcggttttatggacagcaagcgaaccggaattgccagctggggcgccctctggtaaggttgg |
| | | gaagccctgcaaagtaaactggatggctttcttgccgccaaggatctgatggcgcaggggatcaagatctgatcaaga |
| | | gacaggatgaggatcgtttcgcatgattgaacaagatggattgcacgcaggttctccggccgcttgggtggagaggct |
| | | attcggctatgactgggcacaacagacaatcggctgctctgatgccgccgtgttccggctgtcagcgcaggggcgccc |
| | | ggttctttttgtcaagaccgacctgtccggtgccctgaatgaactccaagacgaggcagcgcggctatcgtggctggcc |
| | | acgacgggogttccttgcgcagctgtgctcgacgttgtcactgaagcgggaagggactggctgctattgggcgaagtg |
| | | ccggggcaggatctcctgtcatctcaccttgctcctgccgagaaagtatccatcatggctgatgcaatgcggcggctgc |
| | | atacgcttgatccggctacctgcccattcgaccaccaagcgaaacatcgcatcgagcgagcacgtactcggatggaag |
| | | ccggtcttgtcgatcaggatgatctggacgaagagcatcaggggctcgcgccagccgaactgttcgccaggctcaag |
| | | gogoggatgcccgacggcgaggatctcgtcgtgacccatggcgatgcctgcttgccgaatatcatggtggaaaatgg |
| | | ccgcttttctggattcatcgactgtggccggctgggtgtggcggaccgctatcaggacatagcgttggctacccgtgata |
| | | ttgctgaagagcttggcggcgaatgggctgaccgcttcctcgtgctttacggtatcgccgctcccgattcgcagcgcatc |
| | | gccttctatcgccttcttgacgagttcttctgagcgggactctggggttcgctagaggatcgatcctttttaacccatcacat |
| | | atacctgccgttcactattatttagtgaaatgagatattatgatattttctgaattgtgattaaaaaggcaactttatgcccatg |
| | | caacagaaactataaaaaatacagagaatgaaaagaaacagatagattttttagttctttaggcccgtagtctgcaaatcc |
| | | ttttatgattttctatcaaacaaaagaggaaaatagaccagttgcaatccaaacgagagtctaatagaatgaggtcgaaaa |
| | | gtaaatcgcgcgggtttgttactgataaagcaggcaagacctaaaatgtgtaaagggcaaagtgtatactttggcgtcac |
| | | cccttacatattttaggtctttttttattgtgcgtaactaacttgccatcttcaaacaggagggctggaagaagcagaccgct |
| | | aacacagtacataaaaaaggagacatgaacgactccagtctttctagaagatggcaaacagctattatgggtattatggg |
| | | tatttttcaaactgcaaattcaagaaaaagccacgcgtgtgcaccttttttttccccttccagtgcattatgcaatagacagc |
| | | acgagtctttgaaaaagtaacttataaaactgtatcaatttttaaacctaaatagattcataaactattcgttaatataaagtgtt |
| | | ctaaactatgatgaaaaaataagcagaaaagactaataattcttagttaaaagcactttactgatacgtgtccagatcaacc |
| | | gotttcacgacctctaccagacacatgtgatcacggcgctcgtcgcggtctttgctcagtttggtgtggtaggtaatgtgat |
| | | gataacgcgggatatgcactgccgcggagcccgccaacggacgattcatttggctgcatttggtaaccagtttttcggtc |
| | | acaccttcaatatcgtacgcctggttgaactcaacgcggatgccattgttaacggtgtcaggcagaatatacagaatgctt |
| | | ggogggcattggaatgcaacgttcttacgcagaatgtgaccgtctttcttaaagttctcaccagtcagcgtgacacgattg |
| | | tagatagaaccgcgttcgtaggtaaccatagcacgcgtcttgtacacgccgtcgccttcgaagctgatggtacgctcttg |
| | | ggtataaccttccggcatggcgctcttaaagaaatccttgatgtggctcgggtacttggcgaaacactgaacaccgtagc |
| | | tcagggtgctcaccagggttgcccacgggaccggcaggtcgcccgtagtgcagatgtatttcgctttaatggtacccgt |
| | | ggtcgcgtcaccggtaccctcgcctttaatgataaatttcataccttcgacgtcgccttccagttcggtgatatacgggatc |
| | | totttctcaaacagttttgcaccttccgtcaatgccgtcattttgtaattaaaacttagattagattgctatgctttctttctaatga |
| | | gcaagaagtaaaaaaagttgtaatagaacaagaaaaatgaaactgaaacttgagaaattgatgaccgtttattaacttaa |
| | | atatcaatgggaggtcatcgaaagagaaaaaaatcaaaaaaaaaaattttcaagaaaaagaaacgtgataaaaattttta |
| | | ttgcctttttcgacgaagaaaaagaaacgaggcggtgtcttttttcttttccaaacctttagtacgggtaattaacgacaccc |
| | | tagaggaagaaagaggggaaatttagtatgctgtgcttgggtgttttgaagtggtacggcgatgcgcggagtccgaga |
| | | aaatctggaagagtaaaaaaggagtagaaacattttgaagctatggtgtgtgggggatcacttgtgggggattgggtgt |
| | | gatgtaaggattcgcggtcctcgaaaattaaaagtccaacgcgcctgttgcttcctatgtgatatgtattatatgtaatatgc |
| | | ataaatatatctactgcattgtattttgaacgtacaaagtatgcattgtttatacgctattatcagccaaagttgggtggtcgct |
| | | ttctgttgtatgactattgatgtctaggctgtcaataatttcgttttgagcctccatgtctctgaagaactccctgttggcaagg |
| | | aatggcaaactgagcacaacaataccagtcoggatcaactggcaccatctctcccgtagtctcatctaatttttcttccgg |
| | | atgaggttccagatataccgcaacacctttattatggtttccctgagggaataatagaatgtcccattcgaaatcaccaatt |
| | | ctaaacctgggcgaattgtatttcgggtttgttaactcgttccagtcaggaatgttccacgtgaagctatcttccagcaaag |
| | | tctccacttcttcatcaaattgtgggagaatactcccaatgctcttatctatgggacttccgggaaacacagtaccgatactt |
| | | cccaattcgtcttcagagctcattgtttgtttgaagagactaatcaaagaatcgttttctcaaaaaaattaatatcttaactgat |
| | | agtttgatcaaaggggcaaaacgtaggggcaaacaaacggaaaaatcgtttctcaaattttctgatgccaagaactctaa |
| | | ccagtcttatctaaaaattgccttatgatccgtctctccggttacagcctgtgtaactgattaatcctgcctttctaatcaccat |
| | | tctaatgttttaattaagggattttgtcttcattaacggctttcgctcataaaaatgttatgacgttttgcccgcaggcgggaa |
| | | accatccacttcacgagactgatctcctctgccggaacaccgggcatctccaacttataagttggagaaataagagaatt |
| | | tcagattgagagaatgaaaaaaaaaaaaaaaaaaaaggcagaggagagcatagaaatggggttcactttttggtaaag |
| | | ctatagcatgcctatcacatataaatagagtgccagtagcgacttttttcacactcgaaatactcttactactgctctcttgtt |
| | | gtttttatcacttcttgtttcttcttggtaaatagaatatcaagctacaaaaagcatacaatcaactatcaactattaactatatc |
| | | gtaatacaca |
| 18 | cloned | ggtgagtgagtgtgtgcgtgtggggcgcgccagatgggaacaggatcttg |
| | region | |
| | tag 9 | |
| 19 | K1_URA3 | atgtccacaaaatcatataccagtagagctgagactcatgcaagtccggttgcatcgaaacttttacgtttaatggatgaa |
| | coding | aagaagaccaatttgtgtgcttctcttgacgttcgttcgactgatgagctattgaaacttgttgaaacgttgggtccatacatt |
| | sequence | tgccttttgaaaacacacgttgatatcttggatgatttcagttatgagggtactgtcgttccattgaaagcattggcagagaa |
| | | atacaagttcttgatatttgaggacagaaaattcgccgatatcggtaacacagtcaaattacaatatacatcgggcgtttac |
| | | cgtatcgcagaatggtctgatatcaccaacgcccacggggttactggtgctggtattgttgctggcttgaaacaaggtgc |
| | | gcaagaggtcaccaaagaaccaaggggattattgatgcttgctgaattgtcttccaagggttctctagcacacggtgaat |
| | | atactaagggtaccgttgatattgcaaagagtgataaagatttcgttattgggttcattgctcagaacgatatgggaggaa |
| | | gagaagaagggtttgattggctaatcatgaccccaggtgtaggtttagacgacaaaggcgatgcattgggtcagcagta |
| | | cagaaccgtcgacgaagttgtaagtggtggatcagatatcatcattgttggcagaggacttttcgccaagggtagagatc |
| | | ctaaggttgaaggtgaaagatacagaaatgctggatgggaagcgtaccaaaagagaatcagcgctccccattaa |
| 20 | cloned | acagcccacgttagtccgtcaaattcaggggagatcaccg |
| | region | |
| | stuffer | |
| | 4 | |
| 21 | cloned | actccagtctttctagaagatggcaaacagctattatgggtattatgggt |
| | region | |
| | tag 10 | |
| 22 | cloned | attaccagggaccggagttctggttaattaacaggctcctggtccagagt |
| | region | |
| | tag 3 | |
| 23 | DasherGFP | ttactgatacgtgtccagatcaaccgctttcacgacctctaccagacacatgtgatcacggcgctcgtcgcggtctttgct |
| | CDS gene | cagtttggtgtggtaggtaatgtgatgataacgcgggatatgcactgccgcggagcccgccaacggacgattcatttgg |
| | | ctgcatttggtaaccagtttttcggtcacaccttcaatatcgtacgcctggttgaactcaacgcggatgccattgttaacggt |
| | | gtcaggcagaatatacagaatgcttggcgggcattggaatgcaacgttcttacgcagaatgtgaccgtctttcttaaagtt |
| | | ctcaccagtcagcgtgacacgattgtagatagaaccgcgttcgtaggtaaccatagcacgcgtcttgtacacgccgtcg |
| | | ccttcgaagctgatggtacgctcttgggtataaccttccggcatggcgctcttaaagaaatccttgatgtggctcgggtac |
| | | ttggcgaaacactgaacaccgtagctcagggtgctcaccagggttgcccacgggaccggcaggtcgcccgtagtgc |
| | | agatgtatttcgctttaatggtacccgtggtcgcgtcaccggtaccctcgcctttaatgataaatttcataccttcgacgtcg |
| | | ccttccagttcggtgatatacgggatctctttctcaaacagttttgcaccttccgtcaatgccgtcat |
| 24 | cloned | tgtgaagagcttcactgagtagggcccgggctgtaaacggtcgatgttcc |
| | region | |
| | tag 7 | |
| 25 | pSNR 52 | tctttgaaaagataatgtatgattatgctttcactcatatttatacagaaacttgatgttttctttcgagtatatacaaggtgatta |
| | sequence | catgtacgtttgaagtacaactctagattttgtagtgccctcttgggctagcggtaaaggtgcgcattttttcacaccctaca |
| | | atgttctgttcaaaagattttggtcaaacgctgtagaagtgaaagttggtgcgcatgtttcggcgttcgaaacttctccgca |
| | | gtgaaagataaatgatc |
| 26 | sgRNA | gttttagagctagaaatagcaagttaaaataaggctagtccgttatcaacttgaaaaagtggcaccgagtcggtggtgctt |
| | structural | tttttgtttttta |
| | sequence | |
| 27 | origin of | aacgaagcatctgtgcttcattttgtagaacaaaaatgcaacgcgagagcgctaatttttcaaacaaagaatctgagctgc |
| | replica- | atttttacagaacagaaatgcaacgcgaaagcgctattttaccaacgaagaatctgtgcttcatttttgtaaaacaaaaatg |
| | tion 2u ori | caacgcgagagcgctaatttttcaaacaaagaatctgagctgcatttttacagaacagaaatgcaacgcgagagcgcta |
| | sequence | ttttaccaacaaagaatctatacttcttttttgttctacaaaaatgcatcccgagagcgctatttttctaacaaagcatcttagat |
| | | tactttttttctcctttgtgcgctctataatgcagtctcttgataactttttgcactgtaggtccgttaaggttagaagaaggcta |
| | | ctttggtgtctattttctcttccataaaaaaagcctgactccacttcccgcgtttactgattactagcgaagctgcgggtgcat |
| | | tttttcaagataaaggcatccccgattatattctataccgatgtggattgcgcatactttgtgaacagaaagtgatagcgttg |
| | | atgattcttcattggtcagaaaattatgaacggtttcttctattttgtctctatatactacgtataggaaatgtttacattttcgtat |
| | | tgttttcgattcactctatgaatagttcttactacaatttttttgtctaaagagtaatactagagataaacataaaaaatgtagag |
| | | gtcgagtttagatgcaagttcaaggagcgaaaggtggatgggtaggttatatagggatatagcacagagatatatagca |
| | | aagagatacttttgagcaatgtttgtggaagcggtattcgcaatattttagtagctcgttacagtccggtgcgtttttggttttt |
| | | tgaaagtgcgtcttcagagcgcttttggttttcaaaagcgctctgaagttcctatactttctagagaataggaacttcggaat |
| | | aggaacttcaaagcgtttccgaaaacgagcgcttccgaaaatgcaacgcgagctgcgcacatacagctcactgttcac |
| | | gtcgcacctatatctgcgtgttgcctgtatatatatatacatgagaagaacggcatagtgcgtgtttatgcttaaatgcgtac |
| | | ttatatgcgtctatttatgtaggatgaaaggtagtctagtacctcctgtgatattatcccattccatgcggggtatcgtatgctt |
| | | ccttcagcactaccctttagctgttctatatgctgccactcctcaattggattagtctcatccttcaatgctatcatttcctttgat |
| | | attggatc |
| 28 | cloned | ataacctgagctggacggcgacgtaaacgcgcgcgttaggaacgtaccca |
| | region | |
| | tag 4 | |
| 29 | protein | gaagttcctatactttctagagaataggaacttcggaataggaacttc |
| | binding | |
| | site FRT | |
| 30 | cloned | accgatctatttgctgatcggtacggtgggctgatcggcg |
| | region | |
| | stuffer | |
| | 5 | |
| 31 | origin of | ttgagatcctttttttctgcgcgtaatctgctgcttgcaaacaaaaaaaccaccgctaccagcggtggtttgtttgccggatc |
| | replica- | aagagctaccaactctttttccgaaggtaactggcttcagcagagcgcagataccaaatactgttcttctagtgtagccgt |
| | tion MBI | agttaggccaccacttcaagaactctgtagcaccgcctacatacctcgctctgctaatcctgttaccagtggctgctgcca |
| | ORI | gtggcgataagtcgtgtcttaccgggttggactcaagacgatagttaccggataaggcgcagcggtcgggctgaacg |
| | sequence | gggggttcgtgcacacagcccagcttggagcgaacgacctacaccgaactgagatacctacagcgtgagctatgaga |
| | | aagcgccacgcttcccgaagggagaaaggcggacaggtatccggtaagcggcagggtcggaacaggagagcgca |
| | | cgagggagcttccaggggggggggaaacgcctggtatctttatagtcctgtcgggtttcgccacctctgacttgagcgtcgatttt |
| | | tgtgatgctcgtcaggggggcggagcctatggaaaaacgcc |
| 32 | MCH5 | ccaaagatggtgagtcacgt |
| | spacer | |
| 33 | terminator | attatacaggaaacttaatagaacaaatcacatatttaatctaatagccacctgcattggcacggtgcaacactacttcaac |
| | T_URA3 | ttcatcttacaaaaagatcacgtgatctgttgtattgaactgaaaattttttgtttgcttctctctctctctctttcattatgtg |
| | | agatttaaaaaccagaaactacatcatcga |
| 34 | promoter | gtgattctgggtagaagatcggtctgcattggatggtggtaacgcatttttttacacacattacttgcctcgagcatcaaatg |
| | P_URA3 | gtggttattcgtggatctatatcacgtgatttgcttaagaattgtcgttcatggtgacacttttagctttgacatgattaagctc |
| | | atctcaattgatgttatctaaagtcatttcaactatctaagatgtggttgtgattgggccattttgtgaaagccagtacgccag |
| | | cgtcaatacactcccgtcaattagttgcacc |
| 35 | nptII | atgattgaacaagatggattgcacgcaggttctccggccgcttgggtggagaggctattcggctatgactgggcacaac |
| | gene | agacaatcggctgctctgatgccgccgtgttccggctgtcagcgcaggggcgcccggttctttttgtcaagaccgacct |
| | | gtccggtgccctgaatgaactccaagacgaggcagcgcggctatcgtggctggccacgacgggcgttccttgcgcag |
| | | ctgtgctcgacgttgtcactgaagcgggaagggactggctgctattgggcgaagtgccggggcaggatctcctgtcat |
| | | ctcaccttgctcctgccgagaaagtatccatcatggctgatgcaatgcggcggctgcatacgcttgatccggctacctgc |
| | | ctggacgaagagcatcaggggctcgcgccagccgaactgttcgccaggctcaaggcgcggatgcccgacggcgag |
| | | gatctcgtcgtgacccatggcgatgcctgcttgccgaatatcatggtggaaaatggccgcttttctggattcatcgactgt |
| | | ggccggctgggtgtggcggaccgctatcaggacatagcgttggctacccgtgatattgctgaagagcttggcggcga |
| | | atgggctgaccgcttcctcgtgctttacggtatcgccgctcccgattcgcagcgcatcgccttctatcgccttcttgacga |
| | | gttcttctga |
| 36 | cloned | ttgagtcctcatctccctcaagcaggccggccgtagactgccatcgagtc |
| | region | |
| | tag 2 | |
| 37 | cloned | gcagatgtagtgtttccacagggcgatcgcctactggtagcaggtagaac |
| | region | |
| | tag 8 | |
| 38 | promoter | aatggcaaactgagcacaacaataccagtccggatcaactggcaccatctctcccgtagtctcatctaatttttcttccgg |
| | pADH2 | atgaggttccagatataccgcaacacctttattatggtttccctgagggaataatagaatgtcccattcgaaatcaccaatt |
| | | ctaaacctgggcgaattgtatttcgggtttgttaactcgttccagtcaggaatgttccacgtgaagctatcttccagcaaag |
| | | tctccacttcttcatcaaattgtgggagaatactcccaatgctcttatctatgggacttccgggaaacacagtaccgatactt |
| | | cccaattcgtcttcagagctcattgtttgtttgaagagactaatcaaagaatcgttttctcaaaaaaattaatatcttaactgat |
| | | agtttgatcaaaggggcaaaacgtaggggcaaacaaacggaaaaatcgtttctcaaattttctgatgccaagaactctaa |
| | | ccagtcttatctaaaaattgccttatgatccgtctctccggttacagcctgtgtaactgattaatcctgcctttctaatcaccat |
| | | tctaatgttttaattaagggattttgtcttcattaacggctttcgctcataaaaatgttatgacgttttgcccgcaggcgggaa |
| | | accatccacttcacgagactgatctcctctgccggaacaccgggcatctccaacttataagttggagaaataagagaatt |
| | | tcagattgagagaatgaaaaaaaaaaaaaaaaaaaaggcagaggagagcatagaaatggggttcactttttggtaaag |
| | | ctatagcatgcctatcacatataaatagagtgccagtagcgacttttttcacactcgaaatactcttactactgctctcttgtt |
| | | gtttttatcacttcttgtttcttcttggtaaatagaatatcaagctacaaaaagcatacaatcaactatcaactattaactatatc |
| | | gtaatacaca |
| 39 | terminator | atttttcaaactgcaaattcaagaaaaagccacgcgtgtgcaccttttttttccccttccagtgcattatgcaatagacagca |
| | ScENO2 | cgagtctttgaaaaagtaacttataaaactgtatcaatttttaaacctaaatagattcataaactattcgttaatataaagtgttc |
| | | taaactatgatgaaaaaataagcagaaaagactaataattcttagttaaaagcact |
| 40 | stuffer 8 | gcggagaggtttgttatccccggcgttaggaactacggtg |
| 41 | cloned | actgggtggaatcccttctgcagcacctggattaccctgttatccctagt |
| | region | |
| | tag 1 | |
| 42 | promoter | tttgtaattaaaacttagattagattgctatgctttctttctaatgagcaagaagtaaaaaaagttgtaatagaacaagaaaaa |
| | ScTEF 1 | tgaaactgaaacttgagaaattgatgaccgtttattaacttaaatatcaatgggaggtcatcgaaagagaaaaaaatcaaa |
| | | aaaaaaaattttcaagaaaaagaaacgtgataaaaatttttattgcctttttcgacgaagaaaaagaaacgaggcggtgtc |
| | | ttttttcttttccaaacctttagtacgggtaattaacgacaccctagaggaagaaagaggggaaatttagtatgctgtgcttg |
| | | ggtgttttgaagtggtacggcgatgcgcggagtccgagaaaatctggaagagtaaaaaaggagtagaaacattttgaa |
| | | gctatggtgtgtgggggatcacttgtgggggattgggtgtgatgtaaggattcgcggtcctcgaaaattaaaagtccaac |
| | | gcgcctgttgcttcctatgtgatatgtattatatgtaatatgcataaatatatctactgcattgtattttgaacgtacaaagtatg |
| | | cattgtttatacgctattatcagccaaagttgggtggtcgctttctgttgtatgactattgatgtctaggctgtcaataatttcgt |
| | | tttgagcctccatgtctctgaagaactccctgttggcaagg |
| 43 | Tag1- | aatgctatcatttcctttgatattggatc |
| | 2micron | |
| | primer | |
| 44 | RePS- | gtatgtctgttattaatttcacaggtagttctggtccattggtgaaagtttgcggcttgcagagcacagaggccgcagaat |
| | Trp1- | gtgctctagattccgatgctgacttgctgggtattatatgtgtgcccaatagaaagagaacaattgacccggttattgcaa |
| | Kan | ggaaaatttcaagtcttgtaaaagcatataaaaatagttcaggcactccgaaatacttggttggcgtgtttcgtaatcaacc |
| | | taaggaggatgttttggctctggtcaatgattacggcattgatatcgtccaactgcatggagatgagtcgtggcaagaata |
| | | ccaagagttcctcggtttgccagttattaaaagactcgtatttccaaaagactgcaacatactactcagtgcagcttcacag |
| | | aaacctcattcgtttattcccttgtttgattcagaagcaggtgggacaggtgaacttttggattggaactgccaacatttttgt |
| | | ttcttttggacaaatgttgtttgcatttatgatccgttatattttgatctaatgtagagttgcacgtagttcttactggcaaagaa |
| | | aatggcgtggcagaactaactctttatttttccaaatcagaaaaattaatatgttttgccgcgttaaaacctacatcaaaaaa |
| | | ggoggatcaagatgtatgaaagaaagtgcgtagaataacgaacattcatagctgttctggaggctttacaaaaggtaatc |
| | | tttgttaggtgcgattttatccgaaaagcaattactctctatgttagtcatacaaactgacttctgtgagagaattattttcatat |
| | | caacgtaagatcacatggttcctttatcaagtactactatcattccattatatgacctatttacttcttgaatcttagagctcata |
| | | attcaagcaagttgcggagctaagaatttcacatgttgttgaacttaacaatcttcattatacccaatcgctgcgtgctgga |
| | | attatgttaaaagttacatcctttttttcatttttccctacgctcagggcactgtactgcccgtgcctgcgatgagatacatcaa |
| | | tttaaaaaaaaaaccagcatgctataatgctggagcaaaaatttcaatcagaaatagaaaagacctcaacagtaattaac |
| | | ccaaaggggtatcaaataatcgatgtgctttttcactctacgaatgatctgtgagaaactgatttgggccgaatcgcgtaa |
| | | aaagtttgattcgtggcggctaatgtctgaggggctccaacaggctcgtagagcctcgtttcttgagggcacaaaatgtc |
| | | caggtaatattcccaagaaagaaccgcagagtgctttgataaatcggttacaggtcttaacgtaggttttgtctcgctaatt |
| | | gctattgagtaagttcgatccgtttggcgtcttttggggtgtaacgccaaacttattacttttcctatttgaggttggtattgatt |
| | | gttgtcaaagaatgaaaatatacacaaacgccacaatatacgtaccaggttcacgaaaactgatcgtatggttcataccct |
| | | gacttggcaaacctaatgtgaccgtcgctgattagcggatcacgaaaagtgatctcgatacaattagaggatccacgaa |
| | | aatgatgtgaatgaatacatgaaagattcatgagatctgacaacatggtagacgtgtgtgtctcatggaaattgatgcagt |
| | | tgaagacatgtgcgtcacgaaaaaagaaatcaatcctacacagggcttaagggcaaatgtattcatgtgtgtcacgaaa |
| | | agtgatgtaactaaatacacgattaccatggaaattaacgtaccttttttgtgcgtgtattgaaatattatgacatattacaga |
| | | aagggttcgcaagtcctgtttctatgcctttctcttagtaattcacgaaataaacctatggtttacgaaatgatccacgaaaa |
| | | tcatgttattatttacatcaacatatcgcgaaaattcatgtcatgtccacattaacatcattgcagagcaacaattcattttcat |
| | | agagaaatttgctactatcacccactagtactaccattggtacctactactttgaattgtactaccgctgggcgttattaggt |
| | | gtgaaaccacgaaaagttcaccataacttcgaataaagtcgcggaaaaaagtaaacagctattgctactcaaatgaggtt |
| | | tgcagaagcttgttgaagcatgatgaagcgttctaaacgcactattcatcattaaatatttaaagctcataaaattgtattcaa |
| | | ttcctattctaaatggcttttatttctattacaactattagctctaaatccatatcctcataagcagcaatcaattctatctatactt |
| | | cgttatgtgcgcagatggctccgctgcccgcgtcataaatgtcacacagggctatcagaaaatctataatatacagcaaa |
| | | aaaccaaacacagagcttttgaaggtgaacctggtaggttagatcccaggcgtagaacagtttatcagcgtcttgcatta |
| | | caatgtactgcaggtcataaattgtcagtcagggtccctaccaaaccactgttggaaaaaagtggtagaaatgccacca |
| | | aatataaagtgagatggagaaatctgcagcaatgtcagacgcttgatggtaggataataataattccaaaaaaccatcat |
| | | aagacattcccaatgacagttgaaggtgagtttgccgcaaaacgcttcatagaagaaatggagcgctctaaaggagaat |
| | | atttcaactttgacattgaagttagagatttggattatcttgatgctcaattgagaatttctagctgcataagatttggtccagt |
| | | actcacaggaaatggtgttttatctaaatttctcactggacgtagtgaccttgtaactcctgctgtaaaaagtatggcttgga |
| | | tgcttggtctgtggttaggtgacggtacaacaaaagagccagaaatctcagtagatagcttggatcctaagctaatggag |
| | | agtttaagagaaaatgcgaaaatctggggtctctaccttacggtttgtgacgatcacgttccgctacgtgccaaacatgta |
| | | aggcttcattatggagatggtccagatgaaaacaggaagacaaggaatttgaggaaaaataatccattctggaaagctg |
| | | tcacaattttaaagtttaaaagggatcttgatggagagaagcaaatccctgaatttatgtacggcgagcatatagaagttc |
| | | gtgaagcattcttagccggcttgatcgactcagatgggtacgttgtgaaaaagggcgaaggccctgaatcttataaaata |
| | | gcaattcaaactgtttattcatccattatggacggaattgtccatatttcaagatctcttggtatgtcagctactgtgacgacc |
| | | aggtcagctagggaggaaatcattgaaggaagaaaagtccaatgtcaatttacatacgactgtaatgttgctgggggaa |
| | | caactttacagaatgttttgtcatattgtcgaagtggtcacaaaacaagagaagttccgccaattataaaaagggaacccg |
| | | tatatttcagcttcacggatgatttccagggtgagagtactgtatatgggcttacgatagaaggccataaaaatttcttgctt |
| | | ggcaacaaaatagaagtgaaatcatgtcgaggctgctgtgtgggagaacagcataaaatatcacaaaaaaagaatcta |
| | | aaacactgtgttgcttgtcccagaaagggaatcaagtatttttataaagattggagtggtaaaaatcgagtatgtgctagat |
| | | gctatggaagatacaaattcagcggtcatcactgtataaattgcaagtatgtaccagaagcacgtgaagtgaaaaaggc |
| | | aaaagacaaaggcgaaaaattgggcattacgcccgaaggtttgccagttaaaggaccagagtgtataaaatgtggcg |
| | | gaatcttacagtttgatgctgtccgcgggcctcataagagttgtggtaacaacgcaggtgcgcgcatctgctaaaatgtg |
| | | tatattagtttaaaaagttgtatgtaataaaagtaaaatttaatattttggatgaaaaaaaccatttttagactttttcttaactag |
| | | aatgctggagtagaaatacgccatctcaagatacaaaaagcgttaccggcactgatttgtttcaaccagtatatagattatt |
| | | attgggtcttgatcaactttcctcagacatatcagtaacagttatcaagctaaatatttacgcgaaagaaaaacaaatatttt |
| | | aattgtgatacttgtgaattttattttattaaggatacaaagttaagagaaaacaaaagtccccgccgggtcacccggcca |
| | | gogacatggaggcccagaataccctccttgacagtcttgacgtgcgcagctcaggggcatgatgtgactgtcgcccgt |
| | | acatttagcccatacatccccatgtataatcatttgcatccatacattttgatggccgcacggcgcgaagcaaaaattacg |
| | | gctcctcgctgcagacctgcgagcagggaaacgctcccctcacagacgcgttgaattgtccccacgccgcgcccctg |
| | | tagagaaatataaaaggttaggatttgccactgaggttcttctttcatatacttccttttaaaatcttgctaggatacagttctc |
| | | acatcacatccgaacataaacaaccatgggtaaggaaaagactcacgtttcgaggccgcgattaaattccaacatggat |
| | | gctgatttatatgggtataaatgggctcgcgataatgtcgggcaatcaggtgcgacaatctatcgattgtatgggaagcc |
| | | cgatgcgccagagttgtttctgaaacatggcaaaggtagcgttgccaatgatgttacagatgagatggtcagactaaact |
| | | ggctgacggaatttatgcctcttccgaccatcaagcattttatccgtactcctgatgatgcatggttactcaccactgcgat |
| | | ccccggcaaaacagcattccaggtattagaagaatatcctgattcaggtgaaaatattgttgatgcgctggcagtgttcct |
| | | gcgccggttgcattcgattcctgtttgtaattgtccttttaacagcgatcgcgtatttcgtctcgctcaggcgcaatcacgaa |
| | | tgaataacggtttggttgatgcgagtgattttgatgacgagcgtaatggctggcctgttgaacaagtctggaaagaaatg |
| | | cataagcttttgccattctcaccggattcagtcgtcactcatggtgatttctcacttgataaccttatttttgacgaggggaaa |
| | | ttaataggttgtattgatgttggacgagtcggaatcgcagaccgataccaggatcttgccatcctatggaactgcctcggt |
| | | gagttttctccttcattacagaaacggctttttcaaaaatatggtattgataatcctgatatgaataaattgcagtttcatttgat |
| | | gctcgatgagtttttctaatcagtactgacaataaaaagattcttgttttcaagaacttgtcatttgtatagtttttttatattgtag |
| | | ttgttctattttaatcaaatgttagcgtgatttatattttttttcgcctcgacatcatctgcccagatgcgaagttaagtgcgcag |
| | | aaagtaatatcatgcgtcaatcgtatgtgaatgctggtcgctatactgctgtcgattcgatactaacgccgccatccagtgt |
| | | cgaaaacgagctcgaattcatcgatgattgtaaaagcatataaaaatagttcaggcactccgaaatacttggttggcgtgt |
| | | ttcgtaatcaacctaaggaggatgttttggctctggtcaatgattacggcattgatatcgtccaactgcatggagatgagtc |
| | | gtggcaagaataccaagagttcctcggtttgccagttattaaaagactcgtatttccaaaagactgcaacatactactcag |
| | | tgcagcttcacagaaacctcattcgtttattcccttgtttgattcagaagcaggtgggacaggtgaacttttggattggaact |
| | | cgatttctgactgggttggaaggcaagagagccccgaaagcttacattttatgttagctggtggactgacgccagaaaat |
| | | gttggtgatgcgcttagattaaatggcgttattggtgttgatgtaagcggaggtgtggagacaaatggtgtaaaagactct |
| | | aacaaaatagcaaatttcgtcaaaaatgctaagaaatag |
| 45 | TRP1 | atgtctgttattaatttcacaggtagttctggtccattggtgaaagtttgcggcttgcagagcacagaggccgcagaatgt |
| | flanking | gctctagattccgatgctgacttgctgggtattatatgtgtgcccaatagaaagagaacaattgacccggttattgcaagg |
| | sequence | |
| | 1 | |
| 46 | Removal | atgtctgttattaatttcacaggtagttctggtccattggtgaaagtttgcggcttgcagagcacagaggccgcagaatgt |
| | by | gctctagattccgatgctgacttgctgggtattatatgtgtgcccaatagaaagagaacaattgacccggttattgcaagg |
| | Proto- | aaaatttcaagtcttgtaaaagcatataaaaatagttcaggcactccgaaatacttggttggcgtgtttcgtaatcaacctaa |
| | trophic | ggaggatgttttggctctggtcaatgattacggcattgatatcgtccaactgcatggagatgagtcgtggcaagaatacc |
| | Selection | aagagttcctcggtttgccagttattaaaagactcgtatttccaaaagactgcaacatactactcagtgcagcttcacagaa |
| | (RePS) | acctcattcgtttattcccttgtttgattcagaagcaggtgggacaggtgaacttttggattggaactgccaacatttttgtttc |
| | vector | ttttggacaaatgttgtttgcatttatgatccgttatattttgatctaatgtagagttgcacgtagttcttactggcaaagaaatc |
| | RePS-A | gatgcataccaaaaaagaataaaggtgatatttgatctttaccgtttagttccaacgtaaaattgtgcctttggacttaaaat |
| | sequence | ggcgtggcagaactaactctttatttttccaaatcagaaaaattaatatgttttgccgcgttaaaacctacatcaaaaaagg |
| | | cggatcaagatgtatgaaagaaagtgcgtagaataacgaacattcatagctgttctggaggctttacaaaaggtaatcttt |
| | | gttaggtgcgattttatccgaaaagcaattactctctatgttagtcatacaaactgacttctgtgagagaattattttcatatca |
| | | acgtaagatcacatggttcctttatcaagtactactatcattccattatatgacctatttacttcttgaatcttagagctcataatt |
| | | caagcaagttgcggagctaagaatttcacatgttgttgaacttaacaatcttcattatacccaatcgctgcgtgctggaatt |
| | | atgttaaaagttacatcctttttttcatttttccctacgctcagggcactgtactgcccgtgcctgcgatgagatacatcaattt |
| | | aaaaaaaaaaccagcatgctataatgctggagcaaaaatttcaatcagaaatagaaaagacctcaacagtaattaaccc |
| | | aaaggggtatcaaataatcgatgtgctttttcactctacgaatgatctgtgagaaactgatttgggccgaatcgcgtaaaa |
| | | agtttgattcgtggcggctaatgtctgaggggctccaacaggctcgtagagcctcgtttcttgagggcacaaaatgtcca |
| | | ggtaatattcccaagaaagaaccgcagagtgctttgataaatcggttacaggtcttaacgtaggttttgtctcgctaattgc |
| | | tattgagtaagttcgatccgtttggcgtcttttggggtgtaacgccaaacttattacttttcctatttgaggttggtattgattgtt |
| | | gtcaaagaatgaaaatatacacaaacgccacaatatacgtaccaggttcacgaaaactgatcgtatggttcataccctga |
| | | cttggcaaacctaatgtgaccgtcgctgattagcggatcacgaaaagtgatctcgatacaattagaggatccacgaaaat |
| | | gatgtgaatgaatacatgaaagattcatgagatctgacaacatggtagacgtgtgtgtctcatggaaattgatgcagttga |
| | | agacatgtgcgtcacgaaaaaagaaatcaatcctacacagggcttaagggcaaatgtattcatgtgtgtcacgaaaagt |
| | | gatgtaactaaatacacgattaccatggaaattaacgtaccttttttgtgcgtgtattgaaatattatgacatattacagaaag |
| | | ggttcgcaagtcctgtttctatgcctttctcttagtaattcacgaaataaacctatggtttacgaaatgatccacgaaaatcat |
| | | gttattatttacatcaacatatcgcgaaaattcatgtcatgtccacattaacatcattgcagagcaacaattcattttcataga |
| | | gaaatttgctactatcacccactagtactaccattggtacctactactttgaattgtactaccgctgggcgttattaggtgtga |
| | | aaccacgaaaagttcaccataacttcgaataaagtcgcggaaaaaagtaaacagctattgctactcaaatgaggtttgca |
| | | gaagcttgttgaagcatgatgaagcgttctaaacgcactattcatcattaaatatttaaagctcataaaattgtattcaattcc |
| | | tattctaaatggcttttatttctattacaactattagctctaaatccatatcctcataagcagcaatcaattctatctatactttaaa |
| | | atgctttctgaaaacacgactattctgatggctaacggtgaaattaaagacatcgcaaacgtcacggctaactcttacgtt |
| | | atgtgcgcagatggctccgctgcccgcgtcataaatgtcacacagggctatcagaaaatctataatatacagcaaaaaa |
| | | ccaaacacagagcttttgaaggtgaacctggtaggttagatcccaggcgtagaacagtttatcagcgtcttgcattacaat |
| | | gtactgcaggtcataaattgtcagtcagggtccctaccaaaccactgttggaaaaaagtggtagaaatgccaccaaatat |
| | | aaagtgagatggagaaatctgcagcaatgtcagacgcttgatggtaggataataataattccaaaaaaccatcataaga |
| | | cattcccaatgacagttgaaggtgagtttgccgcaaaacgcttcatagaagaaatggagcgctctaaaggagaatatttc |
| | | aactttgacattgaagttagagatttggattatcttgatgctcaattgagaatttctagctgcataagatttggtccagtactca |
| | | caggaaatggtgttttatctaaatttctcactggacgtagtgaccttgtaactcctgctgtaaaaagtatggcttggatgctt |
| | | ggtctgtggttaggtgacggtacaacaaaagagccagaaatctcagtagatagcttggatcctaagctaatggagagttt |
| | | aagagaaaatgcgaaaatctggggtctctaccttacggtttgtgacgatcacgttccgctacgtgccaaacatgtaaggc |
| | | ttcattatggagatggtccagatgaaaacaggaagacaaggaatttgaggaaaaataatccattctggaaagctgtcac |
| | | aattttaaagtttaaaagggatcttgatggagagaagcaaatccctgaatttatgtacggcgagcatatagaagttcgtga |
| | | agcattcttagccggcttgatcgactcagatgggtacgttgtgaaaaagggcgaaggccctgaatcttataaaatagcaa |
| | | ttcaaactgtttattcatccattatggacggaattgtccatatttcaagatctcttggtatgtcagctactgtgacgaccaggt |
| | | cagctagggaggaaatcattgaaggaagaaaagtccaatgtcaatttacatacgactgtaatgttgctgggggaacaac |
| | | tttacagaatgttttgtcatattgtcgaagtggtcacaaaacaagagaagttccgccaattataaaaagggaacccgtatat |
| | | ttcagcttcacggatgatttccagggtgagagtactgtatatgggcttacgatagaaggccataaaaatttcttgcttggca |
| | | acaaaatagaagtgaaatcatgtcgaggctgctgtgtgggagaacagcataaaatatcacaaaaaaagaatctaaaac |
| | | actgtgttgcttgtcccagaaagggaatcaagtatttttataaagattggagtggtaaaaatcgagtatgtgctagatgctat |
| | | ggaagatacaaattcagcggtcatcactgtataaattgcaagtatgtaccagaagcacgtgaagtgaaaaaggcaaaa |
| | | gacaaaggogaaaaattgggcattacgcccgaaggtttgccagttaaaggaccagagtgtataaaatgtggcggaatc |
| | | ttacagtttgatgctgtccgcgggcctcataagagttgtggtaacaacgcaggtgcgcgcatctgctaaaatgtgtatatt |
| | | aaaatttcaagtcttgtaaaagcatataaaaatagttcaggcactccgaaatacttggttggcgtgtttcgtaatcaacctaa |
| | | ggaggatgttttggctctggtcaatgattacggcattgatatcgtccaactgcatggagatgagtcgtggcaagaatacc |
| | | aagagttcctcggtttgccagttattaaaagactcgtatttccaaaagactgcaacatactactcagtgcagcttcacagaa |
| | | acctcattcgtttattcccttgtttgattcagaagcaggtgggacaggtgaacttttggattggaact |
| | | agtttaaaaagttgtatgtaataaaagtaaaatttaatattttggatgaaaaaaaccatttttagactttttcttaactagaatgc |
| | | tggagtagaaatacgccatctcaagatacaaaaagcgttaccggcactgatttgtttcaaccagtatatagattattattgg |
| | | gtcttgatcaactttcctcagacatatcagtaacagttatcaagctaaatatttacgcgaaagaaaaacaaatattttaattgt |
| | | gatacttgtgaattttattttattaaggatacaaagttaagagaaaacaaaagtccccgccgggtcacccggccagcgac |
| | | atggaggcccagaataccctccttgacagtcttgacgtgcgcagctcaggggcatgatgtgactgtcgcccgtacattt |
| | | agcccatacatccccatgtataatcatttgcatccatacattttgatggccgcacggcgcgaagcaaaaattacggctcct |
| | | cgctgcagacctgcgagcagggaaacgctcccctcacagacgcgttgaattgtccccacgccgcgcccctgtagaga |
| | | aatataaaaggttaggatttgccactgaggttcttctttcatatacttccttttaaaatcttgctaggatacagttctcacatcac |
| | | atccgaacataaacaaccatgggtaaggaaaagactcacgtttcgaggccgcgattaaattccaacatggatgctgattt |
| | | atatgggtataaatgggctcgcgataatgtcgggcaatcaggtgcgacaatctatcgattgtatgggaagcccgatgcg |
| | | ccagagttgtttctgaaacatggcaaaggtagcgttgccaatgatgttacagatgagatggtcagactaaactggctgac |
| | | ggaatttatgcctcttccgaccatcaagcattttatccgtactcctgatgatgcatggttactcaccactgcgatccccggc |
| | | aaaacagcattccaggtattagaagaatatcctgattcaggtgaaaatattgttgatgcgctggcagtgttcctgcgccgg |
| | | ttgcattcgattcctgtttgtaattgtccttttaacagcgatcgcgtatttcgtctcgctcaggcgcaatcacgaatgaataac |
| | | ggtttggttgatgcgagtgattttgatgacgagcgtaatggctggc |
| 47 | direct | ttgtaaaagcatataaaaatagttcaggcactccgaaatacttggttggcgtgtttcgtaatcaacctaaggaggatgtttt |
| | repeat | ggctctggtcaatgattacggcattgatatcgtccaactgcatggagatgagtcgtggcaagaataccaagagttcctcg |
| | loop out | gtttgccagttattaaaagactcgtatttccaaaagactgcaacatactactcagtgcagcttcacagaaacctcattcgttt |
| | | attcccttgtttgattcagaagcaggtgggacaggtgaacttttggattggaact |
| 48 | HO | gccaacatttttgtttcttttggacaaatgttgtttgcatttatgatccgttatattttgatctaatgtagagttgcacgtagttctt |
| | promoter | actggcaaagaaatcgatgcataccaaaaaagaataaaggtgatatttgatctttaccgtttagttccaacgtaaaattgtg |
| | | cctttggacttaaaatggcgtggcagaactaactctttatttttccaaatcagaaaaattaatatgttttgccgcgttaaaacc |
| | | tacatcaaaaaaggoggatcaagatgtatgaaagaaagtgcgtagaataacgaacattcatagctgttctggaggcttta |
| | | caaaaggtaatctttgttaggtgcgattttatccgaaaagcaattactctctatgttagtcatacaaactgacttctgtgagag |
| | | aattattttcatatcaacgtaagatcacatggttcctttatcaagtactactatcattccattatatgacctatttacttcttgaatc |
| | | ttagagctcataattcaagcaagttgcggagctaagaatttcacatgttgttgaacttaacaatcttcattatacccaatcgct |
| | | gcgtgctggaattatgttaaaagttacatcctttttttcatttttccctacgctcagggcactgtactgcccgtgcctgcgatg |
| | | agatacatcaatttaaaaaaaaaaccagcatgctataatgctggagcaaaaatttcaatcagaaatagaaaagacctcaa |
| | | cagtaattaacccaaaggggtatcaaataatcgatgtgctttttcactctacgaatgatctgtgagaaactgatttgggccg |
| | | aatcgcgtaaaaagtttgattcgtggcggctaatgtctgaggggctccaacaggctcgtagagcctcgtttcttgagggc |
| | | acaaaatgtccaggtaatattcccaagaaagaaccgcagagtgctttgataaatcggttacaggtcttaacgtaggttttg |
| | | tctcgctaattgctattgagtaagttcgatccgtttggcgtcttttggggtgtaacgccaaacttattacttttcctatttgaggt |
| | | tggtattgattgttgtcaaagaatgaaaatatacacaaacgccacaatatacgtaccaggttcacgaaaactgatcgtatg |
| | | gttcataccctgacttggcaaacctaatgtgaccgtcgctgattagcggatcacgaaaagtgatctcgatacaattagag |
| | | gatccacgaaaatgatgtgaatgaatacatgaaagattcatgagatctgacaacatggtagacgtgtgtgtctcatggaa |
| | | attgatgcagttgaagacatgtgcgtcacgaaaaaagaaatcaatcctacacagggcttaagggcaaatgtattcatgtg |
| | | tgtcacgaaaagtgatgtaactaaatacacgattaccatggaaattaacgtaccttttttgtgcgtgtattgaaatattatgac |
| | | atattacagaaagggttcgcaagtcctgtttctatgcctttctcttagtaattcacgaaataaacctatggtttacgaaatgat |
| | | ccacgaaaatcatgttattatttacatcaacatatcgogaaaattcatgtcatgtccacattaacatcattgcagagcaacaa |
| | | ttcattttcatagagaaatttgctactatcacccactagtactaccattggtacctactactttgaattgtactaccgctgggc |
| | | gttattaggtgtgaaaccacgaaaagttcaccataacttcgaataaagtcgcggaaaaaagtaaacagctattgctactc |
| | | aaatgaggtttgcagaagcttgttgaagcatgatgaagcgttctaaacgcactattcatcattaaatatttaaagctcataaa |
| | | attgtattcaattcctattctaaatggcttttatttctattacaactattagctctaaatccatatcctcataagcagcaatcaatt |
| | | ctatctatacttt |
| 49 | HO endo- | gccaacatttttgtttcttttggacaaatgttgtttgcatttatgatccgttatattttgatctaatgtagagttgcacgtagttctt |
| | nuclease | actggcaaagaaatcgatgcataccaaaaaagaataaaggtgatatttgatctttaccgtttagttccaacgtaaaattgtg |
| | tran- | cctttggacttaaaatggcgtggcagaactaactctttatttttccaaatcagaaaaattaatatgttttgccgcgttaaaacc |
| | scription | tacatcaaaaaaggoggatcaagatgtatgaaagaaagtgcgtagaataacgaacattcatagctgttctggaggcttta |
| | unit | caaaaggtaatctttgttaggtgcgattttatccgaaaagcaattactctctatgttagtcatacaaactgacttctgtgagag |
| | | aattattttcatatcaacgtaagatcacatggttcctttatcaagtactactatcattccattatatgacctatttacttcttgaatc |
| | | ttagagctcataattcaagcaagttgcggagctaagaatttcacatgttgttgaacttaacaatcttcattatacccaatcgct |
| | | gcgtgctggaattatgttaaaagttacatcctttttttcatttttccctacgctcagggcactgtactgcccgtgcctgcgatg |
| | | agatacatcaatttaaaaaaaaaaccagcatgctataatgctggagcaaaaatttcaatcagaaatagaaaagacctcaa |
| | | cagtaattaacccaaaggggtatcaaataatcgatgtgctttttcactctacgaatgatctgtgagaaactgatttgggccg |
| | | aatcgcgtaaaaagtttgattcgtggcggctaatgtctgaggggctccaacaggctcgtagagcctcgtttcttgagggc |
| | | acaaaatgtccaggtaatattcccaagaaagaaccgcagagtgctttgataaatcggttacaggtcttaacgtaggttttg |
| | | tctcgctaattgctattgagtaagttcgatccgtttggcgtcttttggggtgtaacgccaaacttattacttttcctatttgaggt |
| | | tggtattgattgttgtcaaagaatgaaaatatacacaaacgccacaatatacgtaccaggttcacgaaaactgatcgtatg |
| | | gttcataccctgacttggcaaacctaatgtgaccgtcgctgattagcggatcacgaaaagtgatctcgatacaattagag |
| | | gatccacgaaaatgatgtgaatgaatacatgaaagattcatgagatctgacaacatggtagacgtgtgtgtctcatggaa |
| | | attgatgcagttgaagacatgtgcgtcacgaaaaaagaaatcaatcctacacagggcttaagggcaaatgtattcatgtg |
| | | tgtcacgaaaagtgatgtaactaaatacacgattaccatggaaattaacgtaccttttttgtgcgtgtattgaaatattatgac |
| | | atattacagaaagggttcgcaagtcctgtttctatgcctttctcttagtaattcacgaaataaacctatggtttacgaaatgat |
| | | ccacgaaaatcatgttattatttacatcaacatatcgcgaaaattcatgtcatgtccacattaacatcattgcagagcaacaa |
| | | ttcattttcatagagaaatttgctactatcacccactagtactaccattggtacctactactttgaattgtactaccgctgggc |
| | | gttattaggtgtgaaaccacgaaaagttcaccataacttcgaataaagtcgcggaaaaaagtaaacagctattgctactc |
| | | aaatgaggtttgcagaagcttgttgaagcatgatgaagcgttctaaacgcactattcatcattaaatatttaaagctcataaa |
| | | attgtattcaattcctattctaaatggcttttatttctattacaactattagctctaaatccatatcctcataagcagcaatcaatt |
| | | ctatctatactttaaaatgctttctgaaaacacgactattctgatggctaacggtgaaattaaagacatcgcaaacgtcacg |
| | | gctaactcttacgttatgtgcgcagatggctccgctgcccgcgtcataaatgtcacacagggctatcagaaaatctataat |
| | | atacagcaaaaaaccaaacacagagcttttgaaggtgaacctggtaggttagatcccaggcgtagaacagtttatcagc |
| | | gtcttgcattacaatgtactgcaggtcataaattgtcagtcagggtccctaccaaaccactgttggaaaaaagtggtagaa |
| | | atgccaccaaatataaagtgagatggagaaatctgcagcaatgtcagacgcttgatggtaggataataataattccaaaa |
| | | aaccatcataagacattcccaatgacagttgaaggtgagtttgccgcaaaacgcttcatagaagaaatggagcgctcta |
| | | aaggagaatatttcaactttgacattgaagttagagatttggattatcttgatgctcaattgagaatttctagctgcataagatt |
| | | tggtccagtactcacaggaaatggtgttttatctaaatttctcactggacgtagtgaccttgtaactcctgctgtaaaaagta |
| | | tggcttggatgcttggtctgtggttaggtgacggtacaacaaaagagccagaaatctcagtagatagcttggatcctaag |
| | | ctaatggagagtttaagagaaaatgcgaaaatctggggtctctaccttacggtttgtgacgatcacgttccgctacgtgcc |
| | | aaacatgtaaggcttcattatggagatggtccagatgaaaacaggaagacaaggaatttgaggaaaaataatccattct |
| | | ggaaagctgtcacaattttaaagtttaaaagggatcttgatggagagaagcaaatccctgaatttatgtacggcgagcat |
| | | atagaagttcgtgaagcattcttagccggcttgatcgactcagatgggtacgttgtgaaaaagggcgaaggccctgaat |
| | | cttataaaatagcaattcaaactgtttattcatccattatggacggaattgtccatatttcaagatctcttggtatgtcagctact |
| | | gtgacgaccaggtcagctagggaggaaatcattgaaggaagaaaagtccaatgtcaatttacatacgactgtaatgttg |
| | | ctgggggaacaactttacagaatgttttgtcatattgtcgaagtggtcacaaaacaagagaagttccgccaattataaaaa |
| | | gggaacccgtatatttcagcttcacggatgatttccagggtgagagtactgtatatgggcttacgatagaaggccataaa |
| | | aatttcttgcttggcaacaaaatagaagtgaaatcatgtcgaggctgctgtgtgggagaacagcataaaatatcacaaaa |
| | | aaagaatctaaaacactgtgttgcttgtcccagaaagggaatcaagtatttttataaagattggagtggtaaaaatcgagt |
| | | atgtgctagatgctatggaagatacaaattcagcggtcatcactgtataaattgcaagtatgtaccagaagcacgtgaagt |
| | | gaaaaaggcaaaagacaaaggcgaaaaattgggcattacgcccgaaggtttgccagttaaaggaccagagtgtataa |
| | | aatgtggcggaatcttacagtttgatgctgtccgcgggcctcataagagttgtggtaacaacgcaggtgcgcgcatctg |
| | | ctaaaatgtgtatattagtttaaaaagttgtatgtaataaaagtaaaatttaatattttggatgaaaaaaaccatttttagactttt |
| | | tcttaactagaatgctggagtagaaatacgccatctcaagatacaaaaagcgttaccggcactgatttgtttcaaccagta |
| | | tatagattattattgggtcttgatcaactttcctcagacatatcagtaacagttatcaagctaaatatttacgcgaaagaaaaa |
| | | caaatattttaattgtgatacttgtgaattttattttattaaggatacaaagttaagagaaaacaaaa |
| 50 | regulatory | tgttaaaagttacatcctttttttcatttttccctacgctcagggcactgtactgcccgtgcctgcgatgagatacatcaattta |
| | sequence | aaaaaaaaaccagcatgctataatgctggagcaaaaatttcaatcagaaatagaaaagacctcaacagtaattaaccca |
| | URS1 | aaggggtatcaaataatcgatgtgctttttcactctacgaatgatctgtgagaaactgatttgggccgaatcgcgtaaaaa |
| | | gtttgattcgtggcggctaatgtctgaggggctccaacaggctcgtagagcctcgtttcttgagggcacaaaatgtccag |
| | | gtaatattcccaagaaagaaccgcagagtgctttgataaatcggttacaggtcttaacgtaggttttgtctcgct |
| 51 | HO endo- | atgctttctgaaaacacgactattctgatggctaacggtgaaattaaagacatcgcaaacgtcacggctaactcttacgtt |
| | nuclease | atgtgcgcagatggctccgctgcccgcgtcataaatgtcacacagggctatcagaaaatctataatatacagcaaaaaa |
| | gene | ccaaacacagagcttttgaaggtgaacctggtaggttagatcccaggcgtagaacagtttatcagcgtcttgcattacaat |
| | | gtactgcaggtcataaattgtcagtcagggtccctaccaaaccactgttggaaaaaagtggtagaaatgccaccaaatat |
| | | aaagtgagatggagaaatctgcagcaatgtcagacgcttgatggtaggataataataattccaaaaaaccatcataaga |
| | | cattcccaatgacagttgaaggtgagtttgccgcaaaacgcttcatagaagaaatggagcgctctaaaggagaatatttc |
| | | aactttgacattgaagttagagatttggattatcttgatgctcaattgagaatttctagctgcataagatttggtccagtactca |
| | | caggaaatggtgttttatctaaatttctcactggacgtagtgaccttgtaactcctgctgtaaaaagtatggcttggatgctt |
| | | ggtctgtggttaggtgacggtacaacaaaagagccagaaatctcagtagatagcttggatcctaagctaatggagagttt |
| | | aagagaaaatgcgaaaatctggggtctctaccttacggtttgtgacgatcacgttccgctacgtgccaaacatgtaaggc |
| | | ttcattatggagatggtccagatgaaaacaggaagacaaggaatttgaggaaaaataatccattctggaaagctgtcac |
| | | aattttaaagtttaaaagggatcttgatggagagaagcaaatccctgaatttatgtacggcgagcatatagaagttcgtga |
| | | agcattcttagccggcttgatcgactcagatgggtacgttgtgaaaaagggcgaaggccctgaatcttataaaatagcaa |
| | | ttcaaactgtttattcatccattatggacggaattgtccatatttcaagatctcttggtatgtcagctactgtgacgaccaggt |
| | | cagctagggaggaaatcattgaaggaagaaaagtccaatgtcaatttacatacgactgtaatgttgctgggggaacaac |
| | | tttacagaatgttttgtcatattgtcgaagtggtcacaaaacaagagaagttccgccaattataaaaagggaacccgtatat |
| | | ttcagcttcacggatgatttccagggtgagagtactgtatatgggcttacgatagaaggccataaaaatttcttgcttggca |
| | | acaaaatagaagtgaaatcatgtcgaggctgctgtgtgggagaacagcataaaatatcacaaaaaaagaatctaaaac |
| | | actgtgttgcttgtcccagaaagggaatcaagtatttttataaagattggagtggtaaaaatcgagtatgtgctagatgctat |
| | | ggaagatacaaattcagcggtcatcactgtataaattgcaagtatgtaccagaagcacgtgaagtgaaaaaggcaaaa |
| | | gacaaaggogaaaaattgggcattacgcccgaaggtttgccagttaaaggaccagagtgtataaaatgtggcggaatc |
| | | ttacagtttgatgctgtccgcgggcctcataagagttgtggtaacaacgcaggtgcgcgcatctgctaa |
| 52 | HO-del-L | aatgtgtatattagtttaaaaagttgtatgtaataaaagtaaaatttaat |
| | sequence | |
| 53 | HO/YDL228C | aatgtgtatattagtttaaaaagttgtatgtaataaaagtaaaatttaatattttggatgaaaaaaaccatttttagactttttctta |
| | intergenic | actagaatgctggagtagaaatacgccatctcaagatacaaaaagcgttaccggcactgatttgtttcaaccagtatatag |
| | terminator | attattattgggtcttgatcaactttcctcagacatatcagtaacagttatcaagctaaatatttacgcgaaagaaaaacaaa |
| | | tattttaattgtgatacttgtgaattttattttattaaggatacaaagttaagagaaaacaaaa |
| 54 | Auto- | taaaagtaaaatttaatattttggatgaaaaaaaccatttttagactttttct |
| | nomously | |
| | Repli- | |
| | cating | |
| | Sequence | |
| | ARS404 | |
| 55 | Ag-pTEF | gtccccgccgggtcacccggccagcgacatggaggcccagaataccctccttgacagtcttgacgtgcgcagctcag |
| | promoter | gggcatgatgtgactgtcgcccgtacatttagcccatacatccccatgtataatcatttgcatccatacattttgatggccg |
| | | cacggcgcgaagcaaaaattacggctcctcgctgcagacctgcgagcagggaaacgctcccctcacagacgcgttg |
| | | aattgtccccacgccgcgcccctgtagagaaatataaaaggttaggatttgccactgaggttcttctttcatatacttcctttt |
| | | aaaatcttgctaggatacagttctcacatcacatccgaacataaacaacc |
| 56 | KanMX6_ | atgggtaaggaaaagactcacgtttcgaggccgcgattaaattccaacatggatgctgatttatatgggtataaatgggct |
| | G418_ | cgcgataatgtcgggcaatcaggtgcgacaatctatcgattgtatgggaagcccgatgcgccagagttgtttctgaaac |
| | resistance | atggcaaaggtagcgttgccaatgatgttacagatgagatggtcagactaaactggctgacggaatttatgcctcttccg |
| | gene | accatcaagcattttatccgtactcctgatgatgcatggttactcaccactgcgatccccggcaaaacagcattccaggta |
| | | ttagaagaatatcctgattcaggtgaaaatattgttgatgcgctggcagtgttcctgcgccggttgcattcgattcctgtttgt |
| | | aattgtccttttaacagcgatcgcgtatttcgtctcgctcaggcgcaatcacgaatgaataacggtttggttgatgcgagtg |
| | | attttgatgacgagcgtaatggctggcctgttgaacaagtctggaaagaaatgcataagcttttgccattctcaccggattc |
| | | agtcgtcactcatggtgatttctcacttgataaccttatttttgacgaggggaaattaataggttgtattgatgttggacgagt |
| | | cggaatcgcagaccgataccaggatcttgccatcctatggaactgcctcggtgagttttctccttcattacagaaacggct |
| | | ttttcaaaaatatggtattgataatcctgatatgaataaattgcagtttcatttgatgctcgatgagtttttctaa |
| 57 | Removal | aacagcattccaggtattagaagaatatcctgattcaggtgaaaatattgttgatgcgctggcagtgttcctgcgccggtt |
| | by | gcattcgattcctgtttgtaattgtccttttaacagcgatcgcgtatttcgtctcgctcaggcgcaatcacgaatgaataacg |
| | Proto- | gtttggttgatgcgagtgattttgatgacgagcgtaatggctggcctgttgaacaagtctggaaagaaatgcataagctttt |
| | trophic | gccattctcaccggattcagtcgtcactcatggtgatttctcacttgataaccttatttttgacgaggggaaattaataggttg |
| | Selection | tattgatgttggacgagtcggaatcgcagaccgataccaggatcttgccatcctatggaactgcctcggtgagttttctcc |
| | (RePS) | ttcattacagaaacggctttttcaaaaatatggtattgataatcctgatatgaataaattgcagtttcatttgatgctcgatgag |
| | vector | tttttctaatcagtactgacaataaaaagattcttgttttcaagaacttgtcatttgtatagtttttttatattgtagttgttctat |
| | RePS-B | tttaatcaaatgttagcgtgatttatattttttttcgcctcgacatcatctgcccagatgcgaagttaagtgcgcagaaagtaatat |
| | sequence | catgcgtcaatcgtatgtgaatgctggtcgctatactgctgtcgattcgatactaacgccgccatccagtgtcgaaaacga |
| | | gctcgaattcatcgatgattgtaaaagcatataaaaatagttcaggcactccgaaatacttggttggcgtgtttcgtaatca |
| | | acctaaggaggatgttttggctctggtcaatgattacggcattgatatcgtccaactgcatggagatgagtcgtggcaag |
| | | aataccaagagttcctcggtttgccagttattaaaagactcgtatttccaaaagactgcaacatactactcagtgcagcttc |
| | | acagaaacctcattcgtttattcccttgtttgattcagaagcaggtgggacaggtgaacttttggattggaactcgatttctg |
| | | actgggttggaaggcaagagagccccgaaagcttacattttatgttagctggtggactgacgccagaaaatgttggtga |
| | | tgcgcttagattaaatggogttattggtgttgatgtaagcggaggtgtggagacaaatggtgtaaaagactctaacaaaat |
| | | agcaaatttcgtcaaaaatgctaagaaatag |
| 58 | marker | aacagcattccaggtattagaagaatatcctgattcaggtgaaaatattgttgatgcgctggcagtgttcctgcgccggtt |
| | overlap | gcattcgattcctgtttgtaattgtccttttaacagcgatcgcgtatttcgtctcgctcaggcgcaatcacgaatgaataacg |
| | sequence | gtttggttgatgcgagtgattttgatgacgagcgtaatggctggc |
| 59 | KanMX | tcagtactgacaataaaaagattcttgttttcaagaacttgtcatttgtatagtttttttatattgtagttgttctattttaatca |
| | terminator | aatgttagcgtgatttatattttttttcgcctcgacatcatctgcccagatgcgaagttaagtgcgcagaaagtaatatcatgcgt |
| | | caatcgtatgtgaatgctggtcgctatactgctgtcgattcgatactaacgccgccatccagtgtcgaaaacgagctcga |
| | | attcatcgatga |
| 60 | TRP1 | ttgtaaaagcatataaaaatagttcaggcactccgaaatacttggttggcgtgtttcgtaatcaacctaaggaggatgtttt |
| | flanking | ggctctggtcaatgattacggcattgatatcgtccaactgcatggagatgagtcgtggcaagaataccaagagttcctcg |
| | sequence | gtttgccagttattaaaagactcgtatttccaaaagactgcaacatactactcagtgcagcttcacagaaacctcattcgttt |
| | 2 | attcccttgtttgattcagaagcaggtgggacaggtgaacttttggattggaactcgatttctgactgggttggaaggcaa |
| | | gagagccccgaaagcttacattttatgttagctggtggactgacgccagaaaatgttggtgatgcgcttagattaaatgg |
| | | cgttattggtgttgatgtaagcggaggtgtggagacaaatggtgtaaaagactctaacaaaatagcaaatttcgtcaaaa |
| | | atgctaagaaatag |
| 61 | direct | ttgtaaaagcatataaaaatagttcaggcactccgaaatacttggttggcgtgtttcgtaatcaacctaaggaggatgtttt |
| | repeat | ggctctggtcaatgattacggcattgatatcgtccaactgcatggagatgagtcgtggcaagaataccaagagttcctcg |
| | loop | gtttgccagttattaaaagactcgtatttccaaaagactgcaacatactactcagtgcagcttcacagaaacctcattcgttt |
| | out | attcccttgtttgattcagaagcaggtgggacaggtgaacttttggattggaact |

The present description is made with reference to the accompanying drawings and Examples, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they disclose essential matter.

### EXAMPLES

### Example 1: Exemplary Prototrophic Gene Editing Method

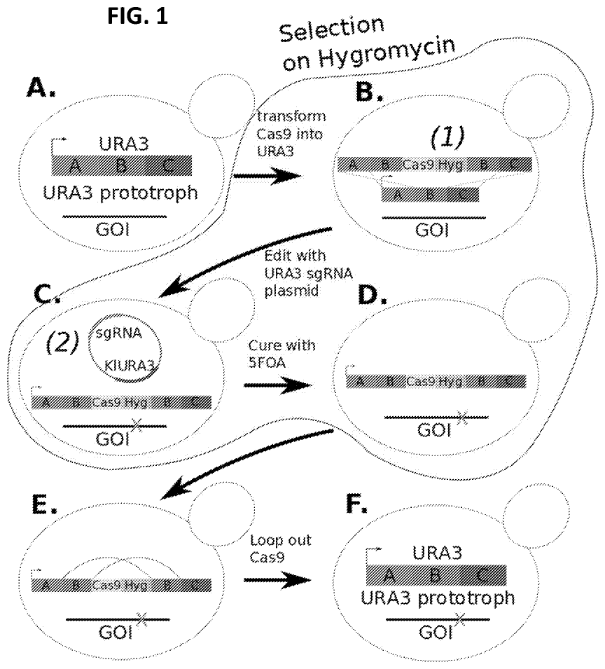

The present disclosure provides methods for isolating a strain of a microorganism with a desired genetic edit (e.g., a mutation to a gene of interest), with no other residual nucleic acids left over from the gene editing process, e.g., DNA expressing the gRNA or Cas nuclease. The present example provides general details of an illustrative method of the disclosure as applied to the model organism Saccharomyces cerevisiae. FIG. 1 shows exemplary components of the illustrative method as applied to genome editing in yeast. The method begins with a haploid, heterothallic yeast strain prototrophic for uracil and containing a wild-type allele of the URA3 gene (FIG. 1A). In the first step of the method, the URA3 gene is disrupted with a Removal by Prototrophic Selection (RePS) vector (1) (FIG. 1B), which contains an expression cassette for a Cas nuclease (such as Cas9, e.g. SEQ ID NO: 7) and a dominant selectable marker (such as HygR, which is selectable by hygromycin, e.g., SEQ ID NO: 11) flanked by repeat sequences that when recombined restore a wild-type allele of URA3 (e.g., SEQ ID NOS: 4 and 14) (FIG. 1B). See, e.g., the exemplary RePS vector of SEQ ID NO: 1. This is accomplished by transforming yeast with DNA construct (1) and selecting for hygromycin-resistant cells. The strain is now a uracil auxotroph and is resistant to hygromycin. Hygromycin selection is maintained to select against the loop-out of Cas9. To accomplish genome editing, DNA construct (2) (e.g., SEQ ID NO: 17) is introduced into the cell along with a repair fragment that introduces an edit to the gene of interest (GOI) (FIG. 1C). The plasmid encodes a homolog of the ScURA3 gene, such as the URA3 gene from Kluyveromyces lactis (K1URA3) (e.g., SEQ ID NO: 19), that complements the uracil prototrophy but is not able to recombine with the S. cerevisiae genome. Yeast transformed with the plasmid are selected for with media lacking uracil that contains hygromycin. The Cas9/sgRNA RNP then causes a dsDNA break which is repaired by the repair fragment and selects against cells that retain the wild-type sequence since these cells are susceptible to dsDNA breaks caused by the RNP. Once the edit is made, the plasmid is removed with selection on media containing 5-FOA, which selects against the K1URA3 gene (FIG. 1D). The Cas nuclease is removed from the genome by selection on media lacking uracil (FIG. 1E). This selects for cells with recombination between the repeats flanking the Cas9-HygR cassette. The final strain is a prototroph with the gene of interest edited, but without other extraneous nucleic acid changes leftover from the gene editing process (FIG. 1F).

### Example 2: Prototrophic Gene Editing of Exemplary Yeast Strain

The general method laid out in Example 1 was applied to exemplary yeast strain CEN.PK 113-7D.

### Introduction of RePS Vector Expressing Cas9

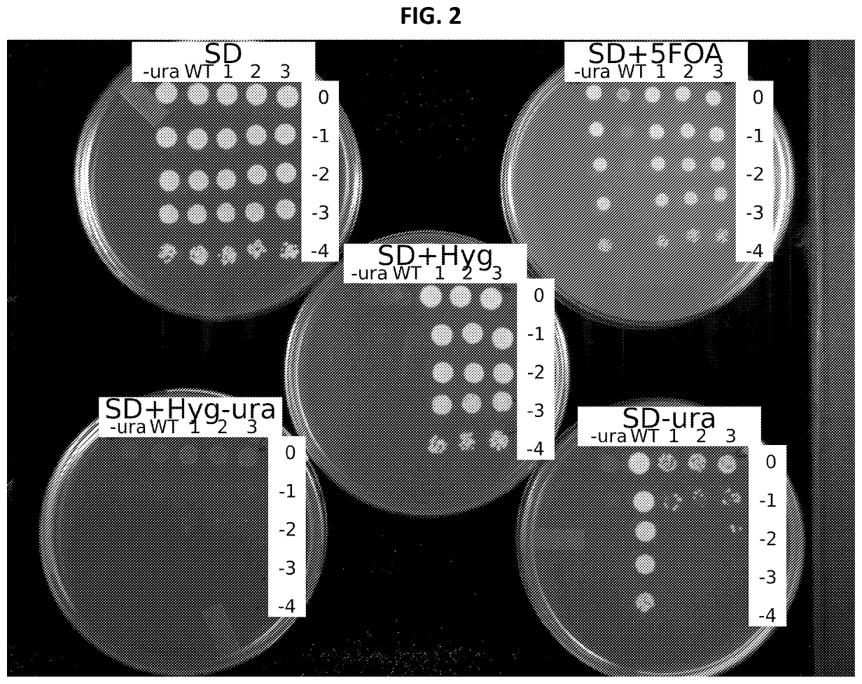

First, assays were conducted to determine whether yeast with an exemplary integrating nucleic acid construct, a RePS vector (SEQ ID NO: 1) containing Cas9 and a HygR cassette and with URA3 repeat regions (1), when used to disrupt the URA3 gene in the genome of yeast would grow on selective and counter-selective media in a way that was consistent with requirements for plasmid selection, counterselection, and Cas9 loop-out. A RePS vector containing a Cas9 nuclease (SEQ ID NO: 7) and hygromycin selectable marker (SEQ ID NO: 11) with flanking URA3 repeat regions (SEQ ID NOS: 4 and 14) was used to disrupt the URA3 gene of the haploid heterothallic yeast strain CEN.PK 113-7D. Using spot-plating, the yeast were tested to determine whether integration of the vector disrupted the function of URA3 and whether the Cas9-HygR coding region could be removed by selection on media lacking uracil (FIG. 2). Plates were spotted with 7.5 μL of 1:10 dilution series of overnight culture of WT, −ura (WT with endogenous URA3 knocked out), and 3 integrants of the Cas9-HygR cassette. Each of the integrants were confirmed to have the cassette at the URA3 locus by amplifying flanks with PCR.

For these integrants, it was predicted that selection on hygromycin containing media would prevent recombination of the repeats RePS vector containing the Cas9-HygR cassette, resulting in the maintenance of an auxotrophic strain. Consistent with these expectations, the strains containing Cas9 integrated at the URA3 locus were not able to grow on media lacking uracil in the presence of hygromycin, and there were no colonies that would be consistent with Cas9 loop-out. Furthermore, strains with Cas9-HygR integrated at URA3 were able to grow on media containing 5-FOA, demonstrating that URA3 was disrupted and that 5-FOA counter-selection could be applied to remove a plasmid from this strain, as needed. When cells were plated on media lacking uracil in the absence of hygromycin, a small number of URA⁺ colonies were isolated, consistent with recombination between the repeats flanking Cas9-HygR. Sanger sequencing confirmed that four of these colonies had a wild-type sequence at the URA3 locus. As a negative control, it was observed that, in the absence of uracil and the presence of hygromycin, none of the strains could grow.

Taken together, these results suggest that integrated Cas9 at the URA3 locus could enable the workflow shown in FIG. 1, and more generally, that the introduction of an integrating nucleic acid construct, e.g., a RePS vector, to a prototrophic gene can enable a method of the present disclosure, such as the one described in Example 1.

### Introduction of Non-Integrating Nucleic Acid Construct

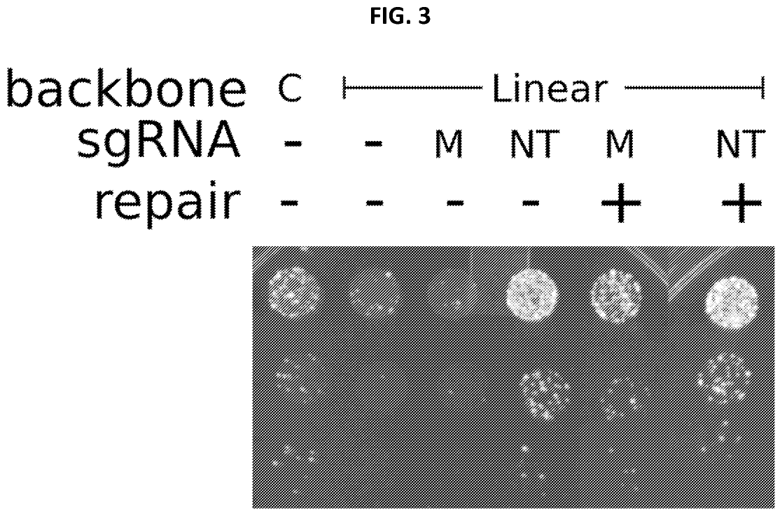

Next, exemplary yeast cells were tested to determine whether Cas9 integrated at URA3 using a RePS vector supports genome editing. To test whether the Cas9-HygR cassette would enable genome editing with a guide RNA expressed from a plasmid, a yeast strain that had the Cas9-HygR cassette integrated in its genome was transformed with different combinations of DNA sequences encoding: (a) a 2μ ORI, URA3 selectable marker, and GFP gene (the “backbone”); (b) a cassette expressing an sgRNA targeting the MCH5 gene with homology to the backbone such that homologous recombination would produce a circularized plasmid capable of replication in yeast (SEQ ID NO: 17); and (c) repair fragments that when incorporated in the yeast genome remove the protospacer targeted by the sgRNA (FIG. 3). Plates comprising SD+Hyg-ura media were spotted with 7.5 μL of 1:10 dilution series of cultures of yeast with different combinations of circularized backbone, linear backbone, sgRNA fragments, and repair fragments. Circularized plasmid, formed by homologous recombination of the backbone and sgRNA cassette, was selected for because the media lacked uracil, which selected for URA3-comprising cells, but the media contained hygromycin to maintain the Cas9-HygR cassette at the endogenous URA3 locus. When both a linear backbone and an sgRNA cassette targeting the genome were supplied, a large reduction in CFU was observed relative to controls where only a control circular backbone was transformed or when the linear backbone and a non-targeting sgRNA cassette was supplied (FIG. 3), consistent with killing by the Cas9 RNP. This loss of CFU was rescued by supplying a repair template, suggesting that the repair template was incorporated into the genome.

To test this hypothesis, PCR was performed with primers specific to the deletion of the wild-type protospacer to genotype the colonies isolated from the transformations. Table 5 shows the results of the structural PCR that was performed. Genotyping results are shown for colonies picked from the transformations in FIG. 3. Immediately after picking, colonies were genotyped with PCR primers that yielded different sized bands depending on whether the cells were edited at the MCH5 locus: MCH5 (original) vs Δmch5 (edited). From the transformation that included both a targeting sgRNA and the repair template, nine out of ten of the genotyped colonies were edited, as shown in Table 5.

TABLE 5
Structural PCR Results for editing gene of interest

| | MCH5 | Δmch5 |
|---|---|---|
| sgRNA + repair | 1 | 9 |
| NT sgRNA + repair | 9 | 1 |
| Circular plasmid | 10 | 0 |

### Removal of Non-Integrating Nucleic Acid Construct and CAS9-HygR Cassette

Next, it was verified that the plasmid expressing the sgRNA could be removed by 5-FOA counterselection and then that CAS9-HygR cassette loop-out could be selected for by growth on media lacking uracil.

The backbone of the plasmid expressing the sgRNAs contained a cassette expressing GFP (SEQ ID NO: 23). This enabled the use of fluorescence to distinguish between URA⁺ colonies resulting from Cas9 loop-out and URA⁺ colonies resulting from cells containing the plasmid. Colonies were picked from the transformations shown in FIG. 3, grown overnight in non-selective media, and then diluted into media containing both 5-FOA and hygromycin: 5-FOA selected against plasmid-containing cells, and hygromycin selected for Cas9 cassette-containing cells. At this point, it was expected that the plasmid would be lost from the cells. To complete the workflow, cells were plated on media lacking uracil, which would select for Cas9 loop-out and the restoration of endogenous URA3 function.

The resulting colonies were examined with blue light to see whether they were white, which would indicate that the plasmid had been lost and Cas9 had looped out, or green, which would indicate that the plasmid had been retained. Although some green colonies were observed, white colonies were readily identified. These white colonies were picked and genotyped with PCR primers designed to test for Cas9 loop-out. The primers yielded different sized bands depending on whether the cells retained the CAS9-HygR cassette at the URA3 locus: ura3Δ::CAS9-HygR (retained cassette) vs. URA3 (URA3 restored). Out of 25 colonies tested, 23 were observed to produce a PCR product that corresponded to Cas9 loop-out, as shown in Table 6.

TABLE 6
Structural PCR Results for CAS9-HygR loop out

| | ura3Δ::CAS9-HygR | URA3 |
|---|---|---|
| sgRNA + repair | 0 | 10 |
| NT sgRNA + repair | 2 | 8 |
| Circular plasmid | 0 | 5 |

Taken together, these results indicate that the exemplary method successfully enabled (a) the editing of a genome and (b) the simple removal of all CRISPR related DNA.

### Results

These data demonstrate that a RePS vector expressing Cas9 (1) (SEQ ID NO: 1) can be introduced into the genome of yeast, can be maintained by antibiotic selection, can support genome editing, and can be removed by recombination restoring endogenous URA3 function. Furthermore, the data demonstrate that an exemplary non-integrating nucleic acid construct (2) (SEQ ID NO: 17) targeting the genome of the yeast can be introduced using uracil selection and removed using 5-FOA counterselection.

### Example 3: Illustrative Implementation of RePS Vectors for Genome Engineering

The present example provides an exemplary implementation of Removal by Prototrophic Selection (RePS) vectors for genome engineering resulting in strains comprising the desired gene edits without extraneous genetic alterations from the gene editing process.

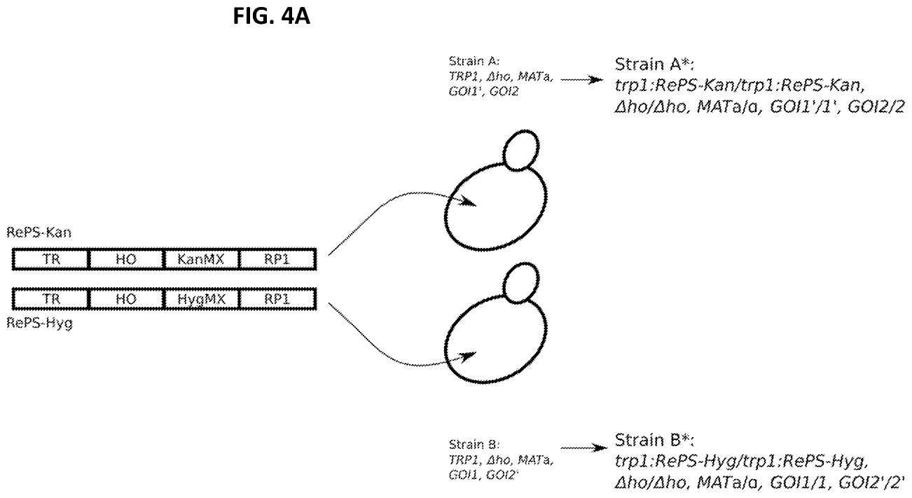

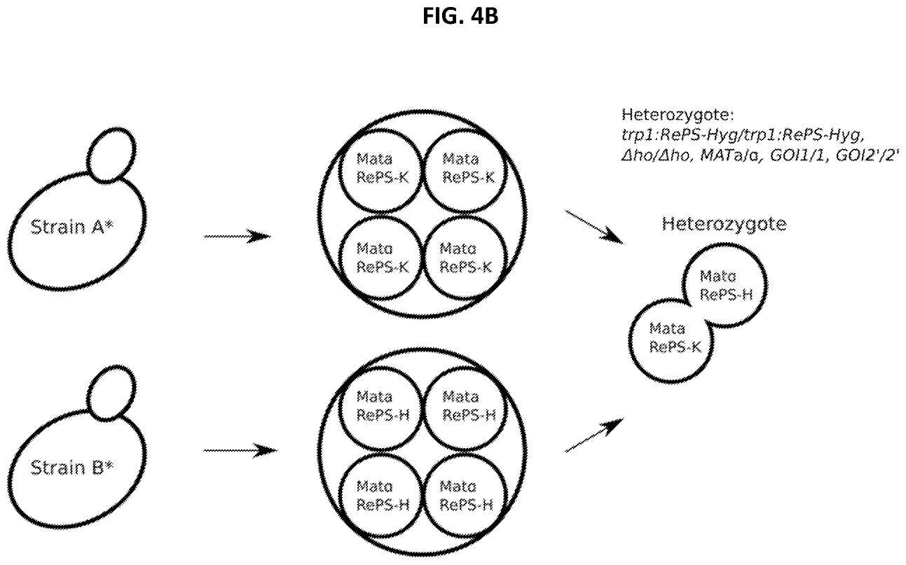

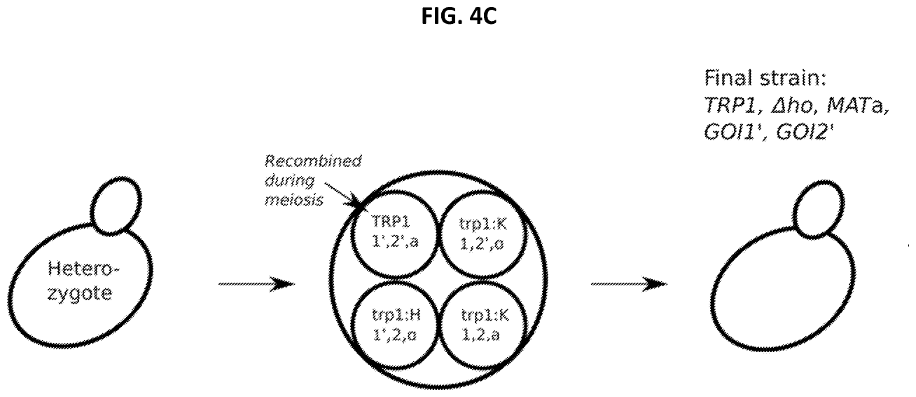

In the present example, RePS vectors are used to generate yeast strains comprising edits to two genes of interest: gene of interest 1 (GOI1) and gene of interest 2 (GOI2). The edited versions of the genes are called GOI1′ and GOI2′. The diagrams in FIG. 4A-4C provide an overview of how the RePS vectors are used to generate a haploid strain containing two edits from two haploid strains containing either.

Step 1: Transform Haploid Starting Strains with RePS Vectors

In Step 1 (FIG. 4A), the RePS vectors are transformed into the TRP1 locus of two haploid yeast strains (Strain A and Strain B). Strain A comprises a desired genetic edit to GOI1 (GOI1′) and Strain B comprises a desired genetic edit to G012 (GOI2′).

The RePS vectors (e.g., SEQ ID NO: 44) comprise the HO gene (e.g., SEQ ID NO: 51) and a dominant selectable marker (antibiotic resistance gene KanMX, e.g., SEQ ID NO: 56, or HygR, e.g., SEQ ID NO: 11) flanked by TRP1 repeats that when recombined can restore the function of TRP1 (e.g., SEQ ID NOS: 45 and 60). The HO nuclease is introduced under the control of the native promoter and terminator to the cell in order to allow mating between strains with different edits. The native promoter ensures the HO expression is limited to the appropriate phase of the cell cycle, which prevents undesirable exogenous double-stranded DNA breaks.

The vectors are integrated into the host genome by selecting for the respective antibiotic resistance located between the repeats of the RePS vector with the antibiotic geneticin (G418) or hygromycin. The integration of these vectors disrupts the function of TRP1, creating tryptophan auxotrophs, such that tryptophan must be supplemented in the growth media until step 3. Antibiotic selection is also maintained until step 3 in order to select against recombination between the repeat regions.

Since haploids are transformed with the HO gene, the first daughter produced by transformed cells mating-type switches and mates with the mother cell to form a diploid. These homozygous diploid strains are referred to as Strain A* and Strain B*.

Step 2: Sporulate, Random Mating, Selection for Heterozygotes with Double Antibiotic Resistance

In Step 2 (FIG. 4B), haploids are generated by meiosis through sporulating Strain A* and Strain B*. These haploids are then allowed to mate with each other. The diploid formed by mating between haploids of Strain A* and Strain B* is selected for by double selection for the antibiotic markers in the RePS vectors (geneticin and hygromycin in this case).

### Step 3: Sporulate, Select for Prototrophs Formed During Meiosis, Screen for Genotype of Interest

In Step 3 (FIG. 4C), a second round of meiosis is used to generate haploids from the heterozygote. Antibiotic selection is relaxed during this step. The haploids will have a mixture of mating types and genotypes at GOI1 and G012. During meiosis, some haploids will have a recombination event between the repeats in the RePS vectors that restores the prototrophy of that haploid. Asci are disrupted and spores are spread on media that selects for tryptophan prototrophs. Only haploids that are prototrophic for tryptophan will germinate and proliferate. Haploid segregants are then screened for the desired combination of genotypes—in this case GOI1′/GOI2′.

### Results

At the end of the process detailed above, haploid strains are recovered with two edited genes but with otherwise the same genotype as the starting haploids. Strains resulting from this process are ready for high-throughput screening and subsequent cycles of genomic engineering.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world.

### Numbered Embodiments of the Disclosure

Notwithstanding the claims provided herein, the following embodiments are contemplated according to the present disclosure.
1. A method for producing a population of gene-edited cells free of gene-editing system molecules, comprising:
   (a) introducing an integrating nucleic acid construct into a population of cells that comprise a target gene of interest and that are prototrophic for a nutrient,
      wherein the integrating nucleic acid construct integrates into a gene that is required for prototrophy for the nutrient; and
      wherein the integrating nucleic acid construct comprises:
         a first nucleotide sequence encoding a gene-editing protein;
         a second nucleotide sequence encoding a dominant selectable marker; and
         a pair of repeat nucleotide sequences flanking the first nucleotide sequence and the second nucleotide sequence;
   (b) selecting for expression of the dominant selectable marker to produce a population of cells that are auxotrophic for the nutrient;
   (c) introducing a non-integrating nucleic acid construct into the population of cells produced in step (b);
      wherein the non-integrating nucleic acid construct comprises:
         a third nucleotide sequence encoding a gene-editing nucleic acid that introduces an edit into the gene of interest; and
         a fourth nucleotide sequence encoding a protein that complements the auxotrophy for the nutrient, wherein the fourth nucleotide sequence cannot recombine with the cellular genome;
   (d) simultaneously selecting for expression of the dominant selectable marker and for prototrophy for the nutrient to produce a population of cells that comprise the edited gene of interest;
   (e) removing the non-integrating nucleic acid nucleic acid construct from the population of cells produced in step (d) by growing the cells on media that selects against expression of the protein that complements the auxotrophy for the nutrient to produce a population of cells that comprise the edited gene of interest and are free of the non-integrating nucleic acid construct; and
   (f) removing the integrating nucleic acid construct from the population of cells produced in step (e) by growing the cells on media that selects for prototrophy for the nutrient to produce a population of cells that comprise the edited gene of interest and that are free of the integrating nucleic acid construct.
2. The method of embodiment 1, wherein the cells are fungal cells or bacterial cells.
3. The method of any one of embodiments 1-2, wherein the fungal cells are Fusarium spp., Kluyveromyces spp., Penicillium spp., Pichia spp., Saccharomyces spp., Schizosaccharomyces spp. or Yarrowia spp.
4. The method of any one of embodiments 1-3, wherein the fungal cells are Kluyveromyces lactis, Kluyveromyces marxianus, Pichia pastoris, Saccharomyces cerevisiae, Schizosaccharomyces pombe or Yarrowia lipolytica.
5. The method of any one of embodiments 1-4, wherein the bacterial cells are Agrobacterium spp., Arthrobacterspecies spp., Bacillus spp., Clostridium spp., Corynebacterium spp., Cupriavidus spp., Escherichia spp., Erwinia spp., Geobacillus spp., Lactobacillus spp., Pantoea spp., Propionibacterium spp., Pseudomonas spp., Sphingomonas spp., Streptococcus spp., Streptomyces spp., Xanthomonas spp., or Zymomonas spp.
6. The method of any one of embodiments 1-5, wherein the bacterial cells are Bacillus clausii, Bacillus lichenifonnis, Bacillus subtilis, Clostridium acetobutylicum, Corynebacterium glutamicum, Cupriavidus necator, Escherichia coli, Geobacillus thermoglucosidasius, Propionibacterium freudenreichii, Sphingomonas elodea, or Xanthomonas campestris.
7. The method of any one of embodiments 1-6, wherein the gene-editing protein is an endonuclease.
8. The method of any one of embodiments 1-7, wherein the endonuclease is an RNA-guided endonuclease.
9. The method of any one of embodiments 1-8, wherein the RNA-guided endonuclease is a CRISPR Class 2 endonuclease.
10. The method of any one of embodiments 1-9, wherein the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.
11. The method of any one of embodiments 1-10, wherein the CRISPR Class 2 endonuclease is cas9 or cas12a.
12. The method of any one of embodiments 1-11, wherein the gene-editing nucleic acid is a guide RNA (gRNA).
13. The method of any one of embodiments 1-12, wherein the guide RNA is a single guide RNA (sgRNA).
14. The method of any one of embodiments 1-13, wherein the RNA-guided endonuclease is a CRISPR Class 1 endonuclease.
15. The method of any one of embodiments 1-14, wherein the CRISPR Class 1 endonuclease is Cas3 or Cas10.
16. The method of any one of embodiments 1-15, wherein the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).
17. The method of any one of embodiments 1-16, wherein the gene that is required for prototrophy for the nutrient is URA3, LYS2, LYS5, CAN1, amdS, FCY1, FCA1, GAP1, HSV_TK or TRP1.
18. The method any one of embodiments 1-17, wherein the protein that complements the auxotrophy for the nutrient is Kluyveromyces lactis URA3 (K1URA3).
19. The method of any one of embodiments 1-18, wherein the media that selects against expression of the protein that complements the auxotrophy for the nutrient comprises 5-FOA, alpha-aminoadipate, canavanine, fluoroacetamide, 5-fluorocytosine, D-histidine, antifolate media, or 5-fluoroanthranilic acid.
20. The method of any one of embodiments 1-19, wherein the nutrient is uracil, lysine, arginine, acetamide, cytosine, L-citrulline, FUdR or tryptophan.
21. The method of any one of embodiments 1-20, wherein the non-integrating nucleic acid construct is a plasmid.
22. A method for producing a population of gene-edited Saccharomyces cerevisiae cells free of Cas9 and sgRNA, comprising:
   (a) introducing an integrating nucleic acid construct into a population of S. cerevisiae cells that comprise a target gene of interest and that are prototrophic for uracil,
      wherein the integrating nucleic acid construct integrates into the URA3 gene; and
      wherein the integrating nucleic acid construct comprises:
         a first nucleotide sequence encoding Cas9;
         a second nucleotide sequence encoding HygR; and
         a pair of repeat nucleotide sequences flanking the first nucleotide sequence and the second nucleotide sequence;
   (b) selecting for expression of HygR to produce a population of cells that are auxotrophic for uracil;
   (c) introducing a non-integrating nucleic acid construct into the population of cells produced in step (b);
      wherein the non-integrating nucleic acid construct comprises:
         a third nucleotide sequence encoding an sgRNA that introduces an edit into the gene of interest; and
         a fourth nucleotide sequence encoding Kluyveromyces lactis URA3 (K1URA3) protein;
   (d) simultaneously selecting for expression of HygR and for prototrophy for uracil to produce a population of cells that comprise the edited gene of interest;
   (e) removing the non-integrating nucleic acid nucleic acid construct from the population of cells produced in step (d) by growing the cells on media that selects against expression of K1URA3 protein to produce a population of cells that comprise the edited gene of interest and are free of the non-integrating nucleic acid construct; and
   (f) removing the integrating nucleic acid construct from the population of cells produced in step (e) by growing the cells on media that selects for prototrophy for uracil to produce a population of cells that comprise the edited gene of interest and that are free of the integrating nucleic acid construct.
23. A population of cells comprising a nucleic acid construct integrated into a gene that is required for prototrophy for a nutrient, wherein the integrated nucleic acid construct comprises:
   a first nucleotide sequence encoding a gene-editing protein;
   a second nucleotide sequence encoding a dominant selectable marker; and
   a pair of repeat nucleotide sequences flanking the first nucleotide sequence and the second nucleotide sequence.
24. The population of embodiment 23, further comprising a non-integrating nucleic acid construct, wherein the non-integrating nucleic acid construct comprises:
   a third nucleotide sequence encoding a gene-editing nucleic acid that introduces an edit into a gene of interest; and
   a fourth nucleotide sequence encoding a protein that complements the auxotrophy for the nutrient, wherein the fourth nucleotide sequence cannot recombine with the cellular genome.
25. A population of cells comprising an edited gene of interest and a nucleic acid construct integrated into a gene that is required for prototrophy for a nutrient, wherein the integrated nucleic acid construct comprises:
   a first nucleotide sequence encoding a gene-editing protein;
   a second nucleotide sequence encoding a dominant selectable marker; and
   a pair of repeat nucleotide sequences flanking the first nucleotide sequence and the second nucleotide sequence.
26. The population of any one of embodiments 23-25, wherein the cells are fungal cells or bacterial cells.
27. The population of any one of embodiments 23-26, wherein the fungal cells are Fusarium spp., Kluyveromyces spp., Penicillium spp., Pichia spp., Saccharomyces spp., Schizosaccharomyces spp. or Yarrowia spp.
28. The population of any one of embodiments 23-27, wherein the fungal cells are Kluyveromyces lactis, Kluyveromyces marxianus, Pichia pastoris, Saccharomyces cerevisiae, Schizosaccharomyces pombe or Yarrowia lipolytica.
29. The population of any one of embodiments 23-28, wherein the bacterial cells are Agrobacterium spp., Arthrobacterspecies spp., Bacillus spp., Clostridium spp., Corynebacterium spp., Cupriavidus spp., Escherichia spp., Erwinia spp., Geobacillus spp., Lactobacillus spp., Pantoea spp., Propionibacterium spp., Pseudomonas spp., Sphingomonas spp., Streptococcus spp., Streptomyces spp., Xanthomonas spp., or Zymomonas spp.
30. The population of any one of embodiments 23-29, wherein the bacterial cells are Bacillus clausii, Bacillus licheniformis, Bacillus subtilis, Clostridium acetobutylicum, Corynebacterium glutamicum, Cupriavidus necator, Escherichia coli, Geobacillus thermoglucosidasius, Propionibacterium freudenreichii, Sphingomonas elodea, or Xanthomonas campestris.
31. The population of any one of embodiments 23-30, wherein the gene-editing protein is an endonuclease.
32. The population of any one of embodiments 23-31, wherein the endonuclease is an RNA-guided endonuclease.
33. The population of any one of embodiments 23-32, wherein the RNA-guided endonuclease is a CRISPR Class 2 endonuclease.
34. The population of any one of embodiments 23-33, wherein the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.
35. The population of any one of embodiments 23-34, wherein the CRISPR Class 2 endonuclease is cas9 or cas12a.
36. The population of any one of embodiments 23-35, wherein the gene-editing nucleic acid is a guide RNA (gRNA).
37. The population of any one of embodiments 23-36, wherein the guide RNA is a single guide RNA (sgRNA).
38. The population of any one of embodiments 23-37, wherein the RNA-guided endonuclease is a CRISPR Class 1 endonuclease.
39. The population of any one of embodiments 23-38, wherein the CRISPR Class 1 endonuclease is Cas3 or Cas10.
40. The population of any one of embodiments 23-39, wherein the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).
41. The population of any one of embodiments 23-40, wherein the gene that is required for prototrophy for the nutrient is URA3, LYS2, LYS5, CAN1, amdS, FCY1, FCA1, GAP1, HSV_TK or TRP1.
42. The population any one of embodiments 23-41, wherein the protein that complements the auxotrophy for the nutrient is Kluyveromyces lactis URA3 (K1URA3).
43. The population of any one of embodiments 23-42, wherein the nutrient is uracil, lysine, arginine, acetamide, cytosine, L-citrulline, FUdR or tryptophan.
44. The population of any one of embodiments 23-43, wherein the non-integrating nucleic acid construct is a plasmid.
45. A method for producing a population of multiply gene-edited cells free of gene-editing system molecules, comprising:
   (a) introducing a first integrating nucleic acid construct into a first population of cells that comprise a first edited gene of interest and that are prototrophic for a nutrient,
      wherein the first integrating nucleic acid construct integrates into a gene that is required for prototrophy for the nutrient; and
      wherein the first integrating nucleic acid construct comprises:
         a first nucleotide sequence encoding a protein that enables mating;
         a second nucleotide sequence encoding a first dominant selectable marker; and
         a pair of repeat nucleotide sequences flanking the first nucleotide sequence and the second nucleotide sequence;
   (b) introducing a second integrating nucleic acid construct into a second population of cells that comprise a second edited gene of interest and that are prototrophic for a nutrient,
      wherein the second integrating nucleic acid construct integrates into a gene that is required for prototrophy for the nutrient; and
      wherein the second integrating nucleic acid construct comprises:
         a third nucleotide sequence encoding a protein that enables mating;
         a fourth nucleotide sequence encoding a second dominant selectable marker; and
         a pair of repeat nucleotide sequences flanking the third nucleotide sequence and the fourth nucleotide sequence;
   (c) selecting for expression of the first dominant selectable marker within the first population of cells and selecting for expression of the second dominant selectable marker within the second population of cells to produce first and second populations of cells that are auxotrophic for the nutrient and mating-competent;
   (d) sporulating the first and second population of cells of step (c) to produce first and second populations of meiotic progeny;
   (e) allowing the first and second populations of meiotic progeny to mate with each other, thereby producing a mated population of cells;
   (f) simultaneously selecting for expression of the first and second dominant selectable markers within the mated population of cells to produce cells comprising genetic information from both the first and second populations of cells;
   (g) sporulating the mated population of cells of step (f) to allow recombination of the first and second edited genes of interest into a single genome; and
   (h) removing the integrating nucleic acid construct from the population of cells produced in step (g) by growing the cells on media that selects for prototrophy for the nutrient to produce a population of cells that comprise the edited genes of interest and that are free of the integrating nucleic acid constructs.
46. The method of embodiment 45, wherein the cells are fungal cells.
47. The method of any one of embodiments 45-46, wherein the fungal cells are Fusarium spp., Kluyveromyces spp., Penicillium spp., Pichia spp., Saccharomyces spp., Schizosaccharomyces spp. or Yarrowia spp.
48. The method of any one of embodiments 45-47, wherein the fungal cells are Kluyveromyces lactis, Kluyveromyces marxianus, Pichia pastoris, Saccharomyces cerevisiae, Schizosaccharomyces pombe or Yarrowia lipolytica.
49. The method of any one of embodiments 45-48, wherein the protein that enables mating is one that enables mating-type switching.
50. The method of any one of embodiments 45-49, wherein the protein is the HO endonuclease.
51. The method of any one of embodiments 45-50, wherein the first or second dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).
52. The method of any one of embodiments 45-51, wherein the gene that is required for prototrophy for the nutrient is URA3, LYS2, LYS5, CAN1, amdS, FCY1, FCA1, GAP1, HSV_TK, or TRP1.
53. The method of any one of embodiments 45-52, wherein the nutrient is uracil, lysine, arginine, acetamide, cytosine, L-citrulline, FUdR, or tryptophan.
54. A method for producing a population of multiply gene-edited yeast cells free of HO nuclease and antibiotic resistance markers, comprising:
   (a) introducing a first integrating nucleic acid construct into a first population of haploid yeast cells that comprise a first edited gene of interest and that are prototrophic for tryptophan,
      wherein the first integrating nucleic acid construct integrates into the TRP1 gene; and
      wherein the first integrating nucleic acid construct comprises:
         a first nucleotide sequence encoding HO nuclease;
         a second nucleotide sequence encoding a kanamycin or hygromycin antibiotic resistance gene; and
         a pair of repeat nucleotide sequences flanking the first nucleotide sequence and the second nucleotide sequence;
   (b) introducing a second integrating nucleic acid construct into a second population of haploid yeast cells that comprise a second edited gene of interest and that are prototrophic for tryptophan,
      wherein the second integrating nucleic acid construct integrates into the TRP1 gene; and
      wherein the second integrating nucleic acid construct comprises:
         a third nucleotide sequence encoding HO nuclease;
         a fourth nucleotide sequence encoding the other of a kanamycin or hygromycin antibiotic resistance gene not encoded by the second nucleotide sequence; and
         a pair of repeat nucleotide sequences flanking the third nucleotide sequence and the fourth nucleotide sequence;
   (c) selecting for expression of the antibiotic resistance gene encoded by the second nucleotide sequence within the first population of yeast cells and selecting for expression of the antibiotic resistance gene encoded by the fourth nucleotide sequence within the second population of yeast cells to produce first and second populations of cells that are auxotrophic for tryptophan and mating-competent;
   (d) sporulating the first and second population of yeast cells of step (c) to produce first and second populations of meiotic progeny;
   (e) allowing the first and second populations of auxotrophic, mating-competent yeast cells to mate with each other, thereby producing a mated population of cells;
   (f) simultaneously selecting for expression of both antibiotic resistance genes within the mated population of yeast cells to produce yeast cells comprising genetic information from both the first and second populations of yeast cells;
   (g) sporulating the mated population of yeast cells of step (f) to allow recombination of the first and second edited genes of interest into a single genome; and
   (h) removing the integrating nucleic acid construct from the population of yeast cells produced in step (e) by growing the yeast cells on media that selects for tryptophan prototrophy to produce a population of yeast cells that comprise the edited genes of interest and that are free of the integrating nucleic acid constructs.
55. A population of cells comprising a nucleic acid construct integrated into a gene that is required for prototrophy for a nutrient, wherein the integrated nucleic acid construct comprises:
   a first nucleotide sequence encoding a protein that enables mating;
   a second nucleotide sequence encoding a dominant selectable marker; and
   a pair of repeat nucleotide sequences flanking the first nucleotide sequence and the second nucleotide sequence.
56. A population of cells comprising multiple edited genes of interest and two nucleic acid constructs integrated into a gene that is required for prototrophy for a nutrient, wherein the first integrated nucleic acid construct comprises:
   a first nucleotide sequence encoding a protein that enables mating;
   a second nucleotide sequence encoding a dominant selectable marker; and
   a pair of repeat nucleotide sequences flanking the first nucleotide sequence and the second nucleotide sequence; and
wherein the second integrated nucleic acid construct comprises:
   a third nucleotide sequence encoding a protein that enables mating;
   a fourth nucleotide sequence encoding a second dominant selectable marker; and
   a pair of repeat nucleotide sequences flanking the third nucleotide sequence and the fourth nucleotide sequence.
57. The population of any one of embodiments 55-56, wherein the cells are fungal cells.
58. The population of any one of embodiments 55-57, wherein the fungal cells are Fusarium spp., Kluyveromyces spp., Penicillium spp., Pichia spp., Saccharomyces spp., Schizosaccharomyces spp. or Yarrowia spp.
59. The population of any one of embodiments 55-58, wherein the fungal cells are Kluyveromyces lactis, Kluyveromyces marxianus, Pichia pastoris, Saccharomyces cerevisiae, Schizosaccharomyces pombe or Yarrowia ipolytica.
60. The population of any one of embodiments 55-59, wherein the protein that enables mating is one that enables mating-type switching.
61. The population of any one of embodiments 55-60, wherein the protein is the HO endonuclease.
62. The population of any one of embodiments 55-61, wherein the first or second dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).
63. The population of any one of embodiments 55-62, wherein the gene that is required for prototrophy for the nutrient is URA3, LYS2, LYS5, CAN1, amdS, FCY1, FCA1, GAP1, HSV_TK, or TRP1.
64. The population of any one of embodiments 55-63, wherein the nutrient is uracil, lysine, arginine, acetamide, cytosine, L-citrulline, FUdR, or tryptophan.
65. A Removal by Prototrophic Selection (RePS) polynucleotide for genetic engineering via integration into a gene that is required for prototrophy for a nutrient, the polynucleotide comprising
   (a) a first nucleotide sequence encoding a gene-editing protein or a protein that enables mating;
   (b) a second nucleotide sequence encoding a dominant selectable marker; and
   (c) a pair of repeat nucleotide sequences flanking the first nucleotide sequence and the second nucleotide sequence,
   wherein the repeats of (c) allow for recombination to restore the gene that is required for prototrophy for the nutrient while removing the first and second nucleotide sequences.
66. The polynucleotide of embodiment 65, wherein the gene-editing protein is an endonuclease.
67. The polynucleotide of any one of embodiments 65-66, wherein the endonuclease is an RNA-guided endonuclease.
68. The polynucleotide of any one of embodiments 65-67, wherein the RNA-guided endonuclease is a CRISPR Class 2 endonuclease.
69. The polynucleotide of any one of embodiments 65-68, wherein the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.
70. The polynucleotide of any one of embodiments 65-69, wherein the CRISPR Class 2 endonuclease is cas9 or cas12a.
71. The polynucleotide of any one of embodiments 65-70, wherein the RNA-guided endonuclease is a CRISPR Class 1 endonuclease.
72. The polynucleotide of any one of embodiments 65-71, wherein the CRISPR Class 1 endonuclease is Cas3 or Cas10.
73. The polynucleotide of any one of embodiments 65-72, wherein the protein that enables mating is one that enables mating-type switching.
74. The polynucleotide of any one of embodiments 65-73, wherein the protein is the HO endonuclease.
75. The polynucleotide of any one of embodiments 65-74, wherein the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).
76. The polynucleotide of any one of embodiments 65-75, wherein the gene that is required for prototrophy for the nutrient is URA3, LYS2, LYS5, CAN1, amdS, FCY1, FCA1, GAP1, HSV_TK, or TRP1.
77. The polynucleotide of any one of embodiments 65-76, wherein the nutrient is uracil, lysine, arginine, acetamide, cytosine, L-citrulline, FUdR, or tryptophan.

1. A method for producing a population of gene-edited cells free of gene-editing system molecules, comprising:
(a) introducing an integrating nucleic acid construct into a population of cells that comprise a target gene of interest and that are prototrophic for a nutrient,
wherein the integrating nucleic acid construct integrates into a gene that is required for prototrophy for the nutrient; and
wherein the integrating nucleic acid construct comprises:
a first nucleotide sequence encoding a gene-editing protein;
a second nucleotide sequence encoding a dominant selectable marker; and
a pair of repeat nucleotide sequences flanking the first nucleotide sequence and the second nucleotide sequence;
(b) selecting for expression of the dominant selectable marker to produce a population of cells that are auxotrophic for the nutrient;
(c) introducing a non-integrating nucleic acid construct into the population of cells produced in step (b);
wherein the non-integrating nucleic acid construct comprises:
a third nucleotide sequence encoding a gene-editing nucleic acid that introduces an edit into the gene of interest; and
a fourth nucleotide sequence encoding a protein that complements the auxotrophy for the nutrient, wherein the fourth nucleotide sequence cannot recombine with the cellular genome;
(d) simultaneously selecting for expression of the dominant selectable marker and for prototrophy for the nutrient to produce a population of cells that comprise the edited gene of interest;
(e) removing the non-integrating nucleic acid nucleic acid construct from the population of cells produced in step (d) by growing the cells on media that selects against expression of the protein that complements the auxotrophs for the nutrient to produce a population of cells that comprise the edited gene of interest and are free of the non-integrating nucleic acid construct; and
(f) removing the integrating nucleic acid construct from the population of cells produced in step (e) by growing the cells on media that selects for prototrophy for the nutrient to produce a population of cells that comprise the edited gene of interest and that are free of the integrating nucleic acid construct.

2. The method of claim 1, wherein the cells are fungal cells or bacterial cells.

3. The method of claim 2, wherein the fungal cells are Fusarium spp., Kluyveromyces spp., Penicillium spp., Pichia spp., Saccharomyces spp., Schizosaccharomyces spp. or Yarrowia spp.

4. The method of claim 2, wherein the fungal cells are Kluyveromyces Kluyveromyces marxianus, Pichia pastoris, Saccharomyces cerevisiae, Schizosaccharomyces pombe or Yarrowia lipolytica.

5. The method of claim 2, wherein the bacterial cells are Agrobacterium spp., Arthrobacterspecies spp., Bacillus spp., Clostridium spp., Corynebacterium spp., Cupriavidus spp., Escherichia spp., Erwinia spp., Geobacillus spp., Lactobacillus spp., Pantoea spp., Propionibacterium spp., Pseudomonas spp., Sphingomonas spp., Streptococcus spp., Streptomyces spp., Xanthomonas spp., or Zymomonas spp.

6. The method of claim 2, wherein the bacterial cells are Bacillus clausii, Bacillus licheniformis, Bacillus subtilis, Clostridium acetobutylicum, Corynebacterium Cupriavidus necator, Escherichia coli, Geobacillus thermoglucosidasius, Propionibacterium freudenreichii, Sphingomonas elodea, or Xanthomonas campestris.

7. The method of claim 1, wherein the gene-editing protein is an endonuclease.

8. The method of claim 7, wherein the endonuclease is an RNA-guided endonuclease.

9. The method of claim 8, wherein the RNA-guided endonuclease is a CRISPR Class 2 endonuclease.

10. The method of claim 9, wherein the CRISPR Class 2 endonuclease is selected from the list consisting of: cas9, cas12a, cas12b1, cas12b2, cas12c, cas12d, cas12e, cas12f1, cas12f2, cas12f3, cas12g, cas12h, cas12i, cas12k, cas13a, cas13b1, cas13b2, cas13c, cas13d, c2c4, c2c8, c2c9, c2c10, and Cms1 endonucleases.

11. The method of claim 9, wherein the CRISPR Class 2 endonuclease is cas9 or cas12a.

12. The method of claim 1, wherein the gene-editing nucleic acid is a guide RNA (gRNA).

13. The method of claim 12, wherein the guide RNA is a single guide RNA (sgRNA).

14. The method of claim 8, wherein the RNA-guided endonuclease is a CRISPR Class 1 endonuclease.

15. The method of claim 14, wherein the CRISPR Class 1 endonuclease is Cas3 or Cas10.

16. The method of claim 1, wherein the dominant selectable marker is hygromycin B phosphotransferase (hygR), nourseothricin N-acetyl transferase (Nat), KanMX, patMX, zeocin antibiotic resistance (Zeo), AmdS, or thymidine kinase (Tk).

17. The method of claim 1, wherein the gene that is required for prototrophy for the nutrient is URA3, LYS2, LYS5, CAN1, amdS, FCY1, FCA1, GAP1, HSV_TK or TRP1.

18. The method of claim 17, wherein the protein that complements the auxotrophy for the nutrient is Kluyveromyces lactis URA3 (KIURA3).

19. The method of claim 18, wherein the media that selects against expression of the protein that complements the auxotrophy for the nutrient comprises 5-FOA, alpha-aminoadipate, canavanine, fluoroacetamide, 5-fluorocytosine, D-histidine, antifolate media, or 5-fluoroanthranilic acid.

20. The method of claim 1, wherein the nutrient is uracil, lysine, arginine, acetamide, cytosine, L-citrulline, FUdR or tryptophan.

21-77. (canceled)
